(19) United States
(12) Patent Application Publication
(10) Pub. No.: US 2021/0069268 A1
(43) Pub. Date: Mar. 11, 2021

(54) METABOLOMIC REVISION OF MAMMALIAN INFANTS

(71) Applicant: EVOLVE BIOSYSTEMS, INC., Davis, CA (US)

(72) Inventors: Bethany HENRICK, Davis, CA (US); Steven FRESE, Davis, CA (US); David KYLE, Davis, CA (US); Samara FREEMAN-SHARKEY, Davis, CA (US); Giorgio CASABURI, Davis, CA (US)

(73) Assignee: Evolve Biosystems, Inc., Davis, CA (US)

(21) Appl. No.: 16/646,687

(22) PCT Filed: Sep. 13, 2018
(86) PCT No.: PCT/US2018/050973

§ 371 Date: Mar. 12, 2020

Related U.S. Application Data
(60) Provisional application No. 62/558,349, filed on Sep. 13, 2017.

(51) Int. Cl. A61K 35/747; A61K 31/198; A61K 35/20; A61K 35/745; A61K 35/744; A61P 1/14

(52) U.S. Cl. CPC A61K 35/747; A61K 31/198; A61P 1/14; A61K 35/745; A61K 35/744; A61K 35/20

(57) ABSTRACT

The inventions described herein relate generally to the use of compositions to increase output of particular metabolites in the gut of a nursing infant mammal including humans. These compositions generally comprise one or more bacterial strains selected for their growth on mammalian milk oligosaccharides, a source of mammalian milk oligosaccharides, and, optionally, nutritive components required for the growth of that infant mammal.

### FIELD OF INVENTION

The inventions described herein relate generally to the use of compositions and methods designed to provide a targeted, renewable source of key metabolites and/or their precursors to the intestine of a mammal where that mammal may be an infant, or a non-infant with a specific metabolic condition, or an individual who is in need of intestinal maturation or restoration. These include, but are not limited to gut programming. The inventions also relate to methods of providing a functional readout on the status of such metabolites and functional interactions between the gut microbiome and its host. The compositions of these inventions generally comprise oligosaccharides of the sort found in mammalian milk and/or one or more bacterial strains selected for their ability to outcompete other intestinal bacteria when grown on oligosaccharides found in mammalian milk.

### BACKGROUND

Metabolites are the intermediates and products of the life-sustaining chemical transformations which occur within the cells of living organisms. Metabolomics is the systematic study of the unique chemical fingerprints that specific cellular processes leave behind (i.e., the study of their small-molecule metabolite profiles). The metabolome represents the collection of all metabolites in a biological cell, tissue, organ, or organism, which are the end products of cellular processes. In the case of fecal samples, this includes a combination of host metabolites and bacterial metabolites.

Human and microbial metabolites may or may not have identified functions within defined pathways that may make them useful or harmful to the host. Metabolomic analyses are useful in providing unanticipated insights into health and disease states of the host. Metabolomic profiles have been used to predict the progression of disease. For example, plasma biomarkers have been used to compare metabolic profiles of individuals at risk for insulin resistance and insulin resistance related disorders such as Type 2 diabetes to predict the progress to disease three to five years in the future (Gall et al., US Pub. No. 2015/0362510). In this case, hundreds of metabolites were reported as being different, including creatine, gamma-glutamyltyrosine, gamma-glutamylphenylalanine, gamma-glutamylglutamine, 3-hydroxyhippurate, 4-hydroxyhippurate, hippurate, phenyllactate, serotonin, leucylleucine, glycerophosphorylcholine which were modestly changed between progressors and non-progressors with some being increased and some being decreased and associated via statistical method to disease risk 3 to 5 years later.

### SUMMARY OF INVENTION

Creating a healthy intestinal environment is important for the overall health of the mammal. The inventors have discovered a means of providing or removing key metabolites and/or their precursors in the intestine in amounts sufficient to change the overall intestinal metabolome. The abundance of key metabolites can act in nutritive, absorptive, metabolic and immunological functions to promote the overall health of the mammal. These metabolites can also be administered in a therapeutic capacity to restore homoeostasis in conditions of altered metabolic (i.e., obesity, Type 2 diabetes) and cognitive function (i.e., cognitive development, learning, depression).

These metabolites may be increased or decreased alone or in combination to modulate the physiology and biochemistry of the infant gut. The present invention provides for compositions, methods and protocols to provide adequate levels of these compounds to restore and promote nutritional and metabolic health of the intestine, and health of other key organs including the liver and central nervous system. Monitoring the status of the some or all of the metabolites may be used to identify persons at risk of developing diseases in the future. Individuals identified as being at risk for a particular disease or condition may be clinically monitored at a future date to demonstrate the absence or reduction in symptoms associated with said disease or condition.

Compositions and methods described in this application provide the means of altering the metabolome to prevent gut dysfunction. Unhealthy levels of certain metabolites (insufficient or excess metabolites) in the gut can also be described as fecal dysmetabolosis or a dysmetabolic state. The inventors discovered that in developed countries, such as the US, presumed healthy newborn infants are unexpectedly deficient in important gut metabolites and/or their precursors that are important for reducing oxidative stress, for metabolic regulation of food intake (satiety), for brain growth and development including cognition, and as facilitators of detoxification of certain polyphenols like benzoic acid, among other things. These sub-clinical findings may be important predictors of long-term health and chronic deficiencies and may lead to an increased risk for inappropriate gut development or maturation leading to conditions such as, but not limited to, metabolic disorders, including Type 2 diabetes and/or obesity, and/or Type 1 diabetes, allergy, atopy, asthma.

In particular, this invention provides methods of monitoring the health of mammals. The health of the mammals can be monitored by (a) obtaining a fecal sample from the mammal; (b) determining the amount of metabolites in the sample; and/or (c) identifying a healthy state versus a dysmetabolic state in the mammal based on the abundance or deficiency of the metabolites in the sample. The metabolites can be some of the metabolites listed in column 2 of Table 1. In some embodiments, the method of monitoring the health of a mammal can include treating a dysmetabolic mammal by administering bacteria, mammalian milk oligosaccharides (MMO) or both. In some embodiments, a method of monitoring health can include treating a dysmetabolic mammal by administering bacteria, selective oligosaccharides (OS) or both. Selective oligosaccharides are typically DP3 to DP20, selective oligosaccharides are more preferably DP3 to DP10 and may be from any source: chemical synthesis; plant; algae; yeast; bacteria; or mammals. The oligosaccharides administered are typically functional equivalents to Mammalian milk oligosaccharides (MMO). In some embodiments OS and MMO are structurally equivalent. The bacteria typically comprise bacteria capable of colonization in the mammalian colon. The bacteria and/or the OS can be administered in respective amounts to change the abundance of the one or more metabolites in the feces of the mammal to a non-dysmetabolic level.

The invention also provides methods of maintaining the health of a mammal by administering bacteria and/or OS. Additionally, a fecal sample can be obtained from the mammal, and the level of metabolite(s) in the sample can be determined. A metabolic state in the mammal can be identified based on the concentration and/or level and/or content of the metabolite(s) in the sample, and the bacteria and/or the OS can be administered in response to the identified dysmetabolic state.

In some embodiments, the health of a mammal can be maintained by administering bacteria or OS in an amount sufficient to change the level of metabolite(s). The amount, periodicity, and/or duration of the bacteria and/or OS that is administered can be different than the amount of the bacteria and/or OS administered in response to an identified dysmetabolic state. The bacteria can be capable of colonization of the colon.

In some embodiments, the invention provides a method of decreasing and/or maintaining a low level of metabolite(s) in the colon of a mammal by (a) administering a bacteria; and (b) administering OS; where the bacteria and the OS are administered in respective amounts sufficient to maintain a level of one or more metabolites in the feces of said mammal. In some embodiments, this method includes one or more metabolites selected from the compounds listed in column 2 of Table 1. In another embodiment, the levels of the bacteria and/or the metabolite can be modulated by altering the level of OS in the diet

The invention also provides methods of establishing or altering an infant, non-infant or specific mammal's gut metabolome by administering particular bacteria and/or OS and monitoring the mammal. The mammal can be monitored by obtaining a fecal or systemic sample and determining the increase or decrease in metabolites, such as but not limited to, serotonin metabolites, tryptophan metabolites, and/or lactate conjugates such as 3-4 hydroxyphenyl lactate, indole lactate and/or phenyllactate. Evaluating the alteration of metabolite relative to a healthy state will allow for the administration of bacteria and/or OS in response to the identified dysmetabolic state.

In any of the above embodiments, the metabolite(s) concentrations that are monitored or altered include those such as, but not limited to γ-glutamyl-containing di- or tri-peptides, pipecolic acid, hippurate, serotonin, tryptophan, and/or lactate conjugates such as 3-4 hydroxyphenyl lactate, indole lactate and/or phenyllactate. In some embodiments, the abundance of γ-glutamyl-cysteine is at least 20-fold greater than the γ-glutamyl-cysteine abundance in the colon of a human infant, which is not colonized by the administered bacteria. In a preferred embodiment, the level of pipecolic acid or salt thereof is at least 10-fold greater than the pipecolic acid level in the colon of a human infant, which is not colonized by the bacteria. In a preferred embodiment, the level of γ-glutamyl-containing di- or tri-peptides is at least 10-fold greater than the level in the colon of a human infant which is not colonized by the bacteria. Using the above embodiments, oxidative stress of an infant can be reduced. Additionally, bacterial hippurate degraders can be reduced. Jaundice can be prevented or ameliorated.

In another embodiment, one or more metabolites comprises pipecolic acid or a salt thereof. In another embodiment, the level of pipecolic acid or salt thereof is at least 10-fold greater than the pipecolic acid level in the colon of a human infant which is not colonized by said bacteria.

Any of the methods described herein can reduce the risk of a mammal developing metabolic disorders such as, but not limited to Juvenile Diabetes (Type I), obesity, asthma, atopy, Celiac's Disease, food allergies, autism, as compared to a dysmetabolic mammal. The risk can be reduced by 20%, 30%, 40%, 50%, 60%, 70%, 80%, or 90%.

In any of the above embodiments, the oligosaccharides can include the carbohydrate polymers found in mammalian milk, which are not metabolized by any combination of digestive enzymes expressed by mammalian genes. The selective oligosaccharides composition can include one or more of lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose (3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof. The OS can include: (a) a Type II oligosaccharide core where representative species include LnNT; (b) one or more oligosaccharides containing the Type II core and GOS in 1:5 to 5:1; (c) one or more oligosaccharides containing the Type II core and FL 1:5 to 5:1; or (d) one or more oligosaccharides containing the Type II core and SL 1:5 to 5:1; or (e) a combination of (a)-(d). The OS can include: (a) a Type I oligosaccharide core where representative species include LNT; (b) one or more oligosaccharides containing the Type I core and GOS in 1:5 to 5:1; (c) one or more oligosaccharides containing the Type I core and FL 1:5 to 5:1; (d) one or more oligosaccharides containing the Type I core and SL 1:5 to 5:1; or (e) a combination of (a)-(d). In some embodiments, Type I and Type II oligosaccharides in combination with any of GOS, FL, or SL. Type I or type II may be isomers of each other. Other type II cores include but are not limited to trifucosyllacto-N-hexaose (TFLNH), LnNH, lacto-N-hexaose (LNH), lacto-N-fucopentaose III (LNFPIII), monofucosylated lacto-N-Hexose III (MFLNHIII), Monofucosylmonosialyllacto-N-hexose (MFMSLNH).

The mammal can receive OS at a dose of over 25%, 40%, or 50% of the mammal's total dietary fiber. Over 10%, 25%, 40%, 50%, 60%, or 75% of the total oligosaccharide can be represented by one or more units of N-acetyllactosamine (Type II core). Additionally, the oligosaccharide composition can include 2′FL and/or GOS.

The OS can be administered prior to, contemporaneously with, within 2 hours or, or after the administration of the bacteria. The OS and/or bacteria can be administered for at least 1, 3, 10, at least 20, at least 30, at least 60, at least 90, at least 120, at least 150, or at least 180 days.

In any of the above embodiments, the bacteria can be capable of colonization in the colon. For example, the bacteria can be from the genus of Bifidobacteria, Lactobacillus, or Pediococcus, such as B. adolescentis, B. animalis, B. animalis subsp. animalis, B. animalis subsp. lactis, B. bifidum, B. breve, B. catenulatum, B. longum, B. longum subsp. infantis, B. longum subsp. longum, B. pseudocatanulatum, B. pseudolongum, L. acidophilus, L. antri, L. brevis, L. casei, L. coleohominis, L. crispatus, L. curvatus, L. fermentum, L. gasseri, L. johnsonii, L. mucosae, L. pentosus, L. plantarum, L. reuteri, L. rhamnosus, L. sakei, L. sahvarius, P. acidilactici, P. argentinicus, P. claussenii, P. pentosaceus, P. stilesii L. paracasei, L. kisonensis., L. paralimentarius, L. perolens, L. apis, L. ghanensis, L. dextrinicus, L. shenzenensis, L. harbinensis, P. parvulus, or P. lolii.

In any of the above embodiments, the bacteria can be Bifidobacterium longum subsp. infantis EVC001 as deposited under ATCC Accession No PTA-125180; cells were deposited with the American Type Culture Collection at 10801 University Blvd, Manassas, Va. 20110 under the Budapest Treaty on the International Recognition of the Deposit of Microorganisms for the Purposes of Patent Procedure, the “Deposited Bacteria.”

Additionally, “Deposited Bacteria,” as used herein, refers to the isolated Bifidobacterium longum subsp. infantis EVC001, deposited with the ATCC and assigned Accession Number, and variants thereof, wherein said variants retain the phenotypic and genotypic characteristics of said bacteria and wherein said bacteria and variants thereof have LNT transport capability and comprise a functional H5 gene cluster comprising BLON2175, BLON2176, and BLON2177.

A “functional H5 cluster,” refers to a cluster of genes in Bifidobacteria responsible for the uptake and metabolism of human milk oligosaccharides. A functional H5 cluster comprises Blon_2175, Blon_2176, and Blon_2177. The H5 cluster comprises the following genes: Blon_2171, Blon_2173, Blon_2174, Blon_2175, Blon_2176, Blon_2177, and galT.

In any of the above embodiments, the mammal can be a human, such as an infant, an adolescent, an adult, or a geriatric adult. The mammal can be an infant human that is dysbiotic.

### BRIEF DESCRIPTION OF THE FIGURES

FIG. 1. Amount (CFU/g) of B. longum subsp. infantis (B. infantis) in fecal samples as measured by qPCR during the intervention period and a follow-up period in both vaginally- and C-section-delivered human infants. The black line and dots represent all infants who were supplemented with B. infantis for 21 days starting at 7 days of life. All infants receiving the standard of care (no probiotic) are depicted with the grey line and dots. The band around each line represents a 95% confidence interval around the line. The end of supplementation occurred at day 28 and samples were collected until day 60 of life.

FIG. 2A. Abundances of different genera of intestinal bacteria in an untreated C-section baby over the study period (Day 6 to 60 of life).

FIG. 2B. Abundance of different genera of intestinal bacteria in a C-section baby treated from Day 7 to 28 with B. longum subsp. infantis.

FIG. 3A. Principal component analysis of all samples. Each dot represents one sample.

FIG. 3B. Statistical summary of the analysis of all samples.

### DETAILED DESCRIPTION OF THE INVENTION

This invention is directed to methods of monitoring, treating and/or preventing metabolic dysfunction, metabolite insufficiency or metabolite excesses in infant mammalian intestines (“dysmetabolosis”), and to compositions and methods that generate and/or deliver certain metabolites and/or their precursors to the intestine whereby altered levels of these metabolites may be found systemically.

The inventors have discovered that there are significant decreases in certain metabolites or their precursors in stool samples from the infant population in the US, and limitation of these metabolites may significantly affect the immediate or long-term health of those infants and may be monitored. This includes compounds such as, but not limited to, phenyllactate, 3-(4-hydroxyphenyl)lactate, indolelactate, indole-3-lactic acid, isovalerylcarnitine (C5), N-acetylcysteine, taurine, citrate, arginine, creatinine, 5-oxoproline, gamma-glutamylcysteine, gamma-glutamylhistidine, gamma-glutamylmethionine, pyruvate, lactate, including fatty acid hydroxyl fatty acids such as palmitic acid-9-hydroxy-stearic acid (PAHSA 16:0/OH-18:0), and Oleic Acid-Hydroxy Stearic Acid (OAHSA 18:1/OH-18:0), nonadeconate, arachinate, eiocosenoate, stearate, 15-methylplamitate, 17-methylpalmitate, behenate, margarate, palmitate, myristate. 8-hydroxyguanine, 2′-O-methylcytidine, thiamin (Vitamin B1), N-acetylglucosamine 6-sulfate, glutamate, propionylglutamine, N6-formyllysine, N6-acetyllysine, N-acetylproline, alanylleucine, lysyl leucine, phenylacetylglutamate, glycerol, gluconate, arachidate, and/or sphingomyelin. These compounds may be increased with compositions used in this invention.

Conversely, the inventors have also discovered that there are significant increases in metabolites in stool samples which may have undesirable consequences for the infant population in the US and may be monitored. These compounds include, but are not limited to fecal betaine, benzoic acid, N-acetylglycine, cadaverine, tyramine, agmatine, putrescene, spermidine, imidazole proprionate, leucylglycine, phenylalanylglycine, 2-hydroxymethylvalerate, alpha-hydroxyisovalerate, alpha-hydroxyisocaproate, taurocholate, lysylphospolipids, succinate, fumarate, taurolithocholate 3-sulfate, indoleacetate, 4-hydroxyphenylpyruvate, 4-hydroxybenzoate, cystine, 2-methylmalonylcarnithine, and/or glycylisoleucine. These compounds are decreased with compositions used in this invention.

Alterations include but are not limited to the TCA cycle (energy status), protein digestion, lipid degradation, carbohydrate utilization, neurotransmitter availability, glutathione metabolism, redox systems, vitamin production, amino acid metabolism (i.e lysine, tryptophan, phenylalanine, tyrosine, glutamate, cysteine, proline), primary and secondary bile acid metabolism, bile acid malabsorption conditions, nucleic acid metabolism including adenosine metabolism, sphingolipid metabolism, tocopherol metabolism, tetrahydrobiopterin metabolism, and xanthine metabolism, blood clotting mechanisms.

Metabolites may come exclusively from the bacteria, such as hippurate, 2-hydroxyhippurate, 3-hydroxyhippurate, 4-hydroxyhippurate, 4-hydroxybenzoate, indolelactate, indoleacetate, cadaverine, phenylacetate, phenyllactate, 3-(4-hydroxyphenyllactate), and 4-hydroxyphenylpyruvate. N-acetylglucosamine 6-sulfate is a key metabolite in the Bif-shunt. Other metabolites may be contributed by host and/or microbes.

Metabolites such as citrate and succinate (Table 1 TCA cycle metabolites) can be used alone or together to assess at least one functional output, such as energy status and tissue repair. Methods include increasing citrate and/or lowering succinate levels in the intestine. A ratio of citrate/succinate can be used to monitor and reduce risk of obesity, diabetes and other metabolic disorders.

Metabolites from Table 1 may be selected to for their ability to behave as anti-oxidants to reduce reactive oxygen species. Examples from Table 1 include cysteine and choline.

Methods to increase at least choline, and/or primary bile salts such as cholate and chenodeoxycholate and/or lower secondary bile acids may be used in clinical situations to reduce reactive oxygen species, improve liver function and/or reduce risk liver disease.

This invention provides compositions and methods of use to provide and/or remove metabolites and/or their precursors to support intestinal, liver and central nervous system health. Generally, the key components are delivered through administering a food composition comprising selective oligosaccharides (OS) that are mammalian milk oligosaccharides (MA/10) or functional equivalents thereof to a mammal in conjunction with a bacterial composition comprising bacteria capable of increasing or decreasing availability of certain metabolites and/or their precursors. The OS including MMO and their functional equivalents such as, but not limited to, synthetic nature-identical MMOs, modified plant polysaccharides, modified animal polysaccharides, or glycans released from animal or plant glycoproteins, support growth and metabolic activities of these bacteria.

The bacteria may be administered contemporaneously with the OS, or they may already be present in the mammalian gut. Unlike most gut flora, certain important bifidobacterial such as, but not limited to, B. longum and B. breve, can internalize oligosaccharides that may be up to 3-20 sugar moieties in length providing that those oligosaccharides have certain specific glycosidic linkages for which these bifidobacteria have endogenous glycosyl hydrolases to deconstruct the oligosaccharides. The functional range may preferably be further limited to 3-10 sugar moieties. This characteristic makes these bifidobacteria uniquely successful in colonizing the gut of the breast-fed infant, the oligosaccharides (denoted herein as MMOs) are the right size and right composition to be uniquely consumed by these bacteria alone. Such structures also found in the carbohydrate components of certain plant and animal glycoproteins. The inventors have also discovered that when these glycans are released from their constituent proteins, they too can be used as a mimic of MMOs. Such oligosaccharides are preferentially internalized and metabolized by such bacteria as a consequence of their unique genetic capacity to do so. The oligosaccharides may be found in mammalian milk, but can also be synthetic or plant-derived as long as they have the ability to select for the specific organism that can provide nutritive components (i.e metabolites) required for the growth and/or development of an infant mammal.

The compositions may be a food composition sufficient to provide partial or total source of nutrition for the mammal. The bacteria and the oligosaccharide, separately or in a food composition, are administered in amounts sufficient to maintain a desired level and composition of at least one metabolite in the mammal. The method can include the steps of: (a) obtaining a fecal or systemic (e.g., urine, plasma) sample from the mammal; (b) determining the level and composition of at least one metabolite in the sample; (c) identifying at least one metabolite insufficiency and/or at least one metabolite excess state in the mammal (i.e., if the level of the metabolite is too high or too low); (d) treating the dysmetabolomic mammal by: (i) administering a bacterial composition comprising bacteria capable of and/or activated for colonization of the intestine; (ii) administering a food composition comprising OS (e.g., MMO or functionally similar oligosaccharide); or (iii) both (i) and (ii) added contemporaneously. This embodiment can provide a method of enhancing the health of a mammal. The bacteria and/or the food composition can be administered in respective amounts sufficient to maintain a level of the subject metabolite in the intestine of the mammal and/or systemically above or below the threshold level related to the excess or insufficiency recited in step (c).

Bacteria Compositions for Use According to this Invention.

The bacteria can be a single bacterial species of Bifidobacterium such as B. adolescentis, B. animalis (e.g., B. animalis subsp. animalis or B. animalis subsp. lactis), B. bifidum, B. breve, B. catenulatum, B. longum (e.g., B. longum subsp. infantis or B. longum subsp. longum), B. pseudocatanulatum, B. pseudolongum, a single bacterial species of Lactobacillus, such as L. acidophilus, L. antri, L. brevis, L. casei, L. coleohominis, L. crispatus, L. curvatus, L. fermentum, L. gasseri, L. johnsonii, L. mucosae, L. pentosus, L. plantarum, L. reuteri, L. rhamnosus, L. sakei, L. salivarius, L. paracasei, L. kisonensis., L. paralimentarius, L. perolens, L. apis, L. ghanensis, L. dextrinicus, L. shenzenensis, L. harbinensis, or a single bacterial species of Pediococcus, such as P. parvulus, P. lolii, P. acidilactici, P. argentinicus, P. claussenii, P. pentosaceus, or P. stilesii, or it can include two or more of any of these species. Typically, at least one of the species will be capable of consuming OS by the internalization of that intact OS within the bacterial cell itself. In a preferred embodiment, the bacterial compositions comprise bifidobacteria. In a more preferred embodiment, the bifidobacteria is B. longum or B. breve. In a particularly preferred embodiment, the B. longum is B. longum subsp. infantis.

For use in this invention, the bacteria may be grown axenically in an anaerobic culture, harvested, and dried using, but not limited to, freeze drying, spray drying, or tunnel drying.

In a preferred embodiment the Bifidobacteria is cultivated in the presence of MMO, whose presence activates the bacteria. In some embodiments, the bacteria composition will include bacteria activated for colonization of the colon. The bacteria may be in an activated state as defined by the expression of genes coding for enzymes or proteins such as, but not limited to, fucosidases, sialidases, extracellular glycan binding proteins, and/or sugar permeases. Such an activated state is produced by the cultivation of the bacteria in a medium comprising a OS prior to the harvest and preservation and drying of the bacteria. Activation of B. infantis is described, for example, in PCT/US2015/057226, the disclosure of which is incorporated herein in its entirety.

Oligosaccharides for Compositions According to this Invention.

Mammalian milk contains a significant quantity of mammalian milk oligosaccharides (MMO) as dietary fiber. For example, in human milk, the dietary fiber is about 15% of total dry mass, or about 15% of the total caloric content. These oligosaccharides comprise sugar residues in a form that is not usable directly as an energy source for the mammalian infant or adult, or for most of the microorganisms in the gut of that mammal.

The term “mammalian milk oligosaccharide” or MMO, as used herein, refers to those indigestible glycans, sometimes referred to as “dietary fiber”, or the carbohydrate polymers that are not hydrolyzed by the endogenous mammalian enzymes in the digestive tract (e.g., the small intestine) of the mammal. Mammalian milks contain a significant quantity of MMO that are not usable directly as an energy source for the milk-fed mammal but may be usable by many of the microorganisms in the gut of that mammal. MMOs can be found as free oligosaccharides (3 sugar units or longer, e.g., 3-20 sugar residues) or they may be conjugated or released from proteins or lipids.

Selective oligosaccharides (OS) as defined here are carbohydrates that are not digested by the mammal and favor the growth of particular bacteria over others. Selective oligosaccharides may be from mammalian milk or fractions thereof, or products of recombinant or natural plants, algae, bacteria, yeast, or of chemical origin provided they induce the desired metabolic profile. OS, as used herein refers to those indigestible sugars of length DP3-DP20 from any source including chemical plant, algae, yeast, bacterial or mammal. Oligosaccharides having the chemical structure of the indigestible oligosaccharides found in any mammalian milk are called OS herein, whether or not they are actually sourced from mammalian milk.

The OS can include one or more of the following structures: N-acetyl lactosamine, lacto N-tetrose (LNT), lacto-N-biose (LNB), Lacto-N-triose, Lacto-N-neotetrose (LNnT), fucosyllactose (2′FL or 3′FL), lacto-N-fucopentose (LNFP), lactodifucotetrose, sialyllactose (SL), di sialyllactone-N-tetrose, 2′-fucosyllactose (2′FL), 3′-sialyllactoseamine, 3′-fucosyllactose (3′FL), 3′-sialyl-3-fucosyllactose, 3′-sialyllactose (3′ SL), 6′-sialyllactosamine, 6′-sialyllactose (6′ SL), difucosyllactose, lacto-N-fucosylpentose I (LNFPI), lacto-N-fucosylpentose II (LNFPII), lacto-N-fucosylpentose III (LNFPIII), lacto-N-fucosylpentose V (LNFPV), sialyllacto-N-tetraose, or derivatives thereof. Trifucosyllacto-N-hexaose (TFLNH), Lacto-N-neohexaose (LNnH), Lacto-N-hexose (LNH), Lacto-N-fucosylpentose III (LNFPIII), MFBLNHIV, and MFBLNHIV.

Oligosaccharide may be classified as having Type I or Type II cores with or without additional sialic acid or fucose residues attached. Lacto-N-biose is a dimer that is a building block for Type I core oligosaccharides. Lacto-N-biose is also described as a (Gal-(1,3)-Beta-GlcNAc), synthesized by enzymes bearing homology to beta-3-galactosyltransferase 1 (B3GALT1) found in the human genome. Examples of Oligosaccharides having a Type I core include Lacto-N-tetrose (LNT). N-acetyl-D-lactosamine, also described as β-D-Gal-(1→4)-D-GlcNAc, is a dimer that is a building block for Type II core oligosaccharides. Lacto-N-neotetrose (LNnT) and lacto-N-fucosylpentose III (LNFPIII) are examples of structures containing type II cores. Lacto-N-triose forms part of both type 1 and type 2 HMOs and also of the glycan moieties of glycoproteins.

In some embodiments, the OS contains a Type I core. In a preferred embodiment of the mixture, the OS contains a type II core. See, e.g., U.S. Pat. Nos. 8,197,872, 8,425,930, and 9,200,091. In some embodiments, the OS comprises a Type I and a Type II core.

The MMO used for this invention can include one or more of fucosylated oligosaccharide structures, such as fucosyllactose (FL) or derivatives of FL including but not limited to, lacto-N-fucopentose (LNFP) and lactodifucotetrose (LDFT),

The MMO used for this invention can include a structure selected from the group: N-acetlylactosamine, Lacto-N-Biose (LNB), lacto-N-tetraose (LNT) and lacto-N-neotetraose (LNnT).

The MMO used for this invention can include sialyllactose (SL) or derivatives of SL such as, but not limited to, 3′ sialyllactose (3 SL), 6′ sialyllactose (6SL), and disialyllacto-N-tetrose (DSLNT).

Mammalian milk can be used as a source of OS. Any of the structures described in this invention can be purified from mammalian milk such as, but not limited to, human milk, bovine milk, goat milk, or horse milk, sheep milk or camel milk, or produced directly by fermentation by yeast, algae or bacteria or chemical synthesis. The composition can further comprise one or more bacterial strains with the ability to grow and divide using any of the above sugars or their derivatives thereof as the sole carbon source. Such bacterial strains may be naturally occurring or genetically modified and selected to grow on the specific OS or their derivatives if they did not naturally grow on those oligosaccharides. Examples may include but not limited to any of the following and their derivatives: 2′FL or 3′FL, LNT or LNnT, 3 SL or 6′ SL.

The MMO can be a mixture fucosyllactose (FL) or derivatives of FL and sialyllactose (SL) or derivatives of SL which are naturally found in mammalian milk such as, but not limited to, human milk, bovine milk, goat milk, and horse milk. The FL and SL or derivatives thereof may be found in a ratio from about 1:10 to 10:1. A formulated mixture of sialidated and fucosylated oligosaccharides may be added to a mixture of LNT or LNnT. Functional equivalents of MMO may include identical molecules produced using recombinant DNA technology as described in Australian Publication No. 2012/257395, Australian Publication No. 2012/232727, and International Publication No. WO 2017/046711.

### Plant Based Polysaccharides

In general, plant fibers are large polysaccharide structures that can only be digested extracellularly by colonic bacteria that excrete certain hydrolases, followed by the ingestion of free sugar monomers or oligosaccharides produced by the extracellular hydrolysis. However, the enzymatic, chemical or biological treatment of plant fibers can reduce the size of the glycans to the size that could be utilized by certain bacterial that are capable of ingesting and deconstructing MMOs such as, but not limited to, B. longum and B. breve. In addition, this invention contemplates treatment by synthetically and/or recombinantly-produced hydrolases that mimic microbial carbohydrate hydrolases, such as GH5, GH13, GH92, GH29 (as described in U.S. Provisional application entitled, “Oligosaccharide Compositions and Their Use During Transitional Phases of the Mammalian Gut Microbiome” filed on even date herewith). Chemical treatment of plant polysaccharides would include acid hydrolysis (sulfuric, hydrochloric, uric, triflouroacetic, etc), or hydrolysis using acidic hydrophobic, non-aqueous, ionic fluids followed by separation of the oligosaccharides in a two phase reaction with water (Kuroda et al., ACS Sustainable Chem. Eng., 2016, 4 (6), pp 3352-3356). Polysaccharides or glycans attached to proteins or lipids can be released by enzymatic processes using N-linked and/or O-linked glycans.

Plant based polysaccharides can be used in the instant invention, if they are first modified to produce a number of different oligosaccharides that closely resemble the majority of HMOs in size (DP 3-10). As such, they can then be used to promote the growth of more beneficial microorganism such as bifidobacteria, lactobacilli and/or pediococci.

Plant-based polysaccharides may come from any conventional or functional foods, such as, but limited to, carrots, peas, onions, and broccoli. Polysaccharides may also come from food processing waste streams including shells, husks, rinds, leaves and clippings from vegetables, fruits, beans and tubers, such as, but not limited to, orange peels, onion hulls, cocao hulls, applecake, grape pomace, pea pods, olive pomace, tomato skins, sugar beets (Mueller-Maatsch et al, Food Chemistry. 2016. 201: 37-45). Sugar beet has β1-3 and β1-4D-glucans (Kuudsen et al. 2007. Br. I. Nutr). They may also come from algae or yeast extracts. The polysaccharide may be part of a mixed food product or a purified polysaccharide fraction. The polysaccharide may be soluble fiber. The polysaccharide may be pre-treated physically, chemically, enzymatically, biologically, with inorganic catalysts, by fermentation, and/or with ionic fluids to convert insoluble fiber to soluble fiber.

The plant-based oligosaccharide composition of this invention can be products produced by enzymatic digestion of the polysaccharide. In some embodiments, the polysaccharides are pre-digested in a controlled fermentation. In some embodiments, the enzyme is cloned, purified and/or immobilized in a process for throughput of the polysaccharide. The released oligosaccharides may by purified or not from the polysaccharide or other components in the food matrix. In some embodiments, the cloned enzyme may be expressed in E. coli or yeast or other suitable organisms such as Bacillus to produce the desired oligosaccharides from the polysaccharide substrate. In some embodiments, an organism containing genes coding for enzymes such as, but not limited to, GH5, GH43, GH13, GH92 are used in a fermentation designed to produce new oligosaccharide. In some embodiments, cellulose is included in the formulation to maintain a certain percentage of insoluble fiber for appropriate bulk and water properties of fecal matter.

In some embodiments, one pot enzymatic reactions are used with multiple endo- and exohydrolases to produce new compositions of oligosaccharides from polysaccharides.

The plant-based oligosaccharide composition of this invention can be produced by chemical breakdown of the polysaccharides by conventional hydrolysis using strong acids such as, but not limited to sulfuric, hydrochloric, uric, and triflouroacetic, under elevated temperatures, followed by neutralization with a strong base such as, but not limited to, NaOH or KOH, and separation and drying of the final oligosaccharides. Inorganic catalysts such as small molecules or mineral ions may be used to reduce chain length or modify oligosaccharide structures (e.g., NaCl, KCl, MgCl₂, CaCl₂, Ca(OH)₂, Ca(NO₃)₂, CaCO₃, or CaHPO₄). The polysaccharides can also be hydrolyzed by exposing the polysaccharides to a catalyst (ionic solvent tolerant enzyme) that selectively cuts glycosidic bonds, in ionic liquid solvents with high thermal stability, low flammability and very low volatility including, but not limited to 1-ethyl-3-methylimidazolium acetate ([EMIM]AcO), 1-allyl-3-methylimidazolium chloride ([AMIM]Cl), 1-butyl-3-methylimidazolium chloride ([BMIM]Cl) and dialkylimidazolium dialkylphosphates (Wahlstrum and Suurankki (2015) Green Chem 17:694). An alternative acid ionic liquid (sulfuric acid and 1-(1-butylsulfonic)-3-methylimidazolium hydrosulfate) has been shown to efficiently hydrolyze polysaccharides in situ at only 100° C. (Satrai, et al, Sustainable Chem. Eng., 2017, 5 (1), pp 708-713) and can also be used in this invention. Oligosaccharides are then removed from the reaction mixture by the addition of water which forms a phase separation with the ionic fluid. In both cases, the reaction is stopped when the predominant oligosaccharide chain length is from DP 3-10.

The plant-based oligosaccharide composition of this invention can alternatively be produced by sonication, and/or heating and/or disruption under pressure to produce oligosaccharide chain length is from DP3-10.

In other embodiments, a combination of one or more techniques to break polysaccharides may be used to create a new product which has a composition that may be defined by LC/MS or other techniques. In further embodiments, the new pool of oligosaccharides of defined chain length are evaluated for their ability to grow specific selected species and/or their lack of ability to promote growth of other organisms.

Glycans attached to proteins from any source (plant, animal or microbial) can be released by an enzymatic process using N-linked and/or O-linked hydrolases and used as a starting point for this invention. Such structures are found in the carbohydrate components of certain plant and animal glycoproteins. These carbohydrates may be longer than desired DP and would be classified as polysaccharides. The inventors have also discovered that when these longer glycans are released from their constituent proteins, they too can be used in the instant invention.

Arabinoxylan is an example of a hemicellulose—a polysaccharide containing arabinose and xylose. Chitin and chitosan are examples of polysaccharides that are inaccessible, but can provide a valuable monomer—N-acetylglucosamine (NAG) or repeating units of NAG that are more accessible to beneficial gut bacteria. In some embodiment, a commensal organism containing the GH46 gene and expresses the enzyme such as P. claussenii is used to facilitate degradation of chitin or chitosan. Other major polysaccharides include components of plant cell walls, such as rhamnogalacturonan, xyloglucan, mannans, glucomannans, pectins, homogalacturonan, and arabinogalacturonans. Other useful polysaccharides include pectin, such as from applecake, cacao hulls, orange peel, sugar beet that have viable compositions and can result in more selectivity compared to other simpler repeating unit polymer compositions. Pectins may include rhamanose, arabinose, fucose, mannose, and xylose.

The above methods for formulating dietary fiber that feed certain populations of bacteria within the microbial food chain can be used with or without the corresponding bacteria to directionally shift the microbiome to establish and/or retain a gut microbiome highly enriched in certain bacterial species within the gut microbiome of a mammal.

In a preferred embodiment, formulations can contain at least 5%, 10%, 15%, 20%, 25%, 30%, 35%, 40%, 45%, 50%, 55%, 60%, 65%, 70%, 75%, 80%, 85%, 90%, or at least 95% of N-acetyl-D-lactosamine (dimer; Type II core typical in LNnT). In other preferred embodiments, formulations that contain at least 5%, 10%, 15%, 20%, 25%, 30%, 35%, 40%, 45%, 50%, 55%, 60%, 65%, 70%, 75%, 80%, 85%, 90%, or at least 95% percentage of Type I core HMO (Gal-(1,3)-Beta-GlcNAc), synthesized by enzymes bearing homology to beta-3-galactosyltransferase 1 (B3GALT1) found in the human genome, can be used. In another preferred embodiment an oligosaccharide not found in human milk, such as a dimer structure or other intermediate dimer, including lacto-N-biose, found during the synthetic production of oligosaccharides can be used. In other preferred embodiments, formulations that contain 5%, 10%, 15%, 20%, 25%, 30%, 35%, 40%, 45%, 50%, 55%, 60%, 65%, 70%, 75%, 80%, 85%, 90%, or at least 95% percentage of lacto-N-triose I (Gal-(1,3)-beta-GlcNAc-(1,3)-Gal), or lacto-N-triose II (GlcNAc-(1,3)-Gal-(1,3)-beta-Glu) or lacto-N-neotriose (Gal-(1,4)-beta-GlcNAc-(1,3)-Gal, can be used. The MMO may provide 0.2 grams to 40 gram per day.

MMO or similar selective oligosaccharides used at percentages above 5%, 10%, 15%, 20%, 25%, 30%, 35%, 40%, 45%, 50%, 55%, 60%, 65%, 70%, 75%, 80%, 85%, 90%, 95% diluted in non-specific carbohydrates such as, but not limited to galactooligosaccharides (GOS), fructoologosaccharides (FOS), Xylosoligosaccharides (XOS) or combinations thereof in percentages below 95%, 90%, 85%, 80%, 75%, 70%, 65%, 60%, 55%, 50%, 45%. 40%, 35%, 30%, 25%, 20%, 15%, 10%, 5%. These combinations provide degrees of increasing selectivity where the higher the proportion of MMO or sources of selective oligosaccharide structures, the greater the selectivity for certain bacteria such as, but not limited to B. longum subsp. infantis.

Modifying the oligosaccharide structure to increase sialylation (sialyllactosamine) or fucosylation can further increase their selectivity. In other embodiments, the formulation contains type II core dimers of lactosamine, and fucosylated and/or sialidated oligosaccharides as the selective carbohydrate fraction; the remainder of which is made up with less selective or non-selective carbohydrates.

The OS may be provided to the mammal directly or in the form of a food composition. The composition can further include a food, and the food can comprise partial or the complete nutritional requirements to support life of a healthy mammal, where that mammal may be, but is not limited to, an infant or adult. The food composition can include mammalian milk, mammalian milk derived product, mammalian donor milk, an infant formula, milk replacer, an enteral nutrition product, or meal replacer for a mammal including a human. The OS may be in the form of a powder or liquid (water-based or oil-based).

Formulations for Compositions According to this Invention.

A composition comprising: (a) bacteria capable of consuming the OS; and (b) one or more OS can be stored as a powder in a low water activity environment for later administration.

The bacteria may be present in these compositions in a dry powder form with water activity less than 0.4, less than 0.3, less than 0.2 or less than 0.1, or as a suspension in a concentrated syrup with a water activity of less than 1.0, preferably less than 0.8, less than 0.6 or less than 0.5, or less than 0.4, or less than 0.3 or less than 0.2 or in a suspension in an oil such as, but not limited to, medium chain triglyceride (MCT), a natural food oil, an algal oil, a fungal oil, a fish oil, a mineral oil, a silicon oil, a phospholipid, or a glycolipid.

The OS can be present in the compositions of this invention in a powder form, in the form of a concentrated syrup with a water activity of less than 1.0, optionally less than 0.9, less than 0.8, less than 0.7, or less than 0.6, or less than 0.5, or less than 0.4, or less than 0.3 or less than 0.2 or in a suspension in an oil including, but not limited to, medium chain triglyceride (MCT), a natural food oil, an algal oil, a fungal oil, a fish oil, a mineral oil, a silicon oil, a phospholipid, and a glycolipid.

The OS composition may be a powder or a concentrate of a MMO such as, but not limited to, that from human milk (HMO), bovine milk (BMO), ovine milk (OMO), equine milk (EMO), or caprine milk (CMO). The oligosaccharides for OS can be obtained from a process that involves cheese or yogurt production and can be from whey sources such as, but not limited to, the whey permeate, or a processed whey permeate, where the processing steps may include, but are not limited to, removal of lactose, removal of minerals, removal of peptides, and removal of monosaccharides, but which in any case, results in the concentration of the OS to levels that are greater than 20%, greater than 30%, greater than 40%, greater than 50%, greater than 60%, greater than 70%, or greater than 80% of the total dry matter of the product.

The composition can be a liquid tonic or a dried powder of the bacterial supernatant containing one or more of the bacterial metabolites from Table 1, Table 2, Table 3 or Table 4. In some embodiments, the bacterial cells are removed. In other embodiments, the bacterial cells are part of the formulation. In some embodiments, the product is a fermented beverage containing at least one desired metabolites from Table 1-5. The fermentation occurs with OS as carbon source to generate a fermentation that contains the desired bacterial metabolites.

The composition can also include a food source that contains all the nutritional requirements to support life of a healthy mammal. That mammal may be, but is not limited to, an infant, an adolescent, an adult, or a geriatric adult. The food source can be a nutritional formulation designed for a human, buffalo, camel, cat, cow, dog, goat, guinea pigs, hamster, horse, pig, rabbit, sheep, monkey, mouse, or rat. For example, the food source can be a food source for an infant human which further comprises a protein such as, but not limited to, a milk protein, a cereal protein, a seed protein, or a tuber protein. The food source can be mammalian milk including, but not limited to, milk from human, bovine, equine, caprine, or porcine sources. The food can also be a medical food or enteral food designed to meet the nutritional requirements for a mammal, for example, a human.

### Methods of Treatment

Metabolites can be delivered directly to the intestine using the composition(s) according to this invention. Any of the compositions described herein can be administered to a mammal to alter the metabolome, which may prevent, modulate or repair gut dysfunction. The mammal may be, but is not limited to, an infant, an adolescent, an adult, or a geriatric adult. The mammal may be a human, buffalo, camel, cat, cow, dog, goat, guinea pig, hamster, horse, pig, rabbit, sheep, monkey, mouse, or rat.

The bacterial and/or the OS compositions described herein can be administered to a mammal to increase the levels of certain metabolites in the gut of the mammal, for example, hippurate, gamma-glutamylcysteine, conjugated primary bile acids, pipecolate, vitamins or their precursors. In some embodiments, increasing benzoic acid detoxification reduces symptoms of jaundice. In other embodiments, benzoic acid toxicity is reduced in infants exposed to bacterial hippurate degraders such as but not limited to Group B strep. In further embodiments, fecal hippurate and benzoic acid are monitored in infants and/or premature infants. In other embodiments, hippurate/benzoic acid ratio is increased in infants at risk for autism or children with autism. In some embodiments, the increase in fecal metabolites facilitate increasing intestinal epithelial barrier function, decreasing bacterial translocation or decreasing leakiness of the intestinal barrier to other metabolites that would show up in urine or systemically.

The bacterial and/or the OS compositions described herein can be administered to a mammal to decrease the levels of certain metabolites in the gut of the mammal, for example, secondary bile acids, dipeptides, benzoic acid or its salts. In other embodiments, excess bile acids are reduced in the colon. In other embodiments, diarrhea is reduced when bile acids are reduced. In other embodiments, the fecal amount of conjugated primary bile acids cholate and chenodeoxycholate are increased and/or secondary bile acids are decreased.

If a mammal is a human infant that has high risk factors for Type I diabetes, then such compositions described herein can be administered to reduce risk of Type I diabetes. If such a mammal is a human infant without autoimmune deficiency risk factors, such compositions described herein can reduce the risk factors for obesity, Type 2 diabetes, Type I diabetes, celiac disease, food allergies, asthma, autism, and atopy. This reduction of risk factors occurs to a much greater extent if the infant is not receiving breast milk.

One or more of the metabolites listed in Table 1 may be increased by at least 5%, 10%, 15%, 20%, 25%, 30%, 35%, 40%, 45%, 50%, 55%, 60%, 65%, 70%, 75%, 80%, 85%, 90%, 95%, 100% compared to the dysmetabolic state. In other embodiments, one or more of the metabolites listed in Table 1 may be decreased by at least 5%, 10%, 15%, 20%, 25%, 30%, 35%, 40%, 45%, 50%, 55%, 60%, 65%, 70%, 75%, 80%, 85%, 90%, 95%, 100% compared to the dysmetabolic state. Alternatively, the metabolite levels may be increased or decreased by 1-fold, 2-fold, 3-fold, 5-fold, 8-fold, 10-fold, 12-fold or 15-fold compared to the dysmetabolic state by administration of the composition of this invention.

One or more of the metabolites listed in Table 1 may be increased by at least 5%, 10%, 15%, 20%, 25%, 30%, 35%, 40%, 45%, 50%, 55%, 60%, 65%, 70%, 75%, 80%, 85%, 90%, 95%, 100% compared to the dysbiotic state. In other embodiments, one or more of the metabolites listed in Table 1 may be reduced by at least 5%, 10%, 15%, 20%, 25%, 30%, 35%, 40%, 45%, 50%, 55%, 60%, 65%, 70%, 75%, 80%, 85%, 90%, 95%, 100% compared to the dysbiotic state.

The mammal whose intestine is colonized with the bacteria described herein may be treated by administering OS (e.g., MMO or other oligosaccharides described herein). The mammal can be a human and/or the bacteria can be a bifidobacteria. The OS can be isolated from, or is chemically identical to, a HMO or a BMO. The OS can comprise N-acetyl-D-lactosamine, LnNT, N-acetyl lactosamine, lacto N-tetrose, lacto-N-biose or fucosyllactose (FL) or derivatives of FL and/or sialyllactose (SL) or as derivatives of SL. The bifidobacteria can be provided as B. longum, for example, B. longum subsp. infantis.

In some embodiments, any of the compositions described herein containing bacteria are provided to the subject on a daily basis comprising from 0.1 billion to 500 billion cfu of bacteria/day. For example, the composition that is provided on a daily basis can include from 1 billion to 100 billion cfu/day or from 5 billion to 20 billion cfu/day. The composition may be provided on a daily basis for at least 2, at least 5, at least 10, at least 20, or at least 30 days. The recipient of the treatment can be a human infant or other mammal.

Any of the bacterial compositions described herein can contain from 0.1 billion to 500 billion cfu of bacteria. Any of the bacterial compositions described herein can be provided on a daily basis. Any of the compositions described herein can comprise from 1 billion to 100 billion cfu, or from 5 billion to 20 billion cfu and can also be provided on a daily basis. The OS can be provided in a solid or liquid form at a dose from about 0.1-50 g/day, for example, 2-30 g/day or 3-10 g/day.

The bacteria can be provided contemporaneously with the OS. In some embodiments, the administration of the bacterial composition and the food composition that includes OS can occur contemporaneously, e.g., within less than 2 hours of each other. In some embodiments, the bacteria can be provided separately to a nursing infant whose OS are in the form of whole milk provided by nursing or otherwise.

The levels of administration of any of the compositions described herein can be altered over time to control the level of certain metabolites in the intestine and/or systemically of the mammal. In some embodiments, the level of OS can be increased or decreased to alter the level of metabolite in the intestinal and/or systematically in the mammal. For example, where colonization of the subject's intestine by bacteria of this invention is associated with increased levels of a metabolite, the level of OS can be increased to increase the level of the metabolite in the intestine of the mammal. The level of OS can be decreased to decrease the level of the metabolite in the intestine of the mammal.

Particular metabolites can be delivered directly to the intestine by administering the compositions described herein. For example, serotonin can be delivered directly to the intestine by administering any of the compositions described herein. Increased levels of serotonin in the colon are beneficial because serotonin in the colon is an important precursor to serotonin elsewhere in the body. Levels of serotonin in a mammal can be increased in the colon by administering any of the OS compositions described herein to the mammal who is receiving or is colonized by one or more species of bacteria according to this invention.

It is beneficial to administer certain metabolites directly to the intestine. For example, increased levels of hippurate in the colon are beneficial because hippurate is detoxifying. Levels of hippurate in a mammal can be increased in the colon by administering any of the OS compositions described herein to the mammal who is receiving or is colonized by one or more species of bacteria according to this invention.

Dysmetabolosis, as targeted for therapy according to this invention, may occur in conjunction with dysbiosis. Generally, the phrase “dysbiosis” is described as the state of microbiome imbalance inside the body, resulting from an insufficient level of keystone bacteria (e.g., bifidobacteria, such as B. longum subsp. infantis) or an overabundance of harmful bacteria in the gut and/or inflammation of the intestine.

Dysbiosis in a human infant is frequently associated with a microbiome that comprises B. longum subsp. infantis below the level of 10⁸ cfu/g fecal material during the first 12 months of life, likely below the level of detectable amount (i.e., less than 10⁶ cfu/g fecal material). Dysbiosis can be further defined as inappropriate diversity or distribution of species abundance for the age of the human or animal. Dysbiosis in infants is driven by either the absence of MMO, absence of B. infantis, or the incomplete or inappropriate breakdown of MMO. For example, in an infant human, an insufficient level of keystone bacteria (e.g., bifidobacteria, such as B. longum subsp. infantis) may be at a level below which colonization of the bifidobacteria in the intestine will not be significant (for example, around 10⁶ cfu/g stool or less). For non-human mammals, dysbiosis can be defined as the presence of members of the Enterobacteraceae family at greater than 10⁶, or 10⁷, or 10⁸ cfu/g feces from the subject mammal. Additionally, a dysbiotic mammal (e.g., a dysbiotic infant) can be defined herein as a mammal having a fecal pH of 6.0 or higher, a watery stool, Clostridium difficile levels of greater than 10⁶ cfu/g feces, greater than 10⁷ cfu/g feces, or greater than 10⁸ cfu/g feces, Enterobacteriaceae at levels of greater than 10⁶, greater than 10⁷, or greater than 10⁸ cfu/g feces, and/or a stool pH of 5.5 or above, 6.0 or above, or 6.5 or above.

Dysbiosis in a mammal, especially an infant mammal, can be observed by the physical symptoms of the mammal (e.g., diarrhea, digestive discomfort such as fussiness excessive crying and colic, inflammation, etc.) and/or by observation of the presence of free sugar monomers in the feces of the mammal, an absence or reduction in specific bifidobacteria populations, and/or the overall reduction in measured SCFA; more specifically, acetate and lactate. Additionally, the infant mammal may have an increased likelihood of becoming dysbiotic based on the circumstances in the environment surrounding the mammal (e.g., an outbreak of disease in the surroundings of the mammal, formula feeding, cesarean birth, etc.). Dysbiosis in an infant mammal can further be revealed by a low level of SCFA in the feces of said mammal. Treatment of dysbiotic mammals is described in International Application No. PCT/US2017/040530, incorporated herein by reference in its entirety. The methods of this invention may be used as an adjunct therapy for dysbiosis.

### EXAMPLES

### Example 1. Trial with Breast-Fed Infants

This trial was designed to show the effect of probiotic supplementation with bifidobacteria in healthy term nursing infants compared to an unsupplemented group. A dry composition of lactose and activated Bifidobacterium longum subsp. infantis was prepared starting with the cultivation of a purified isolate (Strain EVC001, Evolve Biosystems Inc., Davis, Calif., isolated from a human infant fecal sample) in the presence of BMO according to PCT/US2015/057226. The culture was harvested by centrifugation, freeze dried, and the concentrated powder preparation had an activity of about 300 Billion CFU/g. This concentrated powder was then diluted by blending with infant formula grade lactose to an activity level of about 30 Billion CFU/g. This composition then was loaded into individual sachets at about 0.625 g/sachet and provided to breast-fed infants starting on or about day 7 of life and then provided on a daily basis for the subsequent 21 days.

This was a 60-day study starting with infants' date of birth as Day 1. Before postnatal day 6, women and their infants (delivered either vaginally or by cesarean-section), were randomized into an unsupplemented lactation support group or a B. infantis supplementation plus lactation support group. Infant birthweight, birth length, gestational age at birth, and gender were not different between the supplemented and unsupplemented groups. Starting with Day 7 postnatal, and for 21 consecutive days thereafter, infants in the supplemented group were given a dose of at least 1.8×10¹⁰ cfu of B. infantis suspended in 5 mL of their mother's breastmilk, once daily. Because the provision of HMO via breastmilk was critical for supporting the colonization of B. infantis, all participants received breast feeding support at the hospital and at home and maintained exclusive breast feeding through the first 60 days of life.

Infant fecal samples were collected throughout the 60-day trial. Mothers collected their own fecal and breastmilk samples as well as fecal samples from their infants. They filled out weekly, biweekly and monthly health and diet questionnaires, as well as daily logs about their infant feeding and gastrointestinal tolerability (GI). Safety and tolerability was determined from maternal reports of infants' feeding, stooling frequency, and consistency (using a modified Amsterdam infant stool scale—watery, soft, formed, hard; Bekkali et al. 2009), as well as GI symptoms and health outcomes. Individual fecal samples were subjected to full microbiome analysis using Illumina sequencing based on 16S rDNA and qPCR with primers designed specifically for B. longum subsp. infantis strain.

### Results

B. infantis was determined to be well-tolerated. Adverse events reported were events that would be expected in normal healthy term infants and were not different between groups. Reports specifically monitored blood in infant stool, infant body temperature and parental ratings of GI-related infant outcomes such as general irritability, upset feelings in response to spit-ups and discomfort in passing stool or gas, and flatulence. Furthermore, there were no differences reported in the use of antibiotics, gas-relieving medications, or parental report of infant colic, jaundice, number of illnesses, sick doctor visits and medical diagnoses of eczema.

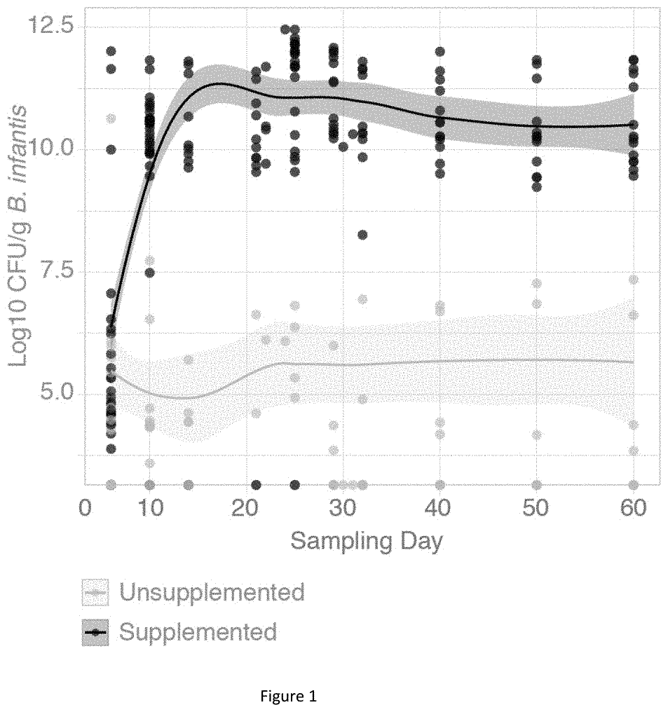

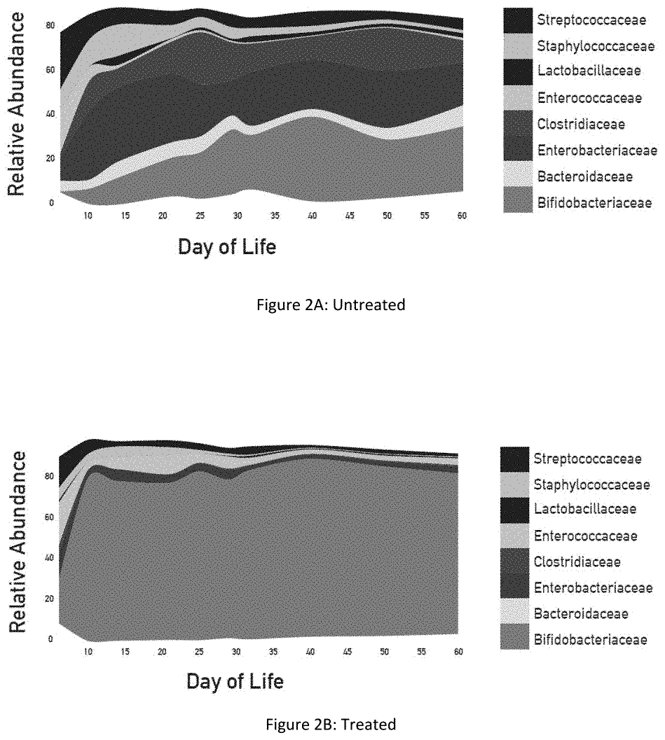

The B. infantis supplemented infants had a gut microbiome fully dominated (on average, greater than 70%) with B. longum subsp. infantis regardless of the birthing mode (vaginal or C-section). This dominance continued even after supplementation ended (Day 28) as long as the infant continued to consume breast milk indicating that B. infantis was colonizing the infant gut to levels higher than 10¹⁰ cfu/g feces (FIG. 1). Furthermore, those infants that were colonized by the B. longum subsp. infantis also had much lower levels of proteobacteria and enterococci (including Clostridium and Escherichia species) (FIGS. 2A and 2B).

Unsupplemented infants (i.e., infants receiving the standard of care—lactation support but no supplementation of B. infantis) did not show B. infantis levels above 10⁶ cfu/g (i.e., the limit of detection) in their microbiome, and there were significant differences in the microbiomes between C-section and vaginally delivered infants. Eighty percent (8 of 10) unsupplemented infants delivered by C-section had no detectable Bifidobacterium species, and fifty-four percent (13 of 24) of the vaginally delivered infants had no detectable Bifidobacterium species by day 60. Further analysis of the thirteen unsupplemented infants that had some detectable bifidobacteria, found that the species were primarily B. longum subsp. longum, B. breve and B. pseudocatenulatum. No detectable B. longum subsp. infantis was found in any of the unsupplemented infants in the study. Further characterization of the stool and other characteristics of supplemented and unsupplemented infants is provided in Example 2 below, and in International Application No. PCT/US2017/040530, incorporated herein by reference.

### Example 2. Metabolomic Analysis of Infant Feces

In Example 1, breast-fed infants received either no supplementation or 21 days of probiotic Bifidobacterium longum subsp. infantis EVC001 (which is genetically similar to the ATCC15697 strain). Fecal samples from infants of Example 1 were evaluated as described below to characterize the fecal metabolome and what effects colonization by this organism may have on the infant's metabolism as a whole.

Sample Preparation: Fecal samples were maintained at −80° C. until processed. Samples were prepared using the automated MicroLab STAR® system from Hamilton Company. Several recovery standards were added prior to the first step in the extraction process for QC purposes. To remove protein, dissociate small molecules bound to protein or trapped in the precipitated protein matrix, and to recover chemically diverse metabolites, proteins were precipitated with methanol under vigorous shaking for 2 min (Glen Mills GenoGrinder 2000) followed by centrifugation. The resulting extract was divided into five fractions: two for analysis by two separate reverse phase (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, and one sample was reserved for backup. Samples were placed briefly on a TurboVap® (Zymark) to remove the organic solvent. The sample extracts were stored overnight under nitrogen before preparation for analysis.

Preparation of study-tracking replicates. A small aliquot of each sample was pooled to create a study tracking sample, which was then injected periodically throughout the platform run. Variability detected in the study tracking sample among consistently detected biochemicals can be used to calculate an estimate of overall process and platform variability.

Ultrahigh Performance Liquid Chromatography-Tandem Mass Spectroscopy (UPLC-MS/MS): All methods utilized a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried then reconstituted in solvents compatible to each of the four methods. Each reconstitution solvent contained a series of standards at fixed concentrations to ensure injection and chromatographic consistency. One aliquot was analyzed using acidic positive ion conditions, chromatographically optimized for more hydrophilic compounds. In this method, the extract was gradient eluted from a C18 column (Waters UPLC BEH C18-2.1×100 mm, 1.7 μm) using water and methanol, containing 0.05% perfluoropentanoic acid (PFPA) and 0.1% formic acid (FA). Another aliquot was also analyzed using acidic positive ion conditions, however it was chromatographically optimized for more hydrophobic compounds. In this method, the extract was gradient eluted from the same afore mentioned C18 column using methanol, acetonitrile, water, 0.05% PFPA and 0.01% FA and was operated at an overall higher organic content. Another aliquot was analyzed using basic negative ion optimized conditions using a separate dedicated C18 column. The basic extracts were gradient eluted from the column using methanol and water, however with 6.5 mM Ammonium Bicarbonate at pH 8. The fourth aliquot was analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis alternated between MS and data-dependent MS' scans using dynamic exclusion. The scan range varied slighted between methods but covered 70-1000 m/z.

Data Extraction and Compound Identification: Raw data was extracted, peak-identified and QC processed using Proprietary hardware and software. Compounds were identified by comparison to library entries of purified standards or recurrent unknown entities in a library based on authenticated standards that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library +/−10 ppm, and the MS/MS forward and reverse scores between the experimental data and authentic standards. The MS/MS scores are based on a comparison of the ions present in the experimental spectrum to the ions present in the library spectrum.

Metabolite Quantification and Data Normalization: Peaks were quantified using area-under-the-curve. For studies spanning multiple days, a data normalization step was performed to correct variation resulting from instrument inter-day tuning differences. Essentially, each compound was corrected in run-day blocks by registering the medians to equal one (1.00) and normalizing each data point proportionately (termed the “block correction”; FIG. 2). For studies that did not require more than one day of analysis, no normalization is necessary, other than for purposes of data visualization.

Determining the absolute concentration of metabolites in a fecal sample: Once the fecal samples was analyzed for the breadth of metabolites that changed in dysmetabolic infant fecal samples compared to the fecal samples taken from an infant treated with a composition from this invention, a series of known standards were assembled to help determine the absolute concentrations of certain metabolites using liquid chromatography-QTRAP or gas chromatography-quadrupole mass spectrometry. A standard curve is generated for known concentrations of a metabolite using the identified standards and the standard curve is used to determine the concentration of the metabolite in the fecal samples.

### Results

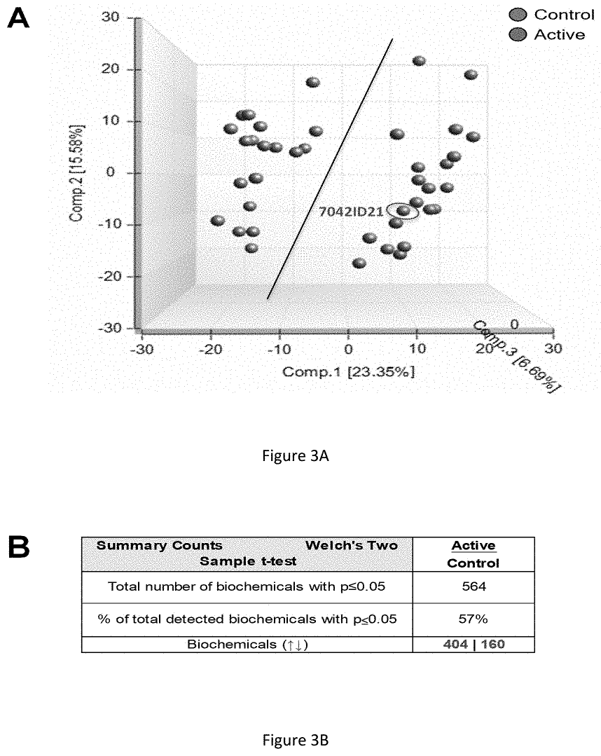

Fecal Samples from 20 infants supplemented with B. longum subsp. infantis (intervention) and 20 infants that were not supplemented (control) were analyzed for the levels of 983 metabolites. Analysis of 983 detected metabolites generated the major findings indicated in the following Table. With the exception of one sample, a clear separation can be observed between the supplemented and unsupplemented samples, pointing toward a substantial metabolic difference between the two groups. (See FIG. 3A, showing the results of Principle component analysis (PCA) for these samples.) Around 57% of the 983 total detected biochemicals were significantly different (p≤0.05) between the two groups, most of which were elevated in the supplemented group (FIG. 3B). By PCA, it was determined that the one sample was an outlier and after further examination, it was determined to be a sample containing high Bifidobacterium level.

A list of metabolites and the ratio of their average levels between two populations is shown in Table 1 with and without the outlier removed.

TABLE 1
Relative abundance of various metabolites.

| | | | | | Int/Con | P-value | |
|---|---|---|---|---|---|---|---|
| Metabolic | | | P-value | Q-value | (outlier | (Int/Con outlier | |
| Pathway | Metabolite | Int/Con | (Int/Con) | (Int/Con) | removed | removed | Q-value |
| Glycine | glycine | 1.56 | 0.258843609 | 0.093907383 | 1.73 | 0.156788547 | 0.058619349 |
| and Serine | N-acetylglycine | 0.39 | 4.4265E−07 | 1.29254E−06 | 0.39 | 1.26309E−06 | 2.40769E−06 |
| Metabolism | sarcosine | 0.1 | 0.300790553 | 0.106296931 | 0.1 | 0.264058157 | 0.092152272 |
| | dimethylglycine | 0.87 | 0.993916757 | 0.263251991 | 0.84 | 0.989686347 | 0.255134788 |
| | betaine | 0.67 | 0.005265697 | 0.003461945 | 0.66 | 0.006325949 | 0.003798861 |
| | serine | 1.79 | 0.001120968 | 0.000915909 | 1.81 | 0.000969505 | 0.0007506 |
| | N-acetylserine | 1.91 | 0.000735093 | 0.000633119 | 2.18 | 0.000120671 | 0.000118605 |
| | 2-methylserine | 1.94 | 0.000184783 | 0.000189191 | 1.92 | 0.000291565 | 0.000261078 |
| | O-acetylhomoserine | 0.41 | 0.399552764 | 0.134343002 | 0.62 | 0.176406234 | 0.064988266 |
| Alanine and | alanine | 1.88 | 0.00027009 | 0.00026587 | 2.12 | 2.75021E−05 | 3.42575E−05 |
| Aspartate | N-acetylalanine | 1.64 | 0.010916736 | 0.006426887 | 1.8 | 0.005771671 | 0.003516359 |
| Metabolism | aspartate | 2.38 | 0.000104146 | 0.000113829 | 2.68 | 2.37294E−05 | 3.08248E−05 |
| | N-acetylaspartate | 0.3 | 0.8769886 | 0.239885764 | 0.29 | 0.858567902 | 0.229576139 |
| | (NAA) | | | | | | |
| | asparagine | 3.24 | 6.77211E−05 | 7.93766E−05 | 3.31 | 6.8938E−05 | 7.41284E−05 |
| | N-acetylasparagine | 2.79 | 3.57046E−06 | 6.80057E−06 | 3.22 | 6.3031E−07 | 1.3791E−06 |
| Glutamate | glutamate | 3.12 | 2.0879E−07 | 6.7606E−07 | 3.27 | 1.3151E−07 | 3.558E−07 |
| Metabolism | glutamine | 2.49 | 0.001141684 | 0.000927077 | 2.38 | 0.00203315 | 0.001428987 |
| | N-acetylglutamate | 1.85 | 0.000556785 | 0.00050079 | 1.86 | 0.000702952 | 0.00057427 |
| | N-acetylglutamine | 1.6 | 0.001124395 | 0.000915909 | 1.86 | 0.000238744 | 0.000216868 |
| | 4-hydroxyglutamate | 1.16 | 0.576207208 | 0.176785257 | 1.23 | 0.447414312 | 0.139259649 |
| | glutamate, gamma- | 7.05 | 2.8E−10 | 3.88E−09 | 12.91 | 4.17E−13 | 9.54E−12 |
| | methyl ester | | | | | | |
| | pyroglutamine* | 0.6 | 0.12587902 | 0.050790072 | 0.58 | 0.099973288 | 0.039865419 |
| | beta-citrylglutamate | 1.43 | 0.054737039 | 0.024831657 | 1.41 | 0.062195201 | 0.026479581 |
| | carboxyethyl-GABA | 1.17 | 0.185658109 | 0.071142272 | 1.2 | 0.137362222 | 0.052130933 |
| | S-1-pyrroline-5- | 0.91 | 0.978388335 | 0.260470733 | 0.88 | 0.905143282 | 0.239737212 |
| | carboxylate | | | | | | |
| | propionylglutamine | 29.19 | 5.31E−13 | 3E−11 | 36.23 | 1.26E−13 | 3.96E−12 |
| | succinylglutamine | 0.95 | 0.852233051 | 0.234850249 | 0.97 | 0.9563539 | 0.250141231 |
| Histidine | histidine | 2.97 | 0.153228417 | 0.060321749 | 3.13 | 0.120321859 | 0.046648916 |
| Metabolism | 1-methylhistidine | 2.09 | 0.000395837 | 0.000372859 | 2.23 | 0.000229685 | 0.000209394 |
| | 3-methylhistidine | 4.32 | 0.004101745 | 0.002803432 | 4.33 | 0.002744032 | 0.001856043 |
| | N-acetylhistidine | 1.4 | 0.966420366 | 0.258346631 | 1.39 | 0.935237212 | 0.245896056 |
| | N-acetyl-3- | 1.27 | 0.405295331 | 0.135699392 | 1.22 | 0.482605139 | 0.147907606 |
| | methylhistidine* | | | | | | |
| | N-acetyl-1- | 2.57 | 0.000192694 | 0.000194979 | 2.66 | 0.000126236 | 0.000123114 |
| | methylhistidine* | | | | | | |
| | trans-urocanate | 0.79 | 0.68029395 | 0.19866866 | 0.77 | 0.65706366 | 0.185138914 |
| | imidazole propionate | 0.48 | 0.002697387 | 0.001951734 | 0.46 | 0.001692704 | 0.001222116 |
| | formiminoglutamate | 2.72 | 0.410129933 | 0.136724208 | 2.59 | 0.293662586 | 0.099583282 |
| | imidazole lactate | 1.34 | 0.061444879 | 0.027300943 | 1.3 | 0.061864671 | 0.0263835 |
| | carnosine | 2.51 | 8.68886E−06 | 1.45208E−05 | 2.97 | 1.43218E−06 | 2.59253E−06 |
| | histamine | 0.83 | 0.186317054 | 0.071289316 | 0.9 | 0.282495603 | 0.096840574 |
| | 1- | 0.94 | 0.634344027 | 0.189306548 | 0.93 | 0.594320882 | 0.170904957 |
| | methylimidazoleacetate | | | | | | |
| | 4-imidazoleacetate | 0.4 | 0.01692774 | 0.009429888 | 0.39 | 0.014428262 | 0.007724278 |
| | N-acetylhistamine | 3.12 | 0.071554484 | 0.031099335 | 5.98 | 0.114969965 | 0.044920052 |
| Lysine | lysine | 2.27 | 0.001760097 | 0.001348902 | 2.42 | 0.000812299 | 0.000650921 |
| Metabolism | N2-acetyllysine | 4.33 | 1.17259E−06 | 2.80273E−06 | 5.33 | 2.5358E−07 | 6.2686E−07 |
| | N6-acetyllysine | 10.71 | 6.21E−09 | 3.895E−08 | 19.9 | 1.3E−10 | 9.9E−10 |
| | N6-formyllysine | 25.12 | 7.3444E−07 | 2.00259E−06 | 29.84 | 4.6165E−07 | 1.04647E−06 |
| | N6,N6,N6- | 2.24 | 1.14939E−05 | 1.81544E−05 | 2.55 | 8.2868E−07 | 1.73759E−06 |
| | trimethyllysine | | | | | | |
| | 5-hydroxylysine | 1.91 | 0.087957112 | 0.03741228 | 1.89 | 0.095043952 | 0.038324952 |
| | saccharopine | 2.76 | 0.000503957 | 0.000461242 | 2.7 | 0.000687141 | 0.000563182 |
| | 2-aminoadipate | 1.47 | 0.133826907 | 0.05341451 | 1.53 | 0.099106371 | 0.039645386 |
| | glutarylcarnitine | 1.68 | 0.004776967 | 0.003189195 | 1.63 | 0.007828261 | 0.004520744 |
| | (C5-DC) | | | | | | |
| | pipecolate | 13.71 | 0.000142457 | 0.000150619 | 15.57 | 7.76577E−05 | 8.1417E−05 |
| | cadaverine | 0.56 | 0.000497577 | 0.000457058 | 0.57 | 0.00077273 | 0.000623182 |
| | N-acetyl-cadaverine | 1.84 | 0.002139982 | 0.001606762 | 1.96 | 0.002851192 | 0.001918212 |
| | 5-aminovalerate | 0.27 | 0.016334713 | 0.009154575 | 0.26 | 0.01551711 | 0.008156592 |
| | N-trimethyl 5- | 1.6 | 0.012253014 | 0.007068986 | 1.66 | 0.008248803 | 0.004717153 |
| | aminovalerate | | | | | | |
| Phenylalanine | phenylalanine | 1.24 | 0.336263214 | 0.116294198 | 1.24 | 0.310877709 | 0.104296574 |
| Metabolism | N-acetylphenylalanine | 0.68 | 0.171967616 | 0.066288456 | 0.92 | 0.290715965 | 0.098984266 |
| | phenylpyruvate | 0.47 | 0.040322286 | 0.019378354 | 0.47 | 0.053129723 | 0.02319786 |
| | phenyllactate (PLA) | 4.18 | 0.00031197 | 0.000299302 | 4.93 | 0.000105416 | 0.000105384 |
| | phenethylamine | 0.84 | 0.928473407 | 0.24974896 | 0.81 | 0.888581095 | 0.236345673 |
| | phenylacetate | 0.3 | 0.012090108 | 0.00700621 | 0.29 | 0.010918966 | 0.006038261 |
| | 4- | 0.7 | 0.255971386 | 0.092995599 | 0.68 | 0.265011524 | 0.092356887 |
| | hydroxyphenylacetate | | | | | | |
| Tyrosine | tyrosine | 1.24 | 0.406364008 | 0.135823225 | 1.23 | 0.420243256 | 0.132507232 |
| Metabolism | N-acetyltyrosine | 1.6 | 0.083460027 | 0.035734166 | 1.68 | 0.057921328 | 0.025041322 |
| | tyramine | 0.63 | 0.033485229 | 0.016519147 | 0.61 | 0.044135985 | 0.019795737 |
| | 4- | 0.24 | 0.006278987 | 0.004045983 | 0.24 | 0.007833479 | 0.004520744 |
| | hydroxyphenylpyruvate | | | | | | |
| | 3-(4- | 8.24 | 5.9E−10 | 5.86E−09 | 16.7 | 1.16E−12 | 2E−11 |
| | hydroxyphenyl)lactate | | | | | | |
| | phenol sulfate | 0.42 | 0.177729011 | 0.068306013 | 0.4 | 0.149923929 | 0.056387983 |
| | vanillactate | 2.64 | 0.000585725 | 0.000524997 | 2.66 | 0.000545198 | 0.000461891 |
| | vanillylmandelate | 1.09 | 0.436806353 | 0.142504622 | 1.06 | 0.491080998 | 0.149775543 |
| | (VMA) | | | | | | |
| | o-Tyrosine | 5.39 | 0.001068587 | 0.000881538 | 5.3 | 0.001231563 | 0.000924437 |
| | dopamine 3-O-sulfate | 2.44 | 3.3997E−06 | 6.67155E−06 | 2.46 | 4.58021E−06 | 7.2482E−06 |
| | tyramine O-sulfate | 2.62 | 0.192088528 | 0.072958779 | 2.62 | 0.192218297 | 0.070401143 |
| | N- | 2.94 | 0.004831133 | 0.003210059 | 2.95 | 0.004501165 | 0.002852831 |
| | formylphenylalanine | | | | | | |
| Tryptophan | tryptophan | 1.15 | 0.683004312 | 0.199013341 | 1.15 | 0.670923895 | 0.187782575 |
| Metabolism | N-acetyltryptophan | 0.72 | 0.129300974 | 0.051847734 | 0.74 | 0.173794044 | 0.064119811 |
| | C-glycosyltryptophan | 1.67 | 0.00736543 | 0.004608486 | 1.72 | 0.006753121 | 0.004007564 |
| | tryptophan betaine | 1.46 | 0.079249987 | 0.034271482 | 1.48 | 0.067500709 | 0.028166491 |
| | kynurenine | 1.65 | 0.000138609 | 0.000147151 | 1.65 | 0.000221924 | 0.000203796 |
| | kynurenate | 1.59 | 0.004419447 | 0.002981242 | 1.57 | 0.006485623 | 0.003874972 |
| | N-formylanthranilic | 0.48 | 0.031555084 | 0.015840911 | 0.46 | 0.029721824 | 0.014137138 |
| | acid | | | | | | |
| | xanthurenate | 1.56 | 0.60984989 | 0.184117966 | 1.48 | 0.837973769 | 0.224786025 |
| | picolinate | 1.81 | 0.006916129 | 0.004358949 | 1.92 | 0.003952353 | 0.002569724 |
| | serotonin | 3.03 | 1.81847E−06 | 3.9584E−06 | 3.58 | 2.6966E−07 | 6.4954E−07 |
| | 5- | 1.23 | 0.20486213 | 0.077244124 | 1.18 | 0.288706285 | 0.098433199 |
| | hydroxyindoleacetate | | | | | | |
| | tryptamine | 1.24 | 0.541404466 | 0.168717181 | 1.18 | 0.618043402 | 0.176917919 |
| | indolelactate | 7.38 | 0.000000059 | 2.3156E−07 | 10.04 | 1.588E−08 | 5.963E−08 |
| | indoleacetate | 0.08 | 5.89121E−05 | 7.19829E−05 | 0.08 | 0.00011874 | 0.000117165 |
| | 3-indoxyl sulfate | 1.41 | 0.330380822 | 0.114719305 | 1.36 | 0.299280523 | 0.100944195 |
| | 2-aminophenol | 0.66 | 0.018203847 | 0.010011583 | 0.66 | 0.025997372 | 0.012677145 |
| Leucine, | leucine | 1.15 | 0.413390831 | 0.137276827 | 1.17 | 0.350735803 | 0.114612266 |
| Isoleucine | N-acetylleucine | 3.06 | 6.1765E−06 | 1.06662E−05 | 3.59 | 9.4183E−07 | 1.91115E−06 |
| and Valine | 4-methyl-2- | 0.83 | 0.650318045 | 0.192521066 | 0.83 | 0.690388969 | 0.191949501 |
| Metabolism | oxopentanoate | | | | | | |
| | alpha- | 0.6 | 0.004548647 | 0.003060427 | 0.6 | 0.005853011 | 0.003557302 |
| | hydroxyisocaproate | | | | | | |
| | isovalerylcarnitine | 3.85 | 6.40101E−06 | 1.09808E−05 | 3.83 | 9.78357E−06 | 1.4396E−05 |
| | (C5) | | | | | | |
| | beta- | 1.08 | 0.886244713 | 0.241397988 | 1.08 | 0.864965873 | 0.231038789 |
| | hydroxyisovalerate | | | | | | |
| | isoleucine | 1.11 | 0.967584646 | 0.258391213 | 1.13 | 0.951475365 | 0.249633282 |
| | N-acetylisoleucine | 0.75 | 0.461623966 | 0.148358971 | 0.78 | 0.570231259 | 0.16576167 |
| | 3-methyl-2- | 0.81 | 0.506043545 | 0.159094011 | 0.81 | 0.540903749 | 0.161448547 |
| | oxovalerate | | | | | | |
| | alpha- | 0.35 | 9.41E−08 | 3.4823E−07 | 0.35 | 9.102E−08 | 2.5734E−07 |
| | hydroxyisovalerate | | | | | | |
| | 2- | 3.3 | 1.15935E−05 | 1.82008E−05 | 3.41 | 9.15677E−06 | 1.3553E−05 |
| | methylbutyrylcarnitine | | | | | | |
| | (C5) | | | | | | |
| | 2- | 0.7 | 0.34982565 | 0.120319218 | 0.69 | 0.375119184 | 0.12091036 |
| | methylbutyrylglycine | | | | | | |
| | tiglylcarnitine | 2.42 | 0.00065314 | 0.000581399 | 2.44 | 0.000633211 | 0.000525833 |
| | (C5:1-DC) | | | | | | |
| | ethylmalonate | 1.11 | 0.360981327 | 0.123076083 | 1.08 | 0.370524244 | 0.120297513 |
| | methylsuccinate | 0.14 | 0.048456313 | 0.022258902 | 0.14 | 0.062491249 | 0.026515891 |
| | valine | 2.12 | 0.003986677 | 0.002739241 | 2.41 | 0.001194086 | 0.000900096 |
| | N-acetylvaline | 2.9 | 1.38834E−05 | 2.12353E−05 | 3.58 | 1.30833E−06 | 2.42058E−06 |
| | 3-methyl-2- | 1.52 | 0.042347686 | 0.020017491 | 1.67 | 0.020407726 | 0.010311147 |
| | oxobutyrate | | | | | | |
| | 2-hydroxy-3- | 0.32 | 9.51E−09 | 5.133E−08 | 0.31 | 8.39E−09 | 3.577E−08 |
| | methylvalerate | | | | | | |
| | isobutyrylcarnitine | 3 | 0.000358972 | 0.000341642 | 3.08 | 0.000298494 | 0.000265394 |
| | (C4) | | | | | | |
| | 3-hydroxyisobutyrate | 1.78 | 0.02507816 | 0.013150105 | 1.81 | 0.020577558 | 0.010376121 |
| | 2,3-dihydroxy-2- | 0.35 | 0.003289304 | 0.002340794 | 0.34 | 0.004295438 | 0.002764219 |
| | methylbutyrate | | | | | | |
| Methionine, | methionine | 1.14 | 0.286760886 | 0.101895004 | 1.15 | 0.251577829 | 0.088850502 |
| Cysteine, | N-acetylmethionine | 2.59 | 1.04726E−05 | 1.70615E−05 | 2.66 | 1.15406E−05 | 1.64236E−05 |
| SAM and | N-formylmethionine | 1.42 | 0.821874332 | 0.229166013 | 1.36 | 0.626149794 | 0.178390565 |
| Taurine | methionine sulfoxide | 0.96 | 0.643687072 | 0.191653185 | 1 | 0.811345737 | 0.218651525 |
| Metabolism | N-acetylmethionine | 0.62 | 0.242108458 | 0.088455376 | 0.62 | 0.292203501 | 0.099222218 |
| | sulfoxide | | | | | | |
| | cystathionine | 2.12 | 1.8464E−05 | 2.676E−05 | 4.63 | 6.965E−08 | 2.0619E−07 |
| | cysteine | 0.86 | 0.398016233 | 0.134071032 | 0.85 | 0.373748725 | 0.120721322 |
| | N-acetylcysteine | 11.83 | 7.136E−08 | 2.7183E−07 | 13.68 | 2.574E−08 | 8.997E−08 |
| | S-methylcysteine | 0.89 | 0.860661883 | 0.236669434 | 0.9 | 0.962897175 | 0.251477134 |
| | cysteine s-sulfate | 1.15 | 0.555789726 | 0.171188673 | 1.18 | 0.642370189 | 0.182017915 |
| | cystine | 0.23 | 0.000389683 | 0.000368402 | 0.27 | 0.000795083 | 0.000639161 |
| | taurine | 3.98 | 2.12221E−05 | 3.03587E−05 | 4.87 | 4.26143E−06 | 6.82963E−06 |
| | N-acetyltaurine | 1.53 | 0.910090955 | 0.245825368 | 1.54 | 0.963460275 | 0.251477134 |
| | 3-sulfo-L-alanine | 5.51 | 0.004289173 | 0.002916145 | 5.51 | 0.004289173 | 0.002764219 |
| Urea cycle; | arginine | 1.93 | 1.59431E−06 | 3.52978E−06 | 1.93 | 2.52508E−06 | 4.26413E−06 |
| Arginine | argininosuccinate | 2.12 | 0.000101543 | 0.000111454 | 2.25 | 7.23028E−05 | 7.67624E−05 |
| and Proline | urea | 1.53 | 0.834146588 | 0.231590766 | 1.74 | 0.663215216 | 0.186454629 |
| Metabolism | ornithine | 2.23 | 0.019474847 | 0.010511586 | 2.56 | 0.008585518 | 0.004875734 |
| | 2-oxoarginine* | 0.77 | 0.498011728 | 0.157138227 | 0.77 | 0.561132711 | 0.164367594 |
| | citrulline | 2.74 | 5.71294E−06 | 1.00671E−05 | 2.89 | 3.3187E−06 | 5.49371E−06 |
| | homocitrulline | 1.2 | 0.23051837 | 0.084698736 | 1.15 | 0.272696683 | 0.094381563 |
| | proline | 1.68 | 0.069180709 | 0.030219747 | 1.84 | 0.035087385 | 0.016199299 |
| | dimethylarginine | 2.3 | 0.000508424 | 0.000462106 | 2.55 | 0.000174492 | 0.00016568 |
| | (SDMA + ADMA) | | | | | | |
| | N-acetylarginine | 2.31 | 8.76071E−05 | 9.90979E−05 | 2.64 | 2.92777E−05 | 3.59355E−05 |
| | N-acetylcitrulline | 4 | 5.1635E−06 | 9.28842E−06 | 5.64 | 5.0163E−07 | 1.12695E−06 |
| | N-acetylproline | 6.47 | 0.000164489 | 0.000170435 | 9.01 | 4.80585E−05 | 5.50115E−05 |
| | N-delta- | 1.72 | 0.000450925 | 0.000420165 | 3.09 | 2.6037E−05 | 3.32557E−05 |
| | acetylornithine | | | | | | |
| | N-alpha- | 4.4 | 7.855E−07 | 2.11951E−06 | 4.86 | 4.1733E−07 | 9.6338E−07 |
| | acetylornithine | | | | | | |
| | trans-4- | 1.61 | 0.002599686 | 0.001907685 | 1.64 | 0.002319556 | 0.00159465 |
| | hydroxyproline | | | | | | |
| | pro-hydroxy-pro | 0.53 | 0.011007055 | 0.006465365 | 0.52 | 0.013943991 | 0.007480937 |
| | N- | 2.73 | 1.05772E−05 | 1.70916E−05 | 3.01 | 3.63374E−06 | 5.97591E−06 |
| | monomethylarginine | | | | | | |
| | argininate* | 0.49 | 0.006503094 | 0.004159351 | 0.48 | 0.007370147 | 0.004302695 |
| Creatine | guanidinoacetate | 0.86 | 0.057797989 | 0.025940173 | 0.83 | 0.045642286 | 0.020254711 |
| Metabolism | creatine | 1.16 | 0.11399534 | 0.046649167 | 1.18 | 0.105734317 | 0.041963184 |
| | creatinine | 1.4 | 0.020913469 | 0.011146803 | 1.41 | 0.021567567 | 0.010788843 |
| | N-methylhydantoin | 0.54 | 0.119833713 | 0.048577892 | 0.53 | 0.119818562 | 0.046525476 |
| Polyamine | agmatine | 0.56 | 0.002641116 | 0.001931649 | 1.05 | 0.004449805 | 0.00283456 |
| Metabolism | putrescine | 0.6 | 4.28983E−06 | 0.000007881 | 0.62 | 5.35656E−06 | 8.42379E−06 |
| | spermidine | 0.05 | 6.38E−09 | 3.895E−08 | 0.05 | 4.52E−09 | 2.147E−08 |
| | acisoga | 1.03 | 0.626521408 | 0.187967273 | 1.03 | 0.58034728 | 0.167653074 |
| | N(1)-acetylspermine | 0.6 | 0.844220755 | 0.233569502 | 0.64 | 0.979411229 | 0.254296682 |
| | N1,N12- | 1.83 | 0.037105017 | 0.018066811 | 1.88 | 0.027969775 | 0.013560114 |
| | diacetylspermine | | | | | | |
| | 5- | 1.13 | 0.004311873 | 0.002923904 | 1.66 | 0.000654425 | 0.000539886 |
| | methylthioadenosine | | | | | | |
| | (MTA) | | | | | | |
| | N-acetylputrescine | 3.37 | 0.045298848 | 0.021142419 | 3.91 | 0.06422738 | 0.027069959 |
| | 1,3-diaminopropane | 0.11 | 0.000198809 | 0.000200384 | 0.1 | 0.00016847 | 0.000160569 |
| | 4- | 2.31 | 0.014909364 | 0.008488056 | 2.46 | 0.008622998 | 0.004875734 |
| | acetamidobutanoate | | | | | | |
| | (N(1) + N(8))- | 0.13 | 0.000100476 | 0.000110753 | 0.13 | 0.000162975 | 0.000157117 |
| | acetylspermidine | | | | | | |
| Guanidino | 1-methylguanidine | 4.08 | 1.47463E−05 | 2.22135E−05 | 4.17 | 1.15532E−05 | 1.64236E−05 |
| and Acetamido | 4-guanidinobutanoate | 0.78 | 0.169467816 | 0.065662786 | 0.95 | 0.276251382 | 0.095372564 |
| Metabolism | guanidinosuccinate | 1.8 | 0.006347504 | 0.004079984 | 1.84 | 0.005067559 | 0.003138182 |
| Glutathione | cysteinylglycine | 1.01 | 0.033684688 | 0.016525671 | 0.99 | 0.042370859 | 0.019072042 |
| Metabolism | 5-oxoproline | 7.66 | 6E−11 | 1.27E−09 | 8.35 | 4E−11 | 3.8E−10 |
| | 2-hydroxybutyrate/2- | 1.3 | 0.153766921 | 0.060441886 | 1.31 | 0.135936992 | 0.051667969 |
| | hydroxyisobutyrate | | | | | | |
| Gamma- | gamma- | 4.52 | 5.0668E−07 | 1.4423E−06 | 5.86 | 7.532E−08 | 2.1784E−07 |
| glutamyl | glutamylalanine | | | | | | |
| Amino Acid | gamma- | 28.92 | 2E−11 | 4.6E−10 | 44.28 | 1.17E−12 | 2E−11 |
| | glutamylcysteine | | | | | | |
| | gamma- | 3.15 | 0.004574788 | 0.003062108 | 3.07 | 0.004279002 | 0.002764219 |
| | glutamylglutamate | | | | | | |
| | gamma- | 1.58 | 0.002680836 | 0.001950657 | 1.64 | 0.001325298 | 0.000975055 |
| | glutamylglutamine | | | | | | |
| | gamma- | 10.16 | 9.2123E−05 | 0.000103753 | 10.47 | 6.88631E−05 | 7.41284E−05 |
| | glutamylhistidine | | | | | | |
| | gamma- | 6.69 | 0.00000242 | 5.09488E−06 | 8.14 | 8.144E−07 | 1.72199E−06 |
| | glutamylisoleucine* | | | | | | |
| | gamma- | 1.59 | 0.029559363 | 0.01501362 | 1.61 | 0.025584145 | 0.012539193 |
| | glutamylleucine | | | | | | |
| | gamma-glutamyl- | 3.28 | 0.056351653 | 0.025342221 | 3.3 | 0.052765195 | 0.023089867 |
| | alpha-lysine | | | | | | |
| | gamma-glutamyl- | 2.26 | 4.53004E−05 | 5.78052E−05 | 2.4 | 1.66338E−05 | 2.26236E−05 |
| | epsilon-lysine | | | | | | |
| | gamma- | 10.82 | 3.679E−08 | 1.6154E−07 | 19.26 | 8.75E−09 | 3.669E−08 |
| | glutamylmethionine | | | | | | |
| | gamma- | 4.21 | 5.96624E−05 | 7.23162E−05 | 4.24 | 5.67663E−05 | 6.32012E−05 |
| | glutamylphenylalanine | | | | | | |
| | gamma- | 2.81 | 0.000510343 | 0.000462229 | 2.75 | 0.000714584 | 0.000581884 |
| | glutamyltyrosine | | | | | | |
| | gamma-glutamylvaline | 3.11 | 2.13302E−05 | 3.03587E−05 | 3.34 | 1.11065E−05 | 1.59691E−05 |
| Dipeptide | N-acetylcarnosine | 2.71 | 0.001093685 | 0.000899378 | 2.85 | 0.000662782 | 0.000544993 |
| Derivative | anserine | 2.18 | 5.32666E−06 | 9.51584E−06 | 2.37 | 1.57429E−06 | 2.82943E−06 |
| Dipeptide | alanylleucine | 3.27 | 9.30819E−06 | 1.53577E−05 | 3.47 | 6.24189E−06 | 9.63542E−06 |
| | glutaminylleucine | 0.9 | 0.357927945 | 0.122478606 | 0.89 | 0.382210004 | 0.122823653 |
| | glycylisoleucine | 0.26 | 6.47E−09 | 3.895E−08 | 0.25 | 6E−11 | 5E−10 |
| | glycylleucine | 0.86 | 0.486977094 | 0.154778784 | 0.84 | 0.424632083 | 0.133389612 |
| | glycylvaline | 0.46 | 2.2338E−07 | 7.0565E−07 | 0.45 | 5.219E−08 | 1.6623E−07 |
| | histidylalanine | 0.35 | 0.00411701 | 0.00280646 | 0.33 | 0.001938797 | 0.001368479 |
| | isoleucylglycine | 0.24 | 4.4409E−07 | 1.29254E−06 | 0.23 | 1.5798E−07 | 4.0979E−07 |
| | leucylalanine | 0.43 | 0.006715146 | 0.0042426 | 0.41 | 0.002495891 | 0.001706552 |
| | leucylglycine | 0.52 | 0.023486153 | 0.012365375 | 0.5 | 0.016283617 | 0.008500521 |
| | lysylleucine | 2.9 | 1.21541E−06 | 2.81103E−06 | 3.05 | 6.7237E−07 | 1.44599E−06 |
| | phenylalanylalanine | 0.47 | 0.008904612 | 0.005414594 | 0.45 | 0.006395421 | 0.003831436 |
| | phenylalanylglycine | 0.59 | 0.040228657 | 0.019378354 | 0.57 | 0.034453387 | 0.01596518 |
| | prolylglycine | 1.34 | 0.644428855 | 0.191653754 | 1.86 | 0.388471706 | 0.124454785 |
| | threonylphenylalanine | 0.47 | 0.018130817 | 0.010011583 | 0.45 | 0.013922919 | 0.007480937 |
| | tryptophylglycine | 0.26 | 3.0237E−07 | 9.3243E−07 | 0.25 | 5.129E−08 | 1.6545E−07 |
| | valylglutamine | 0.55 | 0.002175392 | 0.001628628 | 0.53 | 0.001007463 | 0.000770504 |
| | valylglycine | 0.69 | 0.070042126 | 0.030544525 | 0.66 | 0.0298769 | 0.014184087 |
| | valylleucine | 0.17 | 1.3828E−07 | 4.8403E−07 | 0.17 | 2.412E−08 | 8.546E−08 |
| | leucylglutamine* | 0.57 | 0.050650818 | 0.023180913 | 0.55 | 0.052690877 | 0.023089867 |
| Acetylated | phenylacetylglutamate | 6.69 | 0.497392719 | 0.157138227 | 6.53 | 0.521707811 | 0.157588333 |
| Peptides | phenylacetylglutamine | 1.33 | 0.220213832 | 0.081490514 | 1.29 | 0.305541981 | 0.102643345 |
| Glycolysis, | glucose | 1.85 | 0.003627251 | 0.0025479 | 1.91 | 0.003139122 | 0.002084062 |
| Gluconeogenesis, | pyruvate | 7.52 | 3.8E−10 | 4.59E−09 | 10.19 | 1E−11 | 1.6E−10 |
| and Pyruvate | lactate | 7.27 | 2.01E−09 | 1.488E−08 | 11.4 | 4E−11 | 3.6E−10 |
| Metabolism | glycerate | 1.58 | 0.003582419 | 0.002535454 | 1.63 | 0.002702774 | 0.001833064 |
| Pentose | ribose | 3.14 | 3.19343E−06 | 6.3146E−06 | 3.44 | 1.90774E−06 | 3.3568E−06 |
| Metabolism | ribitol | 0.5 | 0.051538943 | 0.023504319 | 0.49 | 0.064759868 | 0.027248744 |
| | ribonate | 0.91 | 0.815789436 | 0.228206283 | 0.92 | 0.88955043 | 0.236353652 |
| | xylose | 2.38 | 0.001394312 | 0.00109117 | 2.64 | 0.000571807 | 0.00047959 |
| | fucose | 7.65 | 2.72058E−06 | 5.5057E−06 | 8.51 | 1.63838E−06 | 2.90858E−06 |
| | arabitol/xylitol | 1.37 | 0.286693758 | 0.101895004 | 1.39 | 0.260052999 | 0.09113373 |
| | ribulose/xylulose | 3.64 | 3.31936E−05 | 4.43213E−05 | 3.99 | 1.45393E−05 | 2.02119E−05 |
| | arabonate/xylonate | 3.11 | 8.7444E−07 | 2.26511E−06 | 3.28 | 5.1438E−07 | 1.14537E−06 |
| | sedoheptulose | 7.82 | 1.2E−10 | 1.87E−09 | 10.32 | 4.18E−12 | 7E−11 |
| Glycogen | maltose | 1.17 | 0.404219577 | 0.135631495 | 1.15 | 0.375295041 | 0.12091036 |
| Metabolism | | | | | | | |
| Disaccharides | lactose | 2.31 | 0.44309181 | 0.143291924 | 2.33 | 0.479282533 | 0.147068435 |
| and | lacto-N-fucopentaose I | 0.01 | 4.97E−08 | 2.0766E−07 | 0.01 | 3.687E−08 | 1.2537E−07 |
| Oligosaccharides | | | | | | | |
| | lacto-N-fucopentaose III | 0.01 | 5.00E−15 | 1.29E−12 | 0.01 | 2.40E−14 | 1.22E−12 |
| | lacto-N-fucopentaose V | 0.02 | 9.46729E−05 | 0.000105706 | 0.02 | 5.89472E−05 | 6.50534E−05 |
| | 3-sialyllactose | 0.03 | 1.91E−09 | 1.452E−08 | 0.02 | 0.000000001 | 5.84E−09 |
| | 6′-sialyllactose | 0.18 | 1.32558E−05 | 2.04389E−05 | 0.18 | 1.32507E−05 | 1.85228E−05 |
| | 2-fucosyllactose | 0.04 | 1.748E−08 | 8.542E−08 | 0.04 | 1.899E−08 | 7.025E−08 |
| | 3-fucosyllactose | 0.01 | 5.80E−14 | 5.55E−12 | 0.01 | 5.60E−14 | 2.02E−12 |
| | Lewis X trisaccharide | 0.11 | 2.354E−08 | 1.1085E−07 | 0.11 | 3.78E−09 | 1.864E−08 |
| | maltitol/lactitol/ | 0.27 | 0.000235301 | 0.000234429 | 0.26 | 0.000278435 | 0.000251108 |
| | cellobiotol/palatinol | | | | | | |
| Fructose, | fructose | 2.91 | 2.95676E−05 | 0.000039891 | 3.12 | 1.61135E−05 | 2.2035E−05 |
| Mannose | mannitol/sorbitol | 9.24 | 8.81E−09 | 4.855E−08 | 9.06 | 2.217E−08 | 7.97E−08 |
| and | mannose | 9.68 | 1.1705E−07 | 4.211E−07 | 13.1 | 1.289E−08 | 5.15E−08 |
| Galactose | galactitol (dulcitol) | 2.31 | 0.002470829 | 0.001839179 | 2.48 | 0.001941622 | 0.001368479 |
| Metabolism | galactonate | 2.95 | 9.0798E−06 | 1.50769E−05 | 3.1 | 7.61458E−06 | 1.14728E−05 |
| Nucleotide | UDP-glucose/UDP- | 1.08 | 0.329876801 | 0.114698043 | 1.08 | 0.329876801 | 0.108927748 |
| Sugar | galactose | | | | | | |
| | UDP-N- | 1 | | | 1 | | |
| | acetylglucosamine | | | | | | |
| | UDP-N- | 1 | | | 1 | | |
| | acetylgalactosamine | | | | | | |
| Aminosugar | glucosamine 6-sulfate | 9.03 | 6.00224E−05 | 7.23162E−05 | 10.4 | 2.64419E−05 | 3.35585E−05 |
| Metabolism | glucuronate | 0.39 | 0.002061669 | 0.00155541 | 0.37 | 0.000389482 | 0.000337933 |
| | N-acetylglucosamine | 15.81 | 1.33759E−06 | 3.03933E−06 | 36.03 | 2.5412E−07 | 6.2686E−07 |
| | 6-sulfate | | | | | | |
| | N-acetylneuraminate | 8.08 | 1.1E−10 | 1.69E−09 | 8.5 | 1.1E−10 | 8.7E−10 |
| | N-acetyl-beta- | 0.96 | 0.147998844 | 0.05870913 | 0.95 | 0.134637979 | 0.051407193 |
| | glucosaminylamine | | | | | | |
| | N-acetylmuramate | 2 | 0.045842572 | 0.021314881 | 1.99 | 0.045592708 | 0.020254711 |
| | 6-sialyl-N- | 0.09 | 1.25745E−05 | 1.96004E−05 | 0.09 | 2.42347E−05 | 3.12713E−05 |
| | acetyllactosamine | | | | | | |
| | N-acetyl- | 22.24 | 1.8265E−07 | 5.9891E−07 | 24 | 1.2721E−07 | 3.4793E−07 |
| | glucosaminylasparagine | | | | | | |
| | erythronate* | 1.72 | 0.000795674 | 0.000682478 | 2.07 | 9.97006E−05 | 0.000100346 |
| | N- | 5.9 | 3.114E−08 | 1.3909E−07 | 8.16 | 2.33E−09 | 1.223E−08 |
| | acetylglucosamine/N- | | | | | | |
| | acetylgalactosamine | | | | | | |
| Advanced | N6- | 1.67 | 0.141847126 | 0.056441697 | 1.65 | 0.142322005 | 0.053850796 |
| Glycation | carboxymethyllysine | | | | | | |
| End- | | | | | | | |
| product | | | | | | | |
| TCA Cycle | citrate | 4.21 | 3.13357E−06 | 6.24392E−06 | 4.91 | 1.43212E−06 | 2.59253E−06 |
| | aconitate [cis or trans] | 6.29 | 2.539E−08 | 1.1746E−07 | 8.38 | 3.4E−09 | 1.729E−08 |
| | alpha-ketoglutarate | 0.83 | 0.920945165 | 0.248239502 | 0.83 | 0.898116638 | 0.238377976 |
| | succinylcarnitine | 2.34 | 0.000130155 | 0.000138744 | 2.34 | 0.000211633 | 0.000196236 |
| | (C4-DC) | | | | | | |
| | succinate | 0.25 | 5.46211E−05 | 6.73755E−05 | 0.25 | 6.37003E−05 | 6.96876E−05 |
| | fumarate | 0.28 | 0.000222271 | 0.000222607 | 0.28 | 0.000396475 | 0.000342819 |
| | malate | 1.1 | 0.148617182 | 0.058864272 | 1.11 | 0.135532177 | 0.051667969 |
| | tricarballylate | 0.02 | 0.157246468 | 0.06162259 | 0.02 | 0.236196236 | 0.084299612 |
| | citraconate/glutaconate | 2.42 | 1.56113E−05 | 2.32408E−05 | 2.52 | 9.92194E−06 | 1.45148E−05 |
| | 2- | 0.73 | 0.052122777 | 0.023687171 | 0.72 | 0.054974609 | 0.023890504 |
| | methylcitrate/homocitrate | | | | | | |
| Oxidative | phosphate | 3.82 | 5.777E−08 | 2.3022E−07 | 4.34 | 8.17E−09 | 3.544E−08 |
| Phosphorylation | | | | | | | |
| Fatty Acid | malonylcarnitine | 1.97 | 1.45149E−06 | 3.26947E−06 | 2.02 | 9.3232E−07 | 1.90723E−06 |
| Synthesis | malonate | 3.75 | 0.002696085 | 0.001951734 | 5.53 | 0.0008433 | 0.000671485 |
| | 2- | 0.12 | 2.5E−10 | 3.66E−09 | 0.12 | 7.6E−10 | 4.66E−09 |
| | methylmalonylcarnitine | | | | | | |
| | (C4-DC) | | | | | | |
| Medium | caprate (10:0) | 0.99 | 0.717189568 | 0.207110457 | 0.99 | 0.768984602 | 0.208727481 |
| Chain Fatty | laurate (12:0) | 1.24 | 0.413892693 | 0.137276827 | 1.28 | 0.339006212 | 0.111357791 |
| Acid | 5-dodecenoate | 1.27 | 0.640967965 | 0.191063203 | 1.35 | 0.535205638 | 0.160421415 |
| | (12:1n7) | | | | | | |
| Long Chain | myristate (14:0) | 1.96 | 0.013220591 | 0.007576575 | 2.12 | 0.00747399 | 0.004353219 |
| Fatty Acid | myristoleate (14:1n5) | 1.38 | 0.317003558 | 0.110966764 | 1.47 | 0.231808007 | 0.08306583 |
| | pentadecanoate (15:0) | 3.1 | 1.47497E−05 | 2.22135E−05 | 3.31 | 6.69585E−06 | 1.02109E−05 |
| | palmitate (16:0) | 2.28 | 5.99834E−06 | 1.04281E−05 | 2.42 | 2.29022E−06 | 3.94698E−06 |
| | palmitoleate (16:1n7) | 1.22 | 0.387561025 | 0.131060503 | 1.28 | 0.302895132 | 0.101890201 |
| | margarate (17:0) | 4.42 | 2.16588E−06 | 4.67535E−06 | 5 | 7.3456E−07 | 1.56634E−06 |
| | stearate (18:0) | 3.02 | 9.5102E−07 | 2.43909E−06 | 3.32 | 2.4349E−07 | 6.1268E−07 |
| | oleate/vaccenate | 1.34 | 0.051838077 | 0.023599191 | 1.41 | 0.030220416 | 0.014320153 |
| | (18:1) | | | | | | |
| | nonadecanoate (19:0) | 5.4 | 2.1146E−07 | 6.7626E−07 | 5.99 | 8.071E−08 | 2.3079E−07 |
| | 10-nonadecenoate | 1.51 | 0.047594393 | 0.021976242 | 1.61 | 0.03127373 | 0.014763666 |
| | (19:1n9) | | | | | | |
| | trans-nonadecenoate | 1.59 | 0.001107784 | 0.000908089 | 2.6 | 5.00588E−05 | 5.68612E−05 |
| | (tr 19:1)* | | | | | | |
| | arachidate (20:0) | 4.69 | 2.48696E−06 | 5.1537E−06 | 5.54 | 6.3743E−07 | 1.38266E−06 |
| | eicosenoate (20:1) | 1.57 | 0.01205826 | 0.007003422 | 1.69 | 0.006990048 | 0.004109397 |
| | behenate (22:0)* | 3.82 | 7.81399E−06 | 1.31435E−05 | 4.37 | 2.40999E−06 | 4.12515E−06 |
| | erucate (22:1n9) | 1.58 | 0.012137278 | 0.007017845 | 1.71 | 0.006775234 | 0.004011226 |
| | nervonate (24:1n9)* | 2.17 | 0.00191334 | 0.001462018 | 2.4 | 0.000915545 | 0.000722156 |
| Polyunsaturated | heneicosapentaenoate | 0.54 | 0.980207543 | 0.260687129 | 0.51 | 0.997474429 | 0.256366221 |
| Fatty | (21:5n3) | | | | | | |
| Acid | hexadecadienoate | 1.09 | 0.586088568 | 0.17881999 | 1.16 | 0.466985973 | 0.144351451 |
| (n3 and n6) | (16:2n6) | | | | | | |
| | hexadecatrienoate | 0.93 | 0.744052967 | 0.214151859 | 1.07 | 0.551867145 | 0.1631724 |
| | (16:3n3) | | | | | | |
| | stearidonate (18:4n3) | 0.94 | 0.679711365 | 0.19866866 | 0.98 | 0.570321504 | 0.16576167 |
| | eicosapentaenoate | 0.55 | 0.863400819 | 0.237170828 | 0.55 | 0.974871855 | 0.253418078 |
| | (EPA; 20:5n3) | | | | | | |
| | docosapentaenoate | 0.99 | 0.303554384 | 0.106977353 | 1.06 | 0.217719267 | 0.078596971 |
| | (n3 DPA; 22:5n3) | | | | | | |
| | docosahexaenoate | 0.96 | 0.47671817 | 0.152265445 | 0.97 | 0.414862222 | 0.13146953 |
| | (DHA; 22:6n3) | | | | | | |
| | docosatrienoate | 1.05 | 0.262905941 | 0.095247781 | 1.15 | 0.16789842 | 0.062126862 |
| | (22:3n3) | | | | | | |
| | nisinate (24:6n3) | 0.53 | 0.430504429 | 0.141338711 | 0.6 | 0.6267328 | 0.178390565 |
| | linoleate (18:2n6) | 1.06 | 0.819231625 | 0.228921973 | 1.13 | 0.66597156 | 0.186975924 |
| | linolenate [alpha or | 1.03 | 0.722112645 | 0.208299928 | 1.12 | 0.578894019 | 0.167618137 |
| | gamma; (18:3n3 or 6)] | | | | | | |
| | dihomo-linolenate | 0.93 | 0.822917851 | 0.229210253 | 0.97 | 0.678120929 | 0.189165674 |
| | (20:3n3 or n6) | | | | | | |
| | arachidonate (20:4n6) | 1.02 | 0.717111489 | 0.207110457 | 1.08 | 0.545107878 | 0.162510615 |
| | adrenate (22:4n6) | 0.89 | 0.698929204 | 0.202968592 | 0.94 | 0.535027771 | 0.160421415 |
| | docosapentaenoate | 0.95 | 0.899304427 | 0.243929242 | 1.01 | 0.733562246 | 0.201137009 |
| | (n6 DPA; 22:5n6) | | | | | | |
| | docosadienoate | 1.65 | 0.019629795 | 0.010571368 | 1.75 | 0.012819211 | 0.006936655 |
| | (22:2n6) | | | | | | |
| | dihomo-linoleate | 1.35 | 0.043791896 | 0.02054707 | 1.45 | 0.025922489 | 0.012665175 |
| | (20:2n6) | | | | | | |
| Fatty Acid | PAHSA (16:0/OH-18:0) | 9.91 | 1.52E−09 | 1.271E−08 | 21.04 | 4E−11 | 3.7E−10 |
| Hydroxyl | LAHSA | 4.48 | 5.7188E−07 | 1.59288E−06 | 6.53 | 6.378E−08 | 1.957E−07 |
| Fatty Acid | (18:2/OH-18:0)* | | | | | | |
| | OAHSA | 5.11 | 1.7406E−07 | 5.8557E−07 | 6.69 | 3.432E−08 | 1.1831E−07 |
| | (18:1/OH-18:0)* | | | | | | |
| Fatty Acid, | 13-methylmyristate | 1.12 | 0.786849472 | 0.222140861 | 1.12 | 0.747882001 | 0.204015508 |
| Branched | 15-methylpalmitate | 2.23 | 0.000186517 | 0.000189469 | 2.36 | 0.000109084 | 0.000108372 |
| | 17-methylstearate | 4.44 | 2.98152E−06 | 5.98699E−06 | 4.94 | 1.14763E−06 | 2.24267E−06 |
| Fatty Acid, | glutarate | 0.68 | 0.027088373 | 0.014005729 | 0.66 | 0.029183798 | 0.013986989 |
| Dicarboxylate | (pentanedioate) | | | | | | |
| | 3-methylglutarate/2- | 0.88 | 0.563051866 | 0.173219514 | 0.87 | 0.549583459 | 0.162879972 |
| | methylglutarate | | | | | | |
| | 2-hydroxyglutarate | 1.47 | 0.036451173 | 0.017815423 | 1.49 | 0.034746141 | 0.016071241 |
| | adipate | 0.57 | 0.393358405 | 0.13267458 | 0.55 | 0.341198801 | 0.111931895 |
| | 2-hydroxyadipate | 0.28 | 0.760917827 | 0.217076303 | 0.27 | 0.73437048 | 0.201137009 |
| | maleate | 2.41 | 1.84918E−05 | 2.676E−05 | 2.9 | 8.6085E−07 | 1.79013E−06 |
| | suberate | 0.82 | 0.277159971 | 0.098890459 | 0.81 | 0.278985257 | 0.096161253 |
| | (octanedioate) | | | | | | |
| | azelate | 0.72 | 0.048127092 | 0.022182669 | 0.71 | 0.051211819 | 0.022511634 |
| | (nonanedioate) | | | | | | |
| | sebacate | 1.08 | 0.623922305 | 0.187492436 | 1.08 | 0.598289651 | 0.17184983 |
| | (decanedioate) | | | | | | |
| | hexadecanedioate | 1.1 | 0.234642057 | 0.086091775 | 1.08 | 0.312674205 | 0.104620294 |
| Fatty Acid, | palmitic amide | 0.9 | 0.655352936 | 0.193348692 | 0.91 | 0.737881577 | 0.201796771 |
| Amide | oleamide | 0.53 | 0.07918835 | 0.034271482 | 0.52 | 0.093609134 | 0.037867755 |
| | linoleamide (18:2n6) | 0.47 | 0.028349033 | 0.014570291 | 0.47 | 0.040569179 | 0.018359597 |
| Fatty Acid, | 1-dihomo- | 0.86 | 0.976149624 | 0.260410006 | 0.98 | 0.748381701 | 0.204015508 |
| Keto | linoleoylglycerol (20:2) | | | | | | |
| Fatty Acid | butyrylcarnitine (C4) | 1.97 | 0.028438991 | 0.014587582 | 1.95 | 0.028157781 | 0.013598857 |
| Metabolism | butyrylglycine | 0.44 | 0.006983928 | 0.004390996 | 0.43 | 0.006542024 | 0.003891468 |
| (also BCAA | propionylcarnitine | 3.72 | 2.74442E−05 | 3.82206E−05 | 3.8 | 2.77239E−05 | 3.43638E−05 |
| Metabolism) | (C3) | | | | | | |
| Fatty Acid | N-palmitoylglycine | 1.55 | 0.68105443 | 0.19866866 | 1.5 | 0.570506089 | 0.16576167 |
| Metabolism | | | | | | | |
| (Acyl Glycine) | | | | | | | |
| Fatty Acid | acetylcarnitine (C2) | 4.2 | 7.63624E−05 | 8.79138E−05 | 4.29 | 7.33413E−05 | 7.75377E−05 |
| Metabolism | 3- | 4.39 | 2.83834E−05 | 3.91573E−05 | 4.47 | 2.37662E−05 | 3.08248E−05 |
| (Acyl | hydroxybutyrylcarnitine | | | | | | |
| Carnitine) | (1) | | | | | | |
| | 3- | 2.59 | 0.000123343 | 0.000132574 | 2.71 | 8.35198E−05 | 8.57758E−05 |
| | hydroxybutyrylcarnitine | | | | | | |
| | (2) | | | | | | |
| | hexanoylcarnitine (C6) | 2.93 | 9.71886E−05 | 0.000108049 | 2.95 | 0.000105544 | 0.000105384 |
| | octanoylcarnitine (C8) | 3.42 | 0.000185668 | 0.00018935 | 3.5 | 0.00019882 | 0.000185284 |
| | decanoylcarnitine | 1.69 | 0.020713901 | 0.011063198 | 1.7 | 0.020275731 | 0.010285764 |
| | (C10) | | | | | | |
| | cis-4- | 2.55 | 0.001244339 | 0.000997923 | 2.6 | 0.001282736 | 0.000952093 |
| | decenoylcarnitine | | | | | | |
| | (C10:1) | | | | | | |
| | laurylcarnitine (C12) | 1.13 | 0.081436288 | 0.035158248 | 1.17 | 0.064906889 | 0.027265012 |
| | myristoylcarnitine | 1.16 | 0.05536783 | 0.02498654 | 1.19 | 0.045918134 | 0.020341249 |
| | (C14) | | | | | | |
| | palmitoylcarnitine | 1.38 | 0.006646945 | 0.004220097 | 1.38 | 0.008098041 | 0.004652084 |
| | (C16) | | | | | | |
| | palmitoleoylcarnitine | 1.13 | 0.101790083 | 0.042391188 | 1.12 | 0.11029616 | 0.043431143 |
| | (C16:1)* | | | | | | |
| | stearoylcarnitine (C18) | 1.16 | 0.210898309 | 0.079059772 | 1.15 | 0.231909985 | 0.08306583 |
| | linoleoylcarnitine | 1.02 | 0.459890307 | 0.147985404 | 1.02 | 0.41574219 | 0.131574044 |
| | (C18:2)* | | | | | | |
| | linolenoylcarnitine | 1.01 | 0.245756389 | 0.089535595 | 1.03 | 0.20937395 | 0.076020572 |
| | (C18:3)* | | | | | | |
| | oleoylcarnitine (C18:1) | 1.17 | 0.149986014 | 0.059135177 | 1.19 | 0.135743236 | 0.051667969 |
| | myristoleoylcarnitine | 1.15 | 0.07368034 | 0.031969644 | 1.16 | 0.065302474 | 0.027339898 |
| | (C14:1)* | | | | | | |
| | suberoylcarnitine | 2.23 | 0.001252923 | 0.001001705 | 2.2 | 0.001806466 | 0.001291305 |
| | (C8-DC) | | | | | | |
| | adipoylcarnitine | 1.92 | 0.002026864 | 0.001538497 | 1.93 | 0.002106293 | 0.001469227 |
| | (C6-DC) | | | | | | |
| | pimeloylcarnitine/3- | 1.15 | 0.423475293 | 0.139917656 | 1.11 | 0.555040489 | 0.163546009 |
| | methyladipoylcarnitine | | | | | | |
| | (C7-DC) | | | | | | |
| | arachidoylcarnitine | 1.1 | 0.517670816 | 0.162539994 | 1.09 | 0.547184499 | 0.162755831 |
| | (C20)* | | | | | | |
| | arachidonoylcarnitine | 0.67 | 0.217135008 | 0.080908955 | 0.64 | 0.118668973 | 0.04615031 |
| | (C20:4) | | | | | | |
| | adrenoylcarnitine | 0.71 | 0.224073763 | 0.082565039 | 0.69 | 0.170257886 | 0.062907416 |
| | (C22:4)* | | | | | | |
| | behenoylcarnitine | 0.95 | 0.874838984 | 0.239550729 | 0.94 | 0.840778588 | 0.225298227 |
| | (C22)* | | | | | | |
| | dihomo- | 1.13 | 0.337838193 | 0.116683108 | 1.11 | 0.371438142 | 0.12040384 |
| | linolenoylcarnitine | | | | | | |
| | (20:3n3 or 6)* | | | | | | |
| | dihomo- | 1 | 0.76987203 | 0.218667485 | 0.99 | 0.778347391 | 0.211041187 |
| | linoleoylcarnitine | | | | | | |
| | (C20:2)* | | | | | | |
| | eicosenoylcarnitine | 0.96 | 0.857550759 | 0.23606452 | 0.95 | 0.911546543 | 0.240925973 |
| | (C20:1)* | | | | | | |
| | erucoylcarnitine | 1.09 | 0.432767579 | 0.141716356 | 1.07 | 0.483495476 | 0.148000206 |
| | (C22:1)* | | | | | | |
| | docosahexaenoylcarnitine | 0.81 | 0.749493374 | 0.215001037 | 0.78 | 0.553889279 | 0.163546009 |
| | (C22:6)* | | | | | | |
| | lignoceroylcarnitine | 1.1 | 0.522067504 | 0.16372203 | 1.09 | 0.531366176 | 0.159738709 |
| | (C24)* | | | | | | |
| | margaroylcarnitine* | 1.52 | 0.003167131 | 0.002266303 | 1.53 | 0.003459182 | 0.002266647 |
| | nervonoylcarnitine | 1.16 | 0.266089669 | 0.09626657 | 1.16 | 0.259416991 | 0.09113373 |
| | (C24:1)* | | | | | | |
| | cerotoylcarnitine | 0.85 | 0.266957252 | 0.096445745 | 0.84 | 0.260490576 | 0.091160112 |
| | (C26)* | | | | | | |
| | ximenoylcarnitine | 1.22 | 0.085760467 | 0.036658528 | 1.23 | 0.07777453 | 0.031976262 |
| | (C26:1)* | | | | | | |
| Carnitine | deoxycarnitine | 0.37 | 0.009078632 | 0.005494614 | 0.35 | 0.008615447 | 0.004875734 |
| Metabolism | carnitine | 1.35 | 0.016274047 | 0.009144398 | 1.45 | 0.009619126 | 0.005354746 |
| Ketone | 3-hydroxybutyrate | 0.45 | 0.000279805 | 0.000273508 | 0.44 | 0.000297163 | 0.000265147 |
| Bodies | (BHBA) | | | | | | |
| Fatty Acid | palmitoylcholine | 0.91 | 0.648870773 | 0.192521066 | 0.93 | 0.561209844 | 0.164367594 |
| Metabolism | oleoylcholine | 0.68 | 0.549445129 | 0.170246643 | 0.74 | 0.393412607 | 0.125621443 |
| (Acyl | palmitoloelycholine | 5.61 | 2.82E−09 | 2.026E−08 | 7.44 | 3.5E−10 | 2.37E−09 |
| Choline) | linoleoylcholine* | 0.54 | 0.963756612 | 0.257900699 | 0.57 | 0.832507127 | 0.223557936 |
| Fatty Acid, | 2-hydroxydecanoate | 0.74 | 0.747379416 | 0.214870526 | 0.75 | 0.868015122 | 0.231119828 |
| Monohydroxy | 2-hydroxypalmitate | 0.96 | 0.304410982 | 0.1069925 | 0.94 | 0.349241452 | 0.114272352 |
| | 2-hydroxystearate | 1.92 | 0.002031245 | 0.001538497 | 1.99 | 0.001320215 | 0.000975055 |
| | 2-hydroxybehenate | 2.8 | 0.004566977 | 0.003062108 | 2.75 | 0.005103371 | 0.003147304 |
| | 3-hydroxysebacate | 1.46 | 0.029276531 | 0.014899179 | 1.46 | 0.030705721 | 0.014522768 |
| | 3-hydroxylaurate | 0.67 | 0.036532614 | 0.017821601 | 0.68 | 0.053196372 | 0.02319786 |
| | 3-hydroxypalmitate | 1.25 | 0.874579951 | 0.239550729 | 1.26 | 0.801037561 | 0.216261241 |
| | 13-HODE + 9-HODE | 0.64 | 0.000951753 | 0.00079786 | 0.66 | 0.001321548 | 0.000975055 |
| | 10-hydroxystearate | 2.41 | 0.000159777 | 0.000166888 | 2.75 | 7.40765E−05 | 7.79874E−05 |
| Fatty Acid, | 12,13-DiHOME | 1.43 | 0.048464344 | 0.022258902 | 1.38 | 0.078191774 | 0.032095365 |
| Dihydroxy | 9,10-DiHOME | 1.8 | 0.116898291 | 0.047536723 | 1.74 | 0.15573012 | 0.058310272 |
| Endocannabinoid | oleoyl ethanolamide | 1.05 | 0.498040317 | 0.157138227 | 1.09 | 0.388769711 | 0.124454785 |
| | palmitoyl | 1.31 | 0.057881556 | 0.025940173 | 1.38 | 0.03262758 | 0.01523133 |
| | ethanolamide | | | | | | |
| | stearoyl ethanolamide | 1.47 | 0.011354022 | 0.006654079 | 1.57 | 0.004570671 | 0.002889606 |
| | linoleoyl | 0.83 | 0.978318978 | 0.260470733 | 0.86 | 0.819903703 | 0.220408694 |
| | ethanolamide | | | | | | |
| | arachidoyl | 1.76 | 0.097583907 | 0.040836457 | 1.92 | 0.05007203 | 0.022064842 |
| | ethanolamide (20:0)* | | | | | | |
| | palmitoleoyl | 1.27 | 0.058765533 | 0.026200348 | 1.31 | 0.039734683 | 0.018079497 |
| | ethanolamide* | | | | | | |
| | margaroyl | 1.23 | 0.384974271 | 0.130526548 | 1.2 | 0.456526846 | 0.141640415 |
| | ethanolamide* | | | | | | |
| | N-palmitoylserine | 0.9 | 0.104409999 | 0.043342904 | 0.86 | 0.092134441 | 0.037331215 |
| Inositol | myo-inositol | 4.51 | 7.0419E−06 | 1.19223E−05 | 5.17 | 4.0355E−06 | 0.000006551 |
| Metabolism | chiro-inositol | 0.91 | 0.773497746 | 0.219456668 | 0.87 | 0.572420048 | 0.166035731 |
| Phospholipid | choline | 1.03 | 0.844880368 | 0.233569502 | 1.03 | 0.901310199 | 0.238973528 |
| Metabolism | choline phosphate | 0.78 | 0.762895906 | 0.217162283 | 0.91 | 0.970767263 | 0.252859758 |
| | glycerophosphorylcholine | 7.47 | 9.8298E−07 | 2.45895E−06 | 9.9 | 1.8424E−07 | 4.7304E−07 |
| | (GPC) | | | | | | |
| | ethanolamine | 2.36 | 0.009955306 | 0.005983259 | 2.5 | 0.005621649 | 0.003441626 |
| | glycerophosphoethanolamine | 1.08 | 0.441847679 | 0.143068195 | 1.05 | 0.476950104 | 0.146531424 |
| | glycerophosphoserine * | 2.45 | 0.003825086 | 0.002642229 | 2.49 | 0.003281888 | 0.002167407 |
| | glycerophosphoinositol* | 3.2 | 0.000459144 | 0.00042629 | 4.38 | 7.97862E−05 | 8.26158E−05 |
| | trimethylamine N- | 0.97 | 0.572197046 | 0.175824157 | 0.93 | 0.706278524 | 0.195288348 |
| | oxide | | | | | | |
| Phosphatidylcholine | 1,2-dipalmitoyl-GPC | 0.62 | 0.032032909 | 0.015926457 | 0.6 | 0.027667212 | 0.013439322 |
| (PC) | (16:0/16:0) | | | | | | |
| | 1-palmitoyl-2- | 0.56 | 0.055267583 | 0.02498654 | 0.55 | 0.05748792 | 0.024896723 |
| | palmitoleoyl-GPC | | | | | | |
| | (16:0/16:1)* | | | | | | |
| | 1-palmitoyl-2- | 0.68 | 0.089635455 | 0.037939265 | 0.66 | 0.076825216 | 0.031670228 |
| | stearoyl-GPC | | | | | | |
| | (16:0/18:0) | | | | | | |
| | 1-palmitoyl-2-oleoyl- | 0.62 | 0.131382834 | 0.052519929 | 0.6 | 0.13404694 | 0.051259307 |
| | GPC (16:0/18:1) | | | | | | |
| | 1-palmitoyl-2- | 0.44 | 0.043864635 | 0.02054707 | 0.43 | 0.041546626 | 0.018734551 |
| | linoleoyl-GPC | | | | | | |
| | (16:0/18:2) | | | | | | |
| | 1-palmitoyl-2-alpha- | 1.31 | 0.48525496 | 0.154420902 | 1.36 | 0.402371167 | 0.127994731 |
| | linolenoyl-GPC | | | | | | |
| | (16:0/18:3n3)* | | | | | | |
| | 1-palmitoyl-2-gamma- | 0.94 | 0.749404221 | 0.215001037 | 0.92 | 0.761803701 | 0.207225439 |
| | linolenoyl-GPC | | | | | | |
| | (16:0/18:3n6)* | | | | | | |
| | 1-palmitoyl-2- | 0.72 | 0.196434606 | 0.074500262 | 0.7 | 0.211605739 | 0.076720194 |
| | arachidonoyl-GPC | | | | | | |
| | (16:0/20:4n6) | | | | | | |
| | 1-stearoyl-2-oleoyl- | 0.74 | 0.250765591 | 0.091232265 | 0.73 | 0.266785509 | 0.092846527 |
| | GPC (18:0/18:1) | | | | | | |
| | 1-oleoyl-2-linoleoyl- | 0.75 | 0.099810235 | 0.041633602 | 0.73 | 0.098963751 | 0.039645386 |
| | GPC (18:1/18:2)* | | | | | | |
| | 1,2-dilinoleoyl-GPC | 2.25 | 0.029098976 | 0.01483797 | 2.36 | 0.019986922 | 0.010180303 |
| | (18:2/18:2) | | | | | | |
| | 1-stearoyl-2- | 0.97 | 0.594453076 | 0.18073343 | 0.97 | 0.64580907 | 0.182786493 |
| | arachidonoyl-GPC | | | | | | |
| | (18:0/20:4) | | | | | | |
| Phosphatidylethanolamine | 1,2-dipalmitoyl-GPE | 0.57 | 0.001404854 | 0.001096108 | 0.56 | 0.000896984 | 0.00070974 |
| (PE) | (16:0/16:0)* | | | | | | |
| | 1-palmitoyl-2- | 0.11 | 2.43891E−06 | 5.09488E−06 | 0.11 | 1.38557E−06 | 2.54479E−06 |
| | linoleoyl-GPE | | | | | | |
| | (16:0/18:2) | | | | | | |
| Lysophospholipid | 1-palmitoyl-GPC (16:0) | 0.48 | 0.02525492 | 0.013216039 | 0.47 | 0.027311903 | 0.013292392 |
| | 2-palmitoyl-GPC | 0.39 | 0.063420077 | 0.027938941 | 0.38 | 0.072629199 | 0.030068418 |
| | (16:0)* | | | | | | |
| | 1-palmitoleoyl-GPC | 0.23 | 0.003629512 | 0.0025479 | 0.22 | 0.003156809 | 0.002090289 |
| | (16:1)* | | | | | | |
| | 1-stearoyl-GPC (18:0) | 0.73 | 0.203616244 | 0.076886273 | 0.72 | 0.221444094 | 0.079806407 |
| | 1-oleoyl-GPC (18:1) | 0.49 | 0.092135824 | 0.038870548 | 0.48 | 0.095417245 | 0.038413916 |
| | 1-linoleoyl-GPC (18:2) | 0.36 | 0.008306119 | 0.005110655 | 0.34 | 0.007665173 | 0.004443999 |
| | 1-linolenoyl-GPC | 0.58 | 0.031817987 | 0.015881984 | 0.56 | 0.022643932 | 0.011283519 |
| | (18:3)* | | | | | | |
| | 1-palmitoyl-GPE (16:0) | 0.13 | 4.571E−08 | 1.9411E−07 | 0.13 | 5.528E−08 | 1.7386E−07 |
| | 1-stearoyl-GPE (18:0) | 0.91 | 0.493731407 | 0.156541397 | 0.92 | 0.548404671 | 0.162795477 |
| | 2-stearoyl-GPE (18:0)* | 1.07 | 0.612251815 | 0.184413256 | 1.05 | 0.623844769 | 0.178173188 |
| | 1-oleoyl-GPE (18:1) | 0.3 | 5.22787E−05 | 6.51061E−05 | 0.29 | 5.19255E−05 | 5.85892E−05 |
| | 1-palmitoyl-GPS | 0.24 | 5.1417E−07 | 1.44769E−06 | 0.23 | 3.6791E−07 | 8.7333E−07 |
| | (16:0)* | | | | | | |
| | 1-stearoyl-GPS (18:0)* | 0.47 | 0.000111396 | 0.000120734 | 0.46 | 7.15573E−05 | 7.62929E−05 |
| | 1-oleoyl-GPS (18:1) | 0.26 | 4.154E−07 | 1.23684E−06 | 0.25 | 2.7105E−07 | 6.4954E−07 |
| | 1-palmitoyl-GPG | 0.19 | 0.001206409 | 0.000976574 | 0.18 | 0.000193802 | 0.000181279 |
| | (16:0)* | | | | | | |
| | 1-stearoyl-GPG (18:0) | 0.51 | 0.140901248 | 0.056151581 | 0.49 | 0.138398178 | 0.052444991 |
| | 1-oleoyl-GPG (18:1)* | 0.49 | 0.007678001 | 0.004792483 | 0.5 | 0.013106438 | 0.007076858 |
| | 1-linoleoyl-GPG | 0.15 | 0.000824948 | 0.000705253 | 0.15 | 0.000976241 | 0.000751837 |
| | (18:2)* | | | | | | |
| | 1-palmitoyl-GPI (16:0) | 0.74 | 0.753218076 | 0.215187161 | 0.7 | 0.539855633 | 0.161327078 |
| | 1-stearoyl-GPI (18:0) | 1.16 | 0.407212494 | 0.135931428 | 1.16 | 0.385428134 | 0.123699821 |
| | 1-oleoyl-GPI (18:1)* | 0.35 | 0.005164646 | 0.003412833 | 0.34 | 0.004372476 | 0.00279237 |
| Glycolipid | galactosylglycerol* | 4.74 | 0.033604022 | 0.016519147 | 4.97 | 0.025614801 | 0.012539193 |
| Metabolism | 1-palmitoyl-2- | 1.75 | 0.010631346 | 0.006287452 | 1.8 | 0.008424108 | 0.004806479 |
| | linoleoyl- | | | | | | |
| | digalactosylglycerol | | | | | | |
| | (16:0/18:2)* | | | | | | |
| | 1-palmitoyl-2- | 1.8 | 0.02620073 | 0.013573877 | 1.79 | 0.023888186 | 0.011808837 |
| | linoleoyl- | | | | | | |
| | galactosylglycerol | | | | | | |
| | (16:0/18:2)* | | | | | | |
| Plasmalogen | 1-(1-enyl-palmitoyl)-2- | 0.12 | 1.39363E−05 | 2.12353E−05 | 0.12 | 6.98945E−06 | 1.05944E−05 |
| | oleoyl-GPE (P- | | | | | | |
| | 16:0/18:1)* | | | | | | |
| | 1-(1-enyl-palmitoyl)-2- | 0.57 | 0.00559606 | 0.00365134 | 0.57 | 0.0063154 | 0.003798861 |
| | palmitoyl-GPC (P- | | | | | | |
| | 16:0/16:0)* | | | | | | |
| | 1-(1-enyl-palmitoyl)-2- | 0.75 | 0.303954904 | 0.106977353 | 0.75 | 0.334080655 | 0.10988328 |
| | oleoyl-GPC (P- | | | | | | |
| | 16:0/18:1)* | | | | | | |
| | 1-(1-enyl-palmitoyl)-2- | 0.92 | 0.607583044 | 0.183862172 | 0.91 | 0.625278757 | 0.178380268 |
| | arachidonoyl-GPC (P- | | | | | | |
| | 16:0/20:4)* | | | | | | |
| Lysoplasmalogen | 1-(1-enyl-palmitoyl)- | 0.51 | 0.056104654 | 0.025275022 | 0.49 | 0.054864384 | 0.023883853 |
| | GPC (P-16:0)* | | | | | | |
| | 1-(1-enyl-palmitoyl)- | 0.35 | 0.006092395 | 0.003955266 | 0.33 | 0.004257559 | 0.002761027 |
| | GPE (P-16:0)* | | | | | | |
| | 1-(1-enyl-oleoyl)-GPE | 0.49 | 0.040265843 | 0.019378354 | 0.48 | 0.031728387 | 0.014922306 |
| | (P-18:1)* | | | | | | |
| | 1-(1-enyl-stearoyl)- | 0.6 | 0.148872257 | 0.058875279 | 0.58 | 0.123273066 | 0.047573196 |
| | GPE (P-18:0)* | | | | | | |
| Glycerolipid | glycerol | 4.7 | 9.9673E−07 | 2.45895E−06 | 6.43 | 1.0558E−07 | 2.9517E−07 |
| Metabolism | glycerol 3-phosphate | 5.01 | 0.000845942 | 0.000720718 | 5.21 | 0.000576077 | 0.000481566 |
| | glycerophosphoglycerol | 1.71 | 0.041558479 | 0.019811045 | 1.8 | 0.028536111 | 0.013755171 |
| Monoacylglycerol | 1-myristoylglycerol | 0.56 | 0.554747533 | 0.171188673 | 0.59 | 0.423343765 | 0.133317791 |
| | (14:0) | | | | | | |
| | 1-myristoleoylglycerol | 0.7 | 0.664385417 | 0.195124594 | 0.77 | 0.486925731 | 0.148869117 |
| | (14:1) | | | | | | |
| | 1- | 0.85 | 0.44117202 | 0.143068195 | 0.92 | 0.322067926 | 0.10691041 |
| | pentadecanoylglycerol | | | | | | |
| | (15:0) | | | | | | |
| | 1-palmitoylglycerol | 0.93 | 0.475972969 | 0.152265445 | 1.01 | 0.359139235 | 0.116902845 |
| | (16:0) | | | | | | |
| | 1-palmitoleoylglycerol | 0.71 | 0.64968963 | 0.192521066 | 0.76 | 0.510035314 | 0.154247681 |
| | (16:1)* | | | | | | |
| | 1-margaroylglycerol | 1.31 | 0.399860882 | 0.134343002 | 1.4 | 0.298403355 | 0.100928977 |
| | (17:0) | | | | | | |
| | 1-oleoylglycerol (18:1) | 0.98 | 0.095086154 | 0.039984998 | 1.09 | 0.060446662 | 0.025999092 |
| | 1-linoleoylglycerol | 0.83 | 0.433295972 | 0.141716356 | 0.91 | 0.314841866 | 0.105066159 |
| | (18:2) | | | | | | |
| | 1-linolenoylglycerol | 0.94 | 0.440931248 | 0.143068195 | 1.04 | 0.315976023 | 0.105252537 |
| | (18:3) | | | | | | |
| | 1-dihomo- | 0.77 | 0.597123398 | 0.181332465 | 0.84 | 0.447746392 | 0.139259649 |
| | linolenylglycerol (20:3) | | | | | | |
| | 1-arachidonylglycerol | 0.74 | 0.890411641 | 0.242024 | 0.84 | 0.696155718 | 0.192913344 |
| | (20:4) | | | | | | |
| | 1- | 0.81 | 0.63378675 | 0.189306548 | 0.83 | 0.753415412 | 0.205165461 |
| | eicosapentaenoylglycerol | | | | | | |
| | (20:5)* | | | | | | |
| | 1- | 0.7 | 0.36828915 | 0.125361672 | 0.72 | 0.283271914 | 0.096974579 |
| | docosahexaenoylglycerol | | | | | | |
| | (22:6) | | | | | | |
| | 2-myristoylglycerol | 0.7 | 0.661753942 | 0.194572355 | 0.73 | 0.535549857 | 0.160421415 |
| | (14:0) | | | | | | |
| | 2-palmitoylglycerol | 1.07 | 0.210346403 | 0.078967158 | 1.13 | 0.150590405 | 0.056554116 |
| | (16:0) | | | | | | |
| | 2-palmitoleoylglycerol | 0.73 | 0.878576239 | 0.24002799 | 0.75 | 0.987356565 | 0.255134788 |
| | (16:1)* | | | | | | |
| | 2-oleoylglycerol (18:1) | 1.17 | 0.405469653 | 0.135699392 | 1.28 | 0.302146562 | 0.101774453 |
| | 2-linoleoylglycerol | 0.85 | 0.824514205 | 0.229408216 | 0.91 | 0.676602074 | 0.188951462 |
| | (18:2) | | | | | | |
| | 2-arachidonoylglycerol | 0.69 | 0.900412093 | 0.243974217 | 0.77 | 0.688917589 | 0.191888624 |
| | (20:4) | | | | | | |
| | 2- | 0.89 | 0.78651896 | 0.222140861 | 0.9 | 0.689407394 | 0.191888624 |
| | docosahexaenoylglycerol | | | | | | |
| | (22:6)* | | | | | | |
| Diacylglycerol | diacylglycerol | 1.22 | 0.350223948 | 0.120319218 | 1.39 | 0.206517604 | 0.075091833 |
| | (12:0/18:1, 14:0/16:1, | | | | | | |
| | 16:0/14:1) [1]* | | | | | | |
| | diacylglycerol | 2 | 0.021458873 | 0.011414015 | 2.49 | 0.009313201 | 0.005207487 |
| | (12:0/18:1, 14:0/16:1, | | | | | | |
| | 16:0/14:1) [2]* | | | | | | |
| | diacylglycerol | 1.25 | 0.217159133 | 0.080908955 | 1.53 | 0.124375682 | 0.047925209 |
| | (14:0/18:1, 16:0/16:1) | | | | | | |
| | [1]* | | | | | | |
| | diacylglycerol | 1.79 | 0.004833004 | 0.003210059 | 2.2 | 0.001768349 | 0.001267659 |
| | (14:0/18:1, 16:0/16:1) | | | | | | |
| | [2]* | | | | | | |
| | diacylglycerol | 1.66 | 0.038712576 | 0.01874385 | 2.08 | 0.018228333 | 0.009405486 |
| | (16:1/18:2 [2], | | | | | | |
| | 16:0/18:3 [1])* | | | | | | |
| | palmitoyl-myristoyl- | 2.62 | 0.008734368 | 0.005336127 | 3.41 | 0.002888333 | 0.001928516 |
| | glycerol (16:0/14:0) | | | | | | |
| | [2] | | | | | | |
| | myristoyl-linoleoyl- | 1.45 | 0.033379299 | 0.016500854 | 1.66 | 0.016349717 | 0.008517357 |
| | glycerol (14:0/18:2) | | | | | | |
| | [1]* | | | | | | |
| | myristoyl-linoleoyl- | 1.4 | 0.045702312 | 0.021292364 | 1.62 | 0.023649393 | 0.011713806 |
| | glycerol (14:0/18:2) | | | | | | |
| | [2]* | | | | | | |
| | palmitoyl-palmitoyl- | 2.56 | 0.040807997 | 0.019575462 | 4.05 | 0.012513985 | 0.006786086 |
| | glycerol (16:0/16:0) | | | | | | |
| | [1]* | | | | | | |
| | palmitoyl-palmitoyl- | 3.56 | 0.000294214 | 0.000284374 | 5.22 | 6.19289E−05 | 6.80455E−05 |
| | glycerol (16:0/16:0) | | | | | | |
| | [2]* | | | | | | |
| | palmitoyl-oleoyl- | 2.47 | 0.003664271 | 0.002558434 | 3.26 | 0.001337736 | 0.000981337 |
| | glycerol (16:0/18:1) | | | | | | |
| | [1]* | | | | | | |
| | palmitoyl-oleoyl- | 2.63 | 0.001240743 | 0.000997923 | 3.37 | 0.000434811 | 0.000374679 |
| | glycerol (16:0/18:1) | | | | | | |
| | [2]* | | | | | | |
| | palmitoyl-linoleoyl- | 1.98 | 0.007711962 | 0.00480211 | 2.36 | 0.003655157 | 0.002382651 |
| | glycerol (16:0/18:2) | | | | | | |
| | [1]* | | | | | | |
| | palmitoyl-linoleoyl- | 2.01 | 0.010138706 | 0.006050981 | 2.44 | 0.004866685 | 0.003038575 |
| | glycerol (16:0/18:2) | | | | | | |
| | [2]* | | | | | | |
| | palmitoyl-linolenoyl- | 1.77 | 0.015977609 | 0.009036637 | 2.59 | 0.004687325 | 0.002948538 |
| | glycerol (16:0/18:3) | | | | | | |
| | [2]* | | | | | | |
| | palmitoleoyl-linoleoyl- | 2.09 | 0.037433538 | 0.018158506 | 2.38 | 0.020332961 | 0.010294042 |
| | glycerol (16:1/18:2) | | | | | | |
| | [1]* | | | | | | |
| | palmitoyl-dihomo- | 1.9 | 0.025926434 | 0.013478864 | 2.72 | 0.010530606 | 0.005836322 |
| | linolenoyl-glycerol | | | | | | |
| | (16:0/20:3n3 or 6) [2]* | | | | | | |
| | palmitoyl- | 0.69 | 0.660951001 | 0.194557106 | 0.7 | 0.548651364 | 0.162795477 |
| | arachidonoyl-glycerol | | | | | | |
| | (16:0/20:4) [1]* | | | | | | |
| | palmitoyl- | 0.99 | 0.577397475 | 0.176785257 | 1.04 | 0.464482505 | 0.143754199 |
| | arachidonoyl-glycerol | | | | | | |
| | (16:0/20:4) [2]* | | | | | | |
| | palmitoyl- | 1.64 | 0.008812664 | 0.005371293 | 1.73 | 0.005200588 | 0.003199417 |
| | docosahexaenoyl- | | | | | | |
| | glycerol (16:0/22:6) | | | | | | |
| | [1]* | | | | | | |
| | palmitoyl- | 1.84 | 0.010704471 | 0.006316278 | 1.75 | 0.01495605 | 0.00795605 |
| | docosahexaenoyl- | | | | | | |
| | glycerol (16:0/22:6) | | | | | | |
| | [2]* | | | | | | |
| | stearoyl-linoleoyl- | 3.38 | 0.000287075 | 0.000279559 | 5.1 | 6.53173E−05 | 7.08405E−05 |
| | glycerol (18:0/18:2) | | | | | | |
| | [2]* | | | | | | |
| | oleoyl-oleoyl-glycerol | 2.63 | 0.005939018 | 0.003865379 | 3.73 | 0.001845997 | 0.001312107 |
| | (18:1/18:1) [1]* | | | | | | |
| | oleoyl-oleoyl-glycerol | 2.34 | 0.012382948 | 0.007128071 | 2.73 | 0.006300823 | 0.003798861 |
| | (18:1/18:1) [2]* | | | | | | |
| | oleoyl-linoleoyl- | 2.17 | 0.018720038 | 0.010251941 | 2.71 | 0.008955518 | 0.005018641 |
| | glycerol (18:1/18:2) | | | | | | |
| | [1] | | | | | | |
| | oleoyl-linoleoyl- | 2.1 | 0.031752695 | 0.015881984 | 2.57 | 0.01619368 | 0.008471147 |
| | glycerol (18:1/18:2) | | | | | | |
| | [2] | | | | | | |
| | oleoyl-linolenoyl- | 2.34 | 0.010157897 | 0.006050981 | 3.26 | 0.003076609 | 0.002047963 |
| | glycerol (18:1/18:3) | | | | | | |
| | [2]* | | | | | | |
| | linoleoyl-linoleoyl- | 2.25 | 0.046581315 | 0.021585417 | 2.67 | 0.02399046 | 0.011836141 |
| | glycerol (18:2/18:2) | | | | | | |
| | [1]* | | | | | | |
| | linoleoyl-linoleoyl- | 2.04 | 0.039568599 | 0.019122576 | 2.49 | 0.018732791 | 0.009599812 |
| | glycerol (18:2/18:2) | | | | | | |
| | [2]* | | | | | | |
| | linoleoyl-linolenoyl- | 1.75 | 0.061972881 | 0.027441404 | 2.2 | 0.029706492 | 0.014137138 |
| | glycerol (18:2/18:3) | | | | | | |
| | [1]* | | | | | | |
| | linoleoyl-linolenoyl- | 2.41 | 0.071075513 | 0.03094308 | 2.8 | 0.040196156 | 0.018223562 |
| | glycerol (18:2/18:3) | | | | | | |
| | [2]* | | | | | | |
| | linolenoyl-linolenoyl- | 1.09 | 0.751273236 | 0.215035342 | 1.33 | 0.443870707 | 0.138568069 |
| | glycerol (18:3/18:3) | | | | | | |
| | [1]* | | | | | | |
| | linolenoyl-linolenoyl- | 1.57 | 0.205588216 | 0.077405227 | 2.29 | 0.091887065 | 0.037291033 |
| | glycerol (18:3/18:3) | | | | | | |
| | [2]* | | | | | | |
| | stearoyl-arachidonoyl- | 0.69 | 0.326514536 | 0.113834581 | 0.72 | 0.439909131 | 0.137501938 |
| | glycerol (18:0/20:4) | | | | | | |
| | [1]* | | | | | | |
| | stearoyl-arachidonoyl- | 1.46 | 0.481374441 | 0.153563324 | 1.65 | 0.353486632 | 0.115361353 |
| | glycerol (18:0/20:4) | | | | | | |
| | [2]* | | | | | | |
| | oleoyl-arachidonoyl- | 1 | 0.439862389 | 0.143068195 | 1.08 | 0.32397688 | 0.107402395 |
| | glycerol (18:1/20:4) | | | | | | |
| | [1]* | | | | | | |
| | oleoyl-arachidonoyl- | 1.21 | 0.311583695 | 0.109365128 | 1.32 | 0.22826641 | 0.082051343 |
| | glycerol (18:1/20:4) | | | | | | |
| | [2]* | | | | | | |
| | linoleoyl- | 1.53 | 0.202507915 | 0.076579396 | 1.72 | 0.130454338 | 0.050037593 |
| | arachidonoyl-glycerol | | | | | | |
| | (18:2/20:4) [1]* | | | | | | |
| | linoleoyl- | 0.97 | 0.941013112 | 0.252597395 | 1.05 | 0.794459558 | 0.214946594 |
| | arachidonoyl-glycerol | | | | | | |
| | (18:2/20:4) [2]* | | | | | | |
| | linoleoyl- | 2.04 | 0.025935581 | 0.013478864 | 2.27 | 0.015172613 | 0.00804159 |
| | docosahexaenoyl- | | | | | | |
| | glycerol (18:2/22:6) | | | | | | |
| | [1]* | | | | | | |
| | linoleoyl- | 1.55 | 0.241711948 | 0.088435242 | 1.63 | 0.180089882 | 0.06624833 |
| | docosahexaenoyl- | | | | | | |
| | glycerol (18:2/22:6) | | | | | | |
| | [2]* | | | | | | |
| Sphingolipid | sphinganine | 0.45 | 0.169883594 | 0.065680605 | 0.44 | 0.198959401 | 0.07265859 |
| Metabolism | 3-ketosphinganine | 0.11 | 0.189406358 | 0.07215163 | 0.11 | 0.160970635 | 0.060093636 |
| | N-palmitoyl- | 1.49 | 0.015362725 | 0.008726979 | 1.59 | 0.006498892 | 0.003874972 |
| | sphinganine | | | | | | |
| | (d18:0/16:0) | | | | | | |
| | N-behenoyl- | 1.16 | 0.15804297 | 0.06184117 | 1.24 | 0.098394315 | 0.039486095 |
| | sphingadienine | | | | | | |
| | (d18:2/22:0)* | | | | | | |
| | myristoyl | 1.33 | 0.230409756 | 0.084698736 | 1.45 | 0.143144688 | 0.054080753 |
| | dihydrosphingomyelin | | | | | | |
| | (d18:0/14:0)* | | | | | | |
| | palmitoyl | 2.23 | 6.23228E−05 | 7.43956E−05 | 2.45 | 1.87581E−05 | 2.524E−05 |
| | dihydrosphingomyelin | | | | | | |
| | (d18:0/16:0)* | | | | | | |
| | behenoyl | 1.25 | 0.342254191 | 0.117893929 | 1.24 | 0.332721851 | 0.109579594 |
| | dihydrosphingomyelin | | | | | | |
| | (d18:0/22:0)* | | | | | | |
| | palmitoyl | 1.18 | 0.321316466 | 0.112324703 | 1.2 | 0.280832161 | 0.09659274 |
| | sphingomyelin | | | | | | |
| | (d18:1/16:0) | | | | | | |
| | stearoyl | 1.24 | 0.220811435 | 0.081595094 | 1.25 | 0.190791247 | 0.069980341 |
| | sphingomyelin | | | | | | |
| | (d18:1/18:0) | | | | | | |
| | behenoyl | 1.31 | 0.268517152 | 0.096693681 | 1.32 | 0.245818368 | 0.087115956 |
| | sphingomyelin | | | | | | |
| | (d18:1/22:0)* | | | | | | |
| | tricosanoyl | 1.27 | 0.242639451 | 0.088524518 | 1.26 | 0.243465611 | 0.086403854 |
| | sphingomyelin | | | | | | |
| | (d18:1/23:0)* | | | | | | |
| | lignoceroyl | 1.17 | 0.555217543 | 0.171188673 | 1.18 | 0.50835252 | 0.153923765 |
| | sphingomyelin | | | | | | |
| | (d18:1/24:0) | | | | | | |
| | sphingomyelin | 1.18 | 0.354875162 | 0.121594607 | 1.22 | 0.28497345 | 0.097292337 |
| | (d18:1/14:0, | | | | | | |
| | d16:1/16:0)* | | | | | | |
| | sphingomyelin | 11.93 | 8E−10 | 7.37E−09 | 20.63 | 2E−11 | 2E−10 |
| | (d17:1/16:0, | | | | | | |
| | d18:1/15:0, | | | | | | |
| | d16:1/17:0)* | | | | | | |
| | sphingomyelin | 0.97 | 0.995932709 | 0.263516498 | 0.97 | 0.953419768 | 0.249633282 |
| | (d18:2/16:0, | | | | | | |
| | d18:1/16:1)* | | | | | | |
| | sphingomyelin | 4.53 | 6.182E−08 | 2.3899E−07 | 5.2 | 1.467E−08 | 5.679E−08 |
| | (d18:1/17:0, | | | | | | |
| | d17:1/18:0, | | | | | | |
| | d19:1/16:0) | | | | | | |
| | sphingomyelin | 0.5 | 0.851561693 | 0.234850249 | 0.48 | 0.867749205 | 0.231119828 |
| | (d18:1/18:1, | | | | | | |
| | d18:2/18:0) | | | | | | |
| | sphingomyelin | 1.27 | 0.275973694 | 0.098603013 | 1.28 | 0.249523998 | 0.088304827 |
| | (d18:1/20:0, | | | | | | |
| | d16:1/22:0)* | | | | | | |
| | sphingomyelin | 1.29 | 0.539231353 | 0.168289705 | 1.32 | 0.463679002 | 0.143682251 |
| | (d18:1/20:1, | | | | | | |
| | d18:2/20:0)* | | | | | | |
| | sphingomyelin | 1.38 | 0.161673744 | 0.062881917 | 1.38 | 0.155111815 | 0.058165315 |
| | (d18:1/21:0, | | | | | | |
| | d17:1/22:0, | | | | | | |
| | d16:1/23:0)* | | | | | | |
| | sphingomyelin | 1.29 | 0.207442722 | 0.077990102 | 1.32 | 0.167025551 | 0.06198619 |
| | (d18:1/22:1, | | | | | | |
| | d18:2/22:0, | | | | | | |
| | d16:1/24:1)* | | | | | | |
| | sphingomyelin | 1.11 | 0.627443399 | 0.187967273 | 1.13 | 0.55155922 | 0.1631724 |
| | (d18:1/24:1, | | | | | | |
| | d18:2/24:0)* | | | | | | |
| | sphingomyelin | 0.87 | 0.988573298 | 0.262104706 | 0.87 | 0.935152665 | 0.245896056 |
| | (d18:2/24:1, | | | | | | |
| | d18:1/24:2)* | | | | | | |
| | sphingosine | 0.78 | 0.155296923 | 0.060950801 | 0.79 | 0.213698698 | 0.077367541 |
| | N-acetylsphingosine | 0.08 | 0.021826737 | 0.0115622 | 0.07 | 0.028122191 | 0.013598857 |
| | phytosphingosine | 0.88 | 0.094484445 | 0.039796576 | 0.89 | 0.115977956 | 0.045243632 |
| | sphingomyelin | 1.11 | 0.893798569 | 0.242689947 | 1.14 | 0.79671927 | 0.215326441 |
| | (d18:0/20:0, | | | | | | |
| | d16:0/22:0)* | | | | | | |
| | sphingomyelin | 1.3 | 0.108230064 | 0.044713728 | 1.36 | 0.075537285 | 0.031209427 |
| | (d18:0/18:0, | | | | | | |
| | d19:0/17:0)* | | | | | | |
| | sphingomyelin | 0.98 | 0.890179797 | 0.242024 | 0.95 | 0.974925045 | 0.253418078 |
| | (d18:1/19:0, | | | | | | |
| | d19:1/18:0)* | | | | | | |
| | heptadecasphingosine | 0.77 | 0.333040379 | 0.115333587 | 0.79 | 0.434383972 | 0.136113114 |
| | (d17:1) | | | | | | |
| | hexadecasphingosine | 0.65 | 0.123257998 | 0.049887948 | 0.66 | 0.165599534 | 0.061638795 |
| | (d16:1)* | | | | | | |
| | N-stearoyl- | 0.9 | 0.435541008 | 0.142270995 | 0.92 | 0.531164257 | 0.159738709 |
| | sphinganine | | | | | | |
| | (d18:0/18:0)* | | | | | | |
| | sphingadienine | 0.7 | 0.218738872 | 0.08113415 | 0.71 | 0.281388756 | 0.09659274 |
| | lactosyl-N-behenoyl- | 0.67 | 0.126882726 | 0.051115432 | 0.64 | 0.10808452 | 0.042761025 |
| | sphingosine | | | | | | |
| | (d18:1/22:0)* | | | | | | |
| | hexadecasphinganine | 0.46 | 0.011566051 | 0.006747807 | 0.45 | 0.015527536 | 0.008156592 |
| | (d16:0)* | | | | | | |
| | N-(2- | 2.02 | 1.25886E−06 | 2.88577E−06 | 2.3 | 1.255E−08 | 5.092E−08 |
| | hydroxypalmitoyl)- | | | | | | |
| | sphingosine | | | | | | |
| | (d18:1/16:0(2OH)) | | | | | | |
| | N-oleoyl-sphingosine | 0.65 | 0.041118671 | 0.019688032 | 0.66 | 0.061616455 | 0.026322258 |
| | (d18:1/18:1)* | | | | | | |
| Ceramides | N-palmitoyl- | 1.07 | 0.430492931 | 0.141338711 | 1.1 | 0.316236975 | 0.105252537 |
| | sphingosine | | | | | | |
| | (d18:1/16:0) | | | | | | |
| | N-stearoyl- | 0.98 | 0.608546652 | 0.18393889 | 1.01 | 0.491788796 | 0.149809828 |
| | sphingosine | | | | | | |
| | (d18:1/18:0)* | | | | | | |
| | ceramide (d16:1/24:1, | 0.95 | 0.801239293 | 0.224888909 | 0.99 | 0.655062707 | 0.184989421 |
| | d18:1/22:1)* | | | | | | |
| | ceramide (d18:1/14:0, | 0.91 | 0.765067499 | 0.217541382 | 0.97 | 0.580149049 | 0.167653074 |
| | d16:1/16:0)* | | | | | | |
| | ceramide (d18:1/17:0, | 0.94 | 0.777721896 | 0.220413726 | 0.96 | 0.661209696 | 0.186098733 |
| | d17:1/18:0)* | | | | | | |
| | ceramide (d18:1/20:0, | 1.01 | 0.546813724 | 0.170041493 | 1.04 | 0.433291923 | 0.135940213 |
| | d16:1/22:0, | | | | | | |
| | d20:1/18:0)* | | | | | | |
| | ceramide (d18:2/24:1, | 1.01 | 0.541903718 | 0.168717181 | 1.06 | 0.413770531 | 0.131288925 |
| | d18:1/24:2)* | | | | | | |
| | glycosyl-N-palmitoyl- | 1.15 | 0.476609104 | 0.152265445 | 1.19 | 0.356943497 | 0.116338617 |
| | sphingosine | | | | | | |
| | (d18:1/16:0) | | | | | | |
| | glycosyl-N-stearoyl- | 0.95 | 0.615403157 | 0.185147168 | 0.93 | 0.555481893 | 0.163546009 |
| | sphingosine | | | | | | |
| | (d18:1/18:0) | | | | | | |
| | glycosyl-N-(2- | 1.28 | 0.082588717 | 0.035517804 | 1.3 | 0.069298449 | 0.028773495 |
| | hydroxynervonoyl)- | | | | | | |
| | sphingosine | | | | | | |
| | (d18:1/24:1(2OH))* | | | | | | |
| | lactosyl-N-palmitoyl- | 0.62 | 0.018784961 | 0.010265792 | 0.63 | 0.025443512 | 0.012503996 |
| | sphingosine | | | | | | |
| | (d18:1/16:0) | | | | | | |
| | lactosyl-N-stearoyl- | 0.58 | 0.149777576 | 0.059135177 | 0.56 | 0.113725947 | 0.044572424 |
| | sphingosine | | | | | | |
| | (d18:1/18:0)* | | | | | | |
| | lactosyl-N-nervonoyl- | 0.65 | 0.019022779 | 0.010373871 | 0.64 | 0.020931849 | 0.010523064 |
| | sphingosine | | | | | | |
| | (d18:1/24:1)* | | | | | | |
| | glycosyl ceramide | 0.86 | 0.125670484 | 0.050785036 | 0.85 | 0.110973374 | 0.043561466 |
| | (d18:1/20:0, | | | | | | |
| | d16:1/22:0)* | | | | | | |
| | glycosyl ceramide | 0.77 | 0.042143365 | 0.019978484 | 0.75 | 0.033539909 | 0.015628232 |
| | (d18:2/24:1, | | | | | | |
| | d18:1/24:2)* | | | | | | |
| | eicosanoylsphingosine | 0.83 | 0.222528989 | 0.082112635 | 0.83 | 0.271763942 | 0.094188295 |
| | (d20:1)* | | | | | | |
| Mevalonate | 3-hydroxy-3- | 2.05 | 7.21447E−05 | 8.3429E−05 | 2.13 | 5.0168E−05 | 5.68612E−05 |
| Metabolism | methylglutarate | | | | | | |
| | mevalonolactone | 1.24 | 0.051494339 | 0.023504319 | 1.26 | 0.045247384 | 0.020150543 |
| Sterol | lanosterol | 1 | 0.753465092 | 0.215187161 | 0.98 | 0.697857317 | 0.193172133 |
| | desmosterol | 0.69 | 0.058510702 | 0.026176842 | 0.67 | 0.05871962 | 0.025342905 |
| | cholesterol | 0.56 | 0.03188215 | 0.015881984 | 0.54 | 0.022646141 | 0.011283519 |
| | 7-ketocholesterol | 1.36 | 0.09748139 | 0.040836457 | 1.35 | 0.105518733 | 0.041943781 |
| | 4-cholesten-3-one | 0.88 | 0.600811297 | 0.182238749 | 0.85 | 0.499653319 | 0.151654733 |
| | 7-hydroxycholesterol | 2.42 | 0.000405241 | 0.000380334 | 2.83 | 8.35144E−05 | 8.57758E−05 |
| | (alpha or beta) | | | | | | |
| Pregnenolone | pregnenolone sulfate | 1.45 | 0.082680464 | 0.035517804 | 1.48 | 0.065519088 | 0.027385021 |
| Steroids | 17alpha- | 2.19 | 0.000735684 | 0.000633119 | 2.35 | 0.000324218 | 0.000286243 |
| | hydroxypregnenolone | | | | | | |
| | 3-sulfate | | | | | | |
| | 21- | 1.61 | 0.020532309 | 0.010988867 | 1.62 | 0.019060409 | 0.00974785 |
| | hydroxypregnenolone | | | | | | |
| | disulfate | | | | | | |
| | 21- | 1.61 | 0.028569748 | 0.014596844 | 1.72 | 0.018606548 | 0.009554577 |
| | hydroxypregnanolone | | | | | | |
| | disulfate | | | | | | |
| Progestin | 5alpha-pregnan- | 1.41 | 0.218937603 | 0.08113415 | 1.37 | 0.263869377 | 0.092152272 |
| Steroids | 3beta,20alpha-diol | | | | | | |
| | disulfate | | | | | | |
| | 5alpha-pregnan- | 1.5 | 0.12888716 | 0.051761928 | 1.65 | 0.072656159 | 0.030068418 |
| | 3(alpha or | | | | | | |
| | beta),20beta-diol | | | | | | |
| | disulfate | | | | | | |
| | pregnanediol-3- | 1.33 | 0.182400239 | 0.069997437 | 1.32 | 0.202683552 | 0.073804388 |
| | glucuronide | | | | | | |
| | pregnen-diol disulfate | 1.41 | 0.116761085 | 0.047536723 | 1.46 | 0.090239355 | 0.036681497 |
| | C21H34O8S2* | | | | | | |
| | pregn steroid | 1.36 | 0.187658726 | 0.071591176 | 1.36 | 0.196279781 | 0.071784198 |
| | monosulfate | | | | | | |
| | C21H34O5S* | | | | | | |
| Androgenic | 11- | 3.8 | 0.002065584 | 0.00155541 | 3.86 | 0.001695104 | 0.001222116 |
| Steroids | ketoetiocholanolone | | | | | | |
| | sulfate | | | | | | |
| | dehydroisoandrosterone | 1.35 | 0.117395378 | 0.047664037 | 1.43 | 0.07690431 | 0.031670228 |
| | sulfate (DHEA-S) | | | | | | |
| | 16a-hydroxy DHEA 3- | 2.18 | 0.001343 | 0.001057403 | 2.25 | 0.000960324 | 0.000747927 |
| | sulfate | | | | | | |
| | androsterone sulfate | 1.36 | 0.05057369 | 0.023180913 | 1.45 | 0.01518077 | 0.00804159 |
| | 5alpha-androstan- | 1.73 | 0.017354753 | 0.009626359 | 1.8 | 0.011021652 | 0.006080709 |
| | 3alpha,17alpha-diol | | | | | | |
| | disulfate | | | | | | |
| | androstenediol | 2.92 | 0.62917384 | 0.188197249 | 2.9 | 0.650139829 | 0.183805494 |
| | (3beta,17beta) | | | | | | |
| | monosulfate (1) | | | | | | |
| | androstenediol | 1.91 | 0.025457843 | 0.01329537 | 1.85 | 0.039010024 | 0.017781929 |
| | (3beta,17beta) | | | | | | |
| | disulfate (1) | | | | | | |
| | androstenediol | 1.68 | 0.0259653 | 0.013478864 | 1.63 | 0.038983074 | 0.017781929 |
| | (3beta,17beta) | | | | | | |
| | disulfate (2) | | | | | | |
| | androstenediol | 1.49 | 0.115652628 | 0.0471783 | 1.53 | 0.085191984 | 0.03485502 |
| | (3alpha, 17alpha) | | | | | | |
| | monosulfate (2) | | | | | | |
| | 5alpha-androstan- | 3.53 | 0.000713525 | 0.000620231 | 3.48 | 0.000852192 | 0.000676425 |
| | 3alpha,17beta-diol | | | | | | |
| | disulfate | | | | | | |
| | andro steroid | 2 | 0.002220871 | 0.001657885 | 2.06 | 0.001695041 | 0.001222116 |
| | monosulfate | | | | | | |
| | C19H28O6S (1)* | | | | | | |
| Primary | cholate | 3.06 | 0.002573777 | 0.001898437 | 3.21 | 0.002251349 | 0.001552 |
| Bile Acid | glycocholate | 0 | 1.356E−08 | 6.889E−08 | 0 | 1.21E−09 | 6.93E−09 |
| Metabolism | taurocholate | 0 | 1.64E−09 | 1.287E−08 | 0 | 1.3E−10 | 9.5E−10 |
| | chenodeoxycholate | 4.79 | 0.002647257 | 0.001931649 | 4.85 | 0.002628319 | 0.001787385 |
| | glycochenodeoxycholate | 0.02 | 3.54933E−06 | 6.80057E−06 | 0.01 | 1.29966E−06 | 2.42058E−06 |
| | taurochenodeoxycholate | 0 | 3.101E−08 | 1.3909E−07 | 0 | 7.18E−09 | 3.245E−08 |
| | cholate sulfate | 5.01 | 3.73288E−06 | 7.05803E−06 | 5.64 | 2.95107E−06 | 4.91749E−06 |
| | glycochenodeoxycholate | 0.02 | 6.80447E−06 | 1.15961E−05 | 0.02 | 4.37814E−06 | 6.97227E−06 |
| | sulfate | | | | | | |
| | glycocholate | 0.34 | 0.000548068 | 0.000494668 | 0.32 | 0.000488927 | 0.000417026 |
| | glucuronide (1) | | | | | | |
| | glycocholate sulfate | 0.09 | 1.56085E−05 | 2.32408E−05 | 0.09 | 1.09357E−05 | 1.58139E−05 |
| Secondary | glycolithocholate | 0.03 | 1.19646E−06 | 2.80758E−06 | 0.03 | 4.3209E−07 | 9.8839E−07 |
| Bile Acid | sulfate* | | | | | | |
| Metabolism | taurolithocholate 3- | 0.05 | 1.03249E−06 | 2.49956E−06 | 0.05 | 5.5168E−07 | 1.21766E−06 |
| | sulfate | | | | | | |
| | ursodeoxycholate | 1.93 | 0.750364469 | 0.215012811 | 1.91 | 0.811629203 | 0.218651525 |
| | tauroursodeoxycholate | 0.07 | 2.86503E−05 | 3.92215E−05 | 0.07 | 2.11966E−05 | 2.80948E−05 |
| | 7,12- | 1 | 0.594154259 | 0.18073343 | 0.96 | 0.563154242 | 0.164575783 |
| | diketolithocholate | | | | | | |
| | 7-ketolithocholate | 0.32 | 0.031244777 | 0.015746166 | 0.31 | 0.044691495 | 0.020005355 |
| | hyocholate | 1.39 | 0.143555227 | 0.057033749 | 1.49 | 0.106475401 | 0.042190753 |
| | glycohyocholate | 0.02 | 4.05802E−06 | 7.50839E−06 | 0.02 | 2.45926E−06 | 4.18104E−06 |
| | 3-dehydrocholate | 1.68 | 0.506081586 | 0.159094011 | 1.66 | 0.475047465 | 0.146483453 |
| | 12-dehydrocholate | 0.93 | 0.548355996 | 0.170112657 | 0.91 | 0.599336821 | 0.171954319 |
| | glycocholenate | 0.06 | 1.20308E−06 | 2.80758E−06 | 0.06 | 6.27E−08 | 1.9477E−07 |
| | sulfate* | | | | | | |
| | taurocholenate sulfate | 0.14 | 0.000129522 | 0.000138641 | 0.13 | 6.43368E−05 | 7.00792E−05 |
| | 7-ketodeoxycholate | 0.5 | 0.21783671 | 0.080957818 | 0.56 | 0.311549459 | 0.104382763 |
| | 3b-hydroxy-5- | 0.55 | 0.033607618 | 0.016519147 | 0.6 | 0.060674732 | 0.026008271 |
| | cholenoic acid | | | | | | |
| | ursodeoxycholate | 4.82 | 0.088862329 | 0.037673588 | 5.05 | 0.063268107 | 0.026790306 |
| | sulfate (1) | | | | | | |
| | ursocholate | 42.86 | 0.835833505 | 0.231810661 | 45.7 | 0.719610708 | 0.198756327 |
| Purine | inosine | 0.02 | 8.60E−14 | 5.55E−12 | 0.02 | 3.42E−13 | 8.60E−12 |
| Metabolism, | hypoxanthine | 1.63 | 0.002500728 | 0.001856101 | 2.15 | 0.000977079 | 0.000751837 |
| (Hypo)Xanthine/ | xanthine | 1.64 | 0.001518544 | 0.001174203 | 3.4 | 0.000212131 | 0.000196236 |
| Inosine | xanthosine | 0.8 | 0.429479789 | 0.141338711 | 0.78 | 0.404527253 | 0.128518109 |
| containing | N1-methylinosine | 0.22 | 0.000110938 | 0.000120734 | 0.21 | 9.07465E−05 | 9.28189E−05 |
| | 2′-deoxyinosine | 0.57 | 0.011685715 | 0.0068023 | 0.59 | 0.020219851 | 0.010278138 |
| | urate | 3.44 | 0.000114338 | 0.000123407 | 3.71 | 9.88505E−05 | 9.98899E−05 |
| | allantoin | 1.13 | 0.081965747 | 0.03532795 | 1.16 | 0.059924714 | 0.025818728 |
| Purine | adenosine 5′- | 1.67 | 0.058681874 | 0.026200348 | 1.78 | 0.034070523 | 0.015846132 |
| Metabolism, | monophosphate | | | | | | |
| Adenine | (AMP) | | | | | | |
| containing | adenosine | 0.65 | 0.009643679 | 0.005822989 | 0.63 | 0.009388672 | 0.005238046 |
| | adenine | 16.7 | 9E−11 | 1.62E−09 | 27.12 | 1E−11 | 1.5E−10 |
| | 1-methyladenine | 2.51 | 4.59913E−06 | 8.33106E−06 | 2.81 | 1.09294E−06 | 2.16539E−06 |
| | N6- | 1.48 | 0.003780656 | 0.002618521 | 1.47 | 0.004752794 | 0.002982266 |
| | dimethylallyladenine | | | | | | |
| | N6-carbamoyl- | 0.14 | 8.4674E−07 | 2.23813E−06 | 0.13 | 9.2643E−07 | 1.90723E−06 |
| | threonyladenosine | | | | | | |
| | 2′-deoxyadenosine | 2.38 | 0.029805075 | 0.015079285 | 2.36 | 0.028842168 | 0.013849635 |
| | N6-succinyladenosine | 0.47 | 0.269011361 | 0.096693681 | 0.56 | 0.416237848 | 0.131574044 |
| Purine | guanosine | 0.04 | 5.4E−10 | 5.8E−09 | 0.04 | 8.3E−10 | 0.000000005 |
| Metabolism, | guanine | 9.71 | 2.84191E−05 | 3.91573E−05 | 10.08 | 2.17788E−05 | 2.85413E−05 |
| Guanine | 1-methylguanine | 4.37 | 5.50293E−06 | 9.76342E−06 | 4.68 | 2.79187E−06 | 4.68323E−06 |
| containing | 7-methylguanine | 2.01 | 1.61483E−05 | 2.39028E−05 | 2.04 | 1.72537E−05 | 2.33406E−05 |
| | N1-methylguanosine | 0.97 | 0.166482259 | 0.064655077 | 0.97 | 0.146989217 | 0.055449976 |
| | N2,N2- | 0.06 | 0.000296031 | 0.000285066 | 0.06 | 0.00060386 | 0.000503119 |
| | dimethylguanosine | | | | | | |
| | N2,N2- | 1.88 | 0.005241622 | 0.003454885 | 1.93 | 0.004321279 | 0.002773754 |
| | dimethylguanine | | | | | | |
| | 8-hydroxyguanine | 14.47 | 0.041400144 | 0.019786231 | 14.3 | 0.043887506 | 0.019719441 |
| | 2′-deoxyguanosine | 0.18 | 9.9088E−07 | 2.45895E−06 | 0.18 | 1.06134E−06 | 2.11945E−06 |
| Pyrimidine | dihydroorotate | 0.07 | 8.6382E−07 | 2.2602E−06 | 0.07 | 2.1101E−06 | 3.66165E−06 |
| Metabolism, | orotate | 1.65 | 0.000256695 | 0.000254763 | 1.85 | 3.01548E−05 | 3.66545E−05 |
| Orotate | orotidine | 2.34 | 5.712E−08 | 2.3022E−07 | 2.4 | 4.399E−08 | 1.4563E−07 |
| containing | | | | | | | |
| Pyrimidine | uridine | 0.11 | 1.35E−09 | 1.209E−08 | 0.11 | 1.92E−09 | 1.029E−08 |
| Metabolism, | uracil | 1.86 | 0.00804599 | 0.004986131 | 1.87 | 0.008175798 | 0.004686055 |
| Uracil | pseudouridine | 4.81 | 3.9417E−07 | 1.18725E−06 | 6.61 | 7.486E−08 | 2.1784E−07 |
| containing | 2′-O-methyluridine | 4.02 | 1.63E−09 | 1.287E−08 | 7.25 | 2E−11 | 1.8E−10 |
| | 5-methyluridine | 0.2 | 3.90109E−06 | 7.32264E−06 | 0.19 | 5.96588E−06 | 9.32374E−06 |
| | (ribothymidine) | | | | | | |
| | 2′-deoxyuridine | 1.99 | 0.000261444 | 0.000258487 | 2 | 0.000328142 | 0.000288694 |
| | 3-ureidopropionate | 0.71 | 0.022860618 | 0.012060546 | 0.69 | 0.014949246 | 0.00795605 |
| Pyrimidine | cytidine | 1.9 | 0.002514508 | 0.001860996 | 2.13 | 0.000938458 | 0.000735617 |
| Metabolism, | cytosine | 18.73 | 5.7E−10 | 5.86E−09 | 43.05 | 4E−11 | 3.9E−10 |
| Cytidine | 3-methylcytidine | 0.95 | 0.802194173 | 0.224888909 | 0.99 | 0.920016399 | 0.242909439 |
| containing | 2′-deoxycytidine | 2.18 | 0.000969653 | 0.000810243 | 2.23 | 0.00102677 | 0.000782891 |
| | 2′-O-methylcytidine | 25.49 | 7.58E−09 | 4.361E−08 | 25.04 | 7.44E−09 | 3.284E−08 |
| Pyrimidine | thymidine | 2.65 | 3.88378E−05 | 5.00518E−05 | 2.66 | 5.25123E−05 | 5.89868E−05 |
| Metabolism, | thymine | 0.53 | 0.017818718 | 0.009862593 | 0.53 | 0.025262595 | 0.012439381 |
| Thymine | 5,6-dihydrothymine | 0.53 | 0.032296542 | 0.016026771 | 0.51 | 0.023383284 | 0.011604843 |
| containing | | | | | | | |
| Purine and | methylphosphate | 1.45 | 0.018178109 | 0.010011583 | 1.63 | 0.008749176 | 0.004924945 |
| Pyrimidine | | | | | | | |
| Metabolism | | | | | | | |
| Nicotinate | quinolinate | 0.89 | 0.880288418 | 0.24002799 | 0.92 | 0.990791504 | 0.255134788 |
| and | nicotinate | 1.64 | 0.031367282 | 0.015777208 | 1.71 | 0.021528167 | 0.010788843 |
| Nicotinamide | nicotinate | 0.32 | 0.006589736 | 0.004194055 | 0.5 | 0.011934341 | 0.006542255 |
| Metabolism | ribonucleoside | | | | | | |
| | nicotinamide | 3.91 | 6.32389E−05 | 7.5143E−05 | 3.85 | 7.90931E−05 | 8.22366E−05 |
| | nicotinamide | 5.27 | 0.001545059 | 0.00119115 | 5.48 | 0.00096308 | 0.000747927 |
| | ribonucleotide (NMN) | | | | | | |
| | nicotinamide riboside | 4.92 | 5.337E−08 | 2.1942E−07 | 6.41 | 6.76E−09 | 3.15E−08 |
| | nicotinamide adenine | 3.91 | 0.187260001 | 0.071544431 | 3.9 | 0.183031962 | 0.067232318 |
| | dinucleotide (NAD+) | | | | | | |
| | 1-methylnicotinamide | 2 | 0.003650614 | 0.002555787 | 2.07 | 0.002505246 | 0.001708307 |
| | trigonelline (N′- | 0.04 | 0.39282002 | 0.132665731 | 0.04 | 0.343840104 | 0.112651514 |
| | methylnicotinate) | | | | | | |
| | N1-Methyl-2- | 1.21 | 0.06749847 | 0.029534711 | 1.21 | 0.069000353 | 0.028697077 |
| | pyridone-5- | | | | | | |
| | carboxamide | | | | | | |
| Riboflavin | riboflavin (Vitamin B2) | 2.91 | 1.74554E−06 | 3.83186E−06 | 3.02 | 1.21575E−06 | 2.35312E−06 |
| Metabolism | | | | | | | |
| Pantothenate | pantothenate | 0.73 | 0.11432551 | 0.046710487 | 0.72 | 0.124636876 | 0.04795242 |
| and CoA | pantetheine | 1.42 | 0.496412463 | 0.157138227 | 1.69 | 0.326564284 | 0.108117704 |
| Metabolism | | | | | | | |
| Ascorbate | threonate | 1.97 | 0.008476158 | 0.00520292 | 1.92 | 0.01217579 | 0.006645657 |
| and Aldarate | gulonate* | 5.8 | 4.7E−10 | 5.25E−09 | 7.69 | 1E−11 | 1.6E−10 |
| Metabolism | | | | | | | |
| Tocopherol | alpha-tocopherol | 1.49 | 0.001032873 | 0.000857537 | 1.53 | 0.000925938 | 0.000728071 |
| Metabolism | delta-tocopherol | 1.16 | 0.339013561 | 0.116933147 | 1.22 | 0.252887429 | 0.089119092 |
| | alpha-tocotrienol | 2.05 | 0.061603659 | 0.027324622 | 2.11 | 0.048435824 | 0.021381273 |
| | gamma-tocotrienol | 1.65 | 0.027669673 | 0.014249399 | 1.75 | 0.01824145 | 0.009405486 |
| | alpha-CEHC sulfate | 2.73 | 0.000657426 | 0.00058321 | 2.63 | 0.00123533 | 0.000924437 |
| | gamma- | 1.49 | 0.022492029 | 0.011890307 | 1.65 | 0.008743213 | 0.004924945 |
| | tocopherol/beta- | | | | | | |
| | tocopherol | | | | | | |
| Tetrahydrobiopterin | biopterin | 2.1 | 1.11385E−05 | 1.77011E−05 | 2.33 | 1.29046E−06 | 2.42058E−06 |
| Metabolism | dihydrobiopterin | 1.78 | 0.001679378 | 0.00129086 | 1.78 | 0.002048094 | 0.00143548 |
| Hemoglobin | protoporphyrin IX | 0.39 | 0.780641435 | 0.220999357 | 0.45 | 0.986710133 | 0.255134788 |
| and | heme | 1 | 0.432287242 | 0.141716356 | 0.97 | 0.49840692 | 0.151590551 |
| Porphyrin | bilirubin (Z, Z) | 0.95 | 0.801856832 | 0.224888909 | 0.94 | 0.727813708 | 0.200353921 |
| Metabolism | bilirubin (E, E)* | 0.67 | 0.091899057 | 0.038833907 | 0.65 | 0.063350919 | 0.026790306 |
| | bilirubin (E, Z or Z, E)* | 1.07 | 0.833124924 | 0.231555297 | 1.03 | 0.968758548 | 0.252598029 |
| | biliverdin | 1.15 | 0.28000912 | 0.099769609 | 1.15 | 0.28477571 | 0.097292337 |
| | I-urobilinogen | 0.82 | 0.173695354 | 0.066854962 | 0.81 | 0.167457422 | 0.062054938 |
| | D-urobilin | 0.43 | 0.108962612 | 0.044802006 | 0.41 | 0.097691652 | 0.03926674 |
| Thiamine | thiamin (Vitamin B1) | 6.07 | 4.69E−09 | 3.214E−08 | 7.49 | 5E−10 | 3.2E−09 |
| Metabolism | hydroxymethylpyrimidine | 1.67 | 0.000892015 | 0.000755112 | 1.65 | 0.00095488 | 0.000746165 |
| Vitamin B6 | pyridoxamine | 1.31 | 0.102509804 | 0.042622396 | 1.3 | 0.100921552 | 0.040179873 |
| Metabolism | pyridoxal | 1.51 | 0.005464934 | 0.003574788 | 1.56 | 0.004468826 | 0.002839488 |
| | pyridoxate | 1.64 | 0.086318687 | 0.036775768 | 1.58 | 0.121457122 | 0.047016614 |
| Benzoate | hippurate | 2.35 | 0.002706988 | 0.001953226 | 2.24 | 0.005450106 | 0.003344744 |
| Metabolism | 2-hydroxyhippurate | 1.34 | 0.108796954 | 0.044802006 | 1.31 | 0.128558353 | 0.049385644 |
| | (salicylurate) | | | | | | |
| | 3-hydroxyhippurate | 3.4 | 6.49313E−05 | 7.68017E−05 | 3.3 | 0.000122024 | 0.000119468 |
| | 4-hydroxyhippurate | 2.3 | 2.66871E−05 | 3.73672E−05 | 2.26 | 5.77958E−05 | 6.40638E−05 |
| | benzoate | 0.34 | 0.000670403 | 0.000592691 | 0.33 | 0.000227597 | 0.000208246 |
| | 4-hydroxybenzoate | 0.49 | 0.000693034 | 0.000604447 | 0.48 | 0.000746197 | 0.000605667 |
| | methyl-4- | 8.89 | 0.376854597 | 0.128108915 | 8.69 | 0.39410691 | 0.125683645 |
| | hydroxybenzoate | | | | | | |
| | p-cresol sulfate | 0.89 | 0.303598923 | 0.106977353 | 0.86 | 0.251772152 | 0.088850502 |
| | 3-phenylpropionate | 2.53 | 0.331418652 | 0.114925619 | 2.44 | 0.291810381 | 0.099222218 |
| | (hydrocinnamate) | | | | | | |
| Xanthine | caffeine | 2.93 | 0.013778019 | 0.007861247 | 2.82 | 0.018595655 | 0.009554577 |
| Metabolism | theobromine | 1.61 | 0.269981497 | 0.096728891 | 1.6 | 0.259861262 | 0.09113373 |
| | 1-methylurate | 3.09 | 5.98234E−05 | 7.23162E−05 | 3.05 | 9.5669E−05 | 9.70647E−05 |
| | 7-methylurate | 3.36 | 0.008948508 | 0.005428543 | 3.54 | 0.006837078 | 0.004038338 |
| | 1,3-dimethylurate | 6.54 | 4.89569E−05 | 6.15612E−05 | 6.35 | 7.0208E−05 | 7.51728E−05 |
| | 1,7-dimethylurate | 4.87 | 0.003045202 | 0.002185091 | 4.69 | 0.004671237 | 0.002945783 |
| | 3,7-dimethylurate | 2.99 | 0.004385192 | 0.002965858 | 3.58 | 0.001573785 | 0.001147805 |
| | 1,3,7-trimethylurate | 3.96 | 0.031827151 | 0.015881984 | 3.87 | 0.037743767 | 0.017330313 |
| | 1-methylxanthine | 3.44 | 0.044187061 | 0.020660739 | 3.53 | 0.0325649 | 0.01523133 |
| | 3-methylxanthine | 2.25 | 0.008163535 | 0.0050469 | 2.35 | 0.005076107 | 0.003138182 |
| | 5-acetylamino-6- | 3.86 | 0.003779893 | 0.002618521 | 3.73 | 0.00494944 | 0.003074984 |
| | amino-3-methyluracil | | | | | | |
| | 5-acetylamino-6- | 3.62 | 0.04189949 | 0.019914653 | 3.58 | 0.045100658 | 0.020120812 |
| | formylamino-3- | | | | | | |
| | methyluracil | | | | | | |
| Food | piperidine | 0.08 | 0.010245207 | 0.00608688 | 0.08 | 0.008874339 | 0.004984249 |
| Component/ | 2-piperidinone | 0.98 | 0.037325493 | 0.018140065 | 0.94 | 0.011257928 | 0.006184929 |
| Plant | erythrose | 6.43 | 7.46E−09 | 4.361E−08 | 8.36 | 6.7E−10 | 4.21E−09 |
| | sucralose | 0.84 | 0.329876801 | 0.114698043 | 0.83 | 0.330564931 | 0.109011914 |
| | shikimate | 1.76 | 0.000270965 | 0.00026587 | 3.57 | 1.05113E−06 | 2.11587E−06 |
| | 3-dehydroshikimate | 0.32 | 0.84193423 | 0.233252905 | 0.32 | 0.991565754 | 0.255134788 |
| | diaminopimelate | 0.55 | 0.001409269 | 0.001096251 | 0.56 | 0.002107921 | 0.001469227 |
| | 2,3- | 0.64 | 0.627679351 | 0.187967273 | 0.62 | 0.621668972 | 0.177753533 |
| | dihydroxyisovalerate | | | | | | |
| | 2-isopropylmalate | 0.32 | 0.289629479 | 0.102773325 | 0.31 | 0.271487033 | 0.094188295 |
| | gluconate | 6.07 | 4.133E−08 | 1.7843E−07 | 6.68 | 1.588E−08 | 5.963E−08 |
| | 2-acetolactate | 3.9 | 1.26363E−05 | 1.96004E−05 | 3.9 | 1.26363E−05 | 1.77627E−05 |
| | beta-cryptoxanthin | 0.8 | 0.505505762 | 0.159094011 | 0.8 | 0.569292104 | 0.16576167 |
| | ergothioneine | 0.86 | 0.482234517 | 0.153648475 | 0.87 | 0.561788604 | 0.164367594 |
| | erythritol | 3.87 | 4.7818E−07 | 1.37629E−06 | 5.78 | 1.366E−08 | 5.371E−08 |
| | fucitol | 4.11 | 1.215E−08 | 6.309E−08 | 8.3 | 2E−11 | 2.1E−10 |
| | histidinol | 5.42 | 7.62E−13 | 3E−11 | 5.34 | 1.94E−12 | 3E−11 |
| | methyl indole-3- | 0.13 | 0.001064685 | 0.000881124 | 0.13 | 0.000821596 | 0.000656281 |
| | acetate | | | | | | |
| | quinate | 4.39 | 2.32121E−06 | 4.96924E−06 | 4.38 | 4.14722E−06 | 6.6892E−06 |
| | stachydrine | 0.63 | 0.547959146 | 0.170112657 | 0.61 | 0.522466996 | 0.157628424 |
| | tyrosol | 0.52 | 0.086159277 | 0.036768326 | 0.51 | 0.083085247 | 0.034048444 |
| | pyrraline | 4.37 | 3.2522E−07 | 9.911E−07 | 4.75 | 1.1476E−07 | 3.173E−07 |
| | carotene diol (1) | 0.39 | 0.005398817 | 0.003540479 | 0.38 | 0.00607114 | 0.003672135 |
| | carotene diol (2) | 0.41 | 0.021534017 | 0.011430513 | 0.4 | 0.019404439 | 0.009903664 |
| | carotene diol (3) | 0.49 | 0.027602234 | 0.014242985 | 0.48 | 0.032584521 | 0.01523133 |
| Drug | 2- | 0.96 | 0.799951531 | 0.224888909 | 0.94 | 0.765239956 | 0.207935372 |
| | hydroxyacetaminophen | | | | | | |
| | sulfate* | | | | | | |
| | 2- | 1.61 | 0.879730754 | 0.24002799 | 1.57 | 0.923383604 | 0.243542919 |
| | methoxyacetaminophen | | | | | | |
| | sulfate* | | | | | | |
| | 3-(cystein-S- | 0.14 | 0.271181834 | 0.09702475 | 0.14 | 0.257920523 | 0.090765662 |
| | yl)acetaminophen* | | | | | | |
| | 3-(N-acetyl-L-cystein- | 0.96 | 0.953733645 | 0.255524565 | 0.94 | 0.991669169 | 0.255134788 |
| | S-yl) acetaminophen | | | | | | |
| | 4-acetaminophen | 0.58 | 0.80644016 | 0.225834829 | 0.55 | 0.857808409 | 0.229576139 |
| | sulfate | | | | | | |
| | 4-acetamidophenol | 0.82 | 0.445147428 | 0.143598145 | 0.81 | 0.419145489 | 0.132326918 |
| | 4- | 1.19 | 0.464761181 | 0.149182135 | 1.19 | 0.473078338 | 0.146055251 |
| | acetamidophenylglucuronide | | | | | | |
| | N- | 1.75 | 0.576826678 | 0.176785257 | 1.7 | 0.672797973 | 0.188097874 |
| | carbamoylglutamate | | | | | | |
| | gabapentin | 1 | | | 1 | | |
| | salicylate | 1.01 | 0.984876271 | 0.261392042 | 0.99 | 0.934534413 | 0.245896056 |
| | S-carboxymethyl-L- | 1.16 | 0.417088038 | 0.137983285 | 1.22 | 0.319143617 | 0.106079632 |
| | cysteine | | | | | | |
| | 2-acetamidophenol | 1.38 | 0.212270333 | 0.079344453 | 1.34 | 0.240366275 | 0.085666407 |
| | sulfate | | | | | | |
| Chemical | 1,3-propanediol | 0.09 | 0.01636284 | 0.009154575 | 0.09 | 0.015731691 | 0.008246618 |
| | sulfate* | 0.54 | 0.063226262 | 0.027901009 | 0.53 | 0.064115148 | 0.027067996 |
| | O-sulfo-L-tyrosine | 2.09 | 0.002579754 | 0.001898437 | 2.31 | 0.000984778 | 0.000755451 |
| | 5-aminoimidazole-4- | 1.83 | 0.015422695 | 0.008741875 | 1.81 | 0.014637497 | 0.007819656 |
| | carboxamide | | | | | | |
| | dexpanthenol | 0.02 | 0.653679066 | 0.193074754 | 0.02 | 0.987650399 | 0.255134788 |
| | azeloylcarnitine | 1.9 | 0.020148646 | 0.010805858 | 1.83 | 0.032462802 | 0.01523133 |
| | (C9-DC) | | | | | | |
| | succinimide | 1 | 0.907230613 | 0.245564885 | 0.98 | 0.734625123 | 0.201137009 |
| | triethanolamine | 4.37 | 0.061101526 | 0.027195032 | 4.26 | 0.06517095 | 0.027330308 |
| | thioproline | 2.45 | 0.04326602 | 0.020414329 | 2.43 | 0.044762356 | 0.020005355 |
| N/A | X - 10457 | 2.57 | 8.652E−08 | 3.2479E−07 | 2.82 | 7.22E−09 | 3.245E−08 |
| | X - 11261 | 3.06 | 1.95835E−05 | 2.81824E−05 | 3.22 | 1.58675E−05 | 2.18173E−05 |
| | X - 11299 | 1.36 | 0.415863392 | 0.137754072 | 1.32 | 0.489991277 | 0.149624551 |
| | X - 11357 | 35.35 | 6.70E−14 | 5.55E−12 | 64.12 | 2.00E−15 | 1.63E−13 |
| | X - 11429 | 0.09 | 1.64068E−05 | 2.41474E−05 | 0.1 | 4.382E−05 | 5.15229E−05 |
| | X - 11440 | 1.8 | 0.028527965 | 0.014596844 | 1.77 | 0.03573716 | 0.016469072 |
| | X - 11442 | 0.5 | 0.131052265 | 0.052468755 | 0.48 | 0.099849325 | 0.039865419 |
| | X - 11491 | 2.21 | 0.002783966 | 0.002003189 | 2.14 | 0.004777955 | 0.002990596 |
| | X - 11530 | 0.81 | 0.386693658 | 0.130938126 | 0.78 | 0.268775954 | 0.093410042 |
| | X - 11540 | 1.31 | 0.063932318 | 0.028116785 | 1.3 | 0.062364284 | 0.026506718 |
| | X - 11564 | 0.74 | 0.161217162 | 0.062881917 | 0.71 | 0.109374409 | 0.043135691 |
| | X - 11648 | 2.85 | 9.90358E−06 | 1.62366E−05 | 3.06 | 6.11644E−06 | 9.50003E−06 |
| | X - 11795 | 0.01 | 6.13228E−05 | 7.35409E−05 | 0.02 | 0.000134019 | 0.000130199 |
| | X - 11838 | 3.35 | 0.553962188 | 0.171188673 | 3.24 | 0.590480426 | 0.170189587 |
| | X - 12013 | 2.13 | 0.042188041 | 0.019978484 | 2.1 | 0.047109215 | 0.020832209 |
| | X - 12026 | 2.09 | 8.06177E−05 | 9.24022E−05 | 2.16 | 4.80988E−05 | 5.50115E−05 |
| | X - 12100 | 2.43 | 2.1916E−05 | 3.1022E−05 | 2.49 | 2.12147E−05 | 2.80948E−05 |
| | X - 12101 | 0.24 | 0.016870149 | 0.00941806 | 0.23 | 0.020952615 | 0.010523064 |
| | X - 12117 | 1.63 | 0.013654299 | 0.007807854 | 1.64 | 0.011982539 | 0.006554397 |
| | X - 12127 | 1.84 | 0.003762434 | 0.002618521 | 1.86 | 0.003300674 | 0.002174107 |
| | X - 12170 | 1.04 | 0.611143736 | 0.184293792 | 1.01 | 0.655987782 | 0.185042982 |
| | X - 12199 | 1.21 | 0.239612628 | 0.087791161 | 1.21 | 0.22170384 | 0.079806407 |
| | X - 12206 | 1.12 | 0.820792539 | 0.229110994 | 1.07 | 0.785299167 | 0.212696893 |
| | X - 12216 | 1.44 | 0.201539086 | 0.076324451 | 1.39 | 0.240905898 | 0.085737288 |
| | X - 12410 | 2.57 | 5.42979E−05 | 6.72972E−05 | 2.53 | 0.000109398 | 0.000108372 |
| | X - 12411 | 0.63 | 0.030440805 | 0.015370899 | 0.62 | 0.029375235 | 0.01402531 |
| | X - 12442 | 0.98 | 0.984285939 | 0.261392042 | 1.07 | 0.813518737 | 0.218926166 |
| | X - 12472 | 0.49 | 0.579914503 | 0.177144888 | 0.48 | 0.547224076 | 0.162755831 |
| | X - 12565 | 3.4 | 7.48E−12 | 2.8E−10 | 3.51 | 7.08E−12 | 1.1E−10 |
| | X - 12680 | 3.83 | 0.000360059 | 0.000341642 | 3.75 | 0.000438871 | 0.000376887 |
| | X - 12688 | 1.68 | 0.003241184 | 0.002312904 | 1.76 | 0.001626123 | 0.001182549 |
| | X - 12695 | 0.78 | 0.73246477 | 0.211051069 | 1.07 | 0.435316069 | 0.136235526 |
| | X - 12708 | 3.52 | 1.17936E−06 | 2.80273E−06 | 3.48 | 1.94022E−06 | 3.39024E−06 |
| | X - 12721 | 3.02 | 2.7502E−07 | 8.5832E−07 | 3.19 | 1.351E−07 | 3.6062E−07 |
| | X - 12740 | 8.39 | 3.09863E−05 | 4.15885E−05 | 8.11 | 4.57301E−05 | 5.32467E−05 |
| | X - 12798 | 2.4 | 0.00041783 | 0.000390733 | 2.6 | 0.000221913 | 0.000203796 |
| | X - 12822 | 2.59 | 3.60277E−05 | 4.66625E−05 | 2.57 | 4.59209E−05 | 5.32467E−05 |
| | X - 12844 | 0.46 | 0.083064008 | 0.035623489 | 0.44 | 0.051264876 | 0.022511634 |
| | X - 12879 | 0.49 | 3.46031E−06 | 6.73944E−06 | 0.47 | 4.0439E−07 | 9.4214E−07 |
| | X - 13007 | 0.61 | 0.096623425 | 0.040565588 | 0.6 | 0.108603973 | 0.042899082 |
| | X - 13431 | 2.62 | 0.000179755 | 0.000184774 | 2.69 | 0.000164631 | 0.000157507 |
| | X - 13529 | 7.05 | 6.9E−10 | 6.58E−09 | 25.02 | 3.29E−13 | 8.60E−12 |
| | X - 13553 | 2.43 | 9.40002E−05 | 0.000105409 | 2.46 | 9.13361E−05 | 9.30438E−05 |
| | X - 13728 | 2.53 | 0.001329514 | 0.001049977 | 2.83 | 0.000560162 | 0.000471394 |
| | X - 13729 | 2.38 | 0.491350484 | 0.155977421 | 2.29 | 0.555920483 | 0.163546009 |
| | X - 13737 | 1.87 | 0.000472819 | 0.000437418 | 2.02 | 0.000158543 | 0.000153432 |
| | X - 13844 | 5.05 | 1.55897E−06 | 3.48128E−06 | 5.11 | 1.64145E−06 | 2.90858E−06 |
| | X - 14056 | 7.68 | 5E−11 | 1.11E−09 | 14.64 | 3.70E−14 | 1.54E−12 |
| | X - 14095 | 0.11 | 0.004092223 | 0.002803432 | 0.11 | 0.008494761 | 0.004835825 |
| | X - 14096 | 0.13 | 0.016096256 | 0.009075326 | 0.19 | 0.031499078 | 0.014842201 |
| | X - 14141 | 0.61 | 0.003359362 | 0.0023841 | 0.6 | 0.002889501 | 0.001928516 |
| | X - 14254 | 1.1 | 0.067353413 | 0.029521107 | 1.11 | 0.060599005 | 0.026008271 |
| | X - 14263 | 0.42 | 0.010161423 | 0.006050981 | 0.4 | 0.003318918 | 0.002180416 |
| | X - 14302 | 0.12 | 0.000684491 | 0.000601044 | 0.14 | 0.001480759 | 0.001083098 |
| | X - 14314 | 0.19 | 7.14322E−05 | 8.29755E−05 | 0.21 | 0.000164475 | 0.000157507 |
| | X - 14320 | 0.31 | 1.7931E−07 | 5.9549E−07 | 0.29 | 3.939E−08 | 1.3214E−07 |
| | X - 14337 | 1.39 | 0.043736895 | 0.02054707 | 1.4 | 0.039935907 | 0.018138255 |
| | X - 14383 | 0.92 | 0.217392885 | 0.080908955 | 0.9 | 0.236143955 | 0.084299612 |
| | X - 14454 | 0.5 | 0.268044759 | 0.096693681 | 0.49 | 0.232079089 | 0.08306583 |
| | X - 14502 | 3.6 | 2.64312E−05 | 3.721E−05 | 3.59 | 2.66382E−05 | 3.35585E−05 |
| | X - 14658 | 0.01 | 1.0501E−07 | 3.8311E−07 | 0.01 | 2.018E−08 | 7.361E−08 |
| | X - 14697 | 0.19 | 1.6107E−07 | 5.4899E−07 | 0.18 | 1.19E−08 | 4.908E−08 |
| | X - 14838 | 1.82 | 3.48797E−05 | 4.56318E−05 | 1.78 | 4.61459E−05 | 5.32622E−05 |
| | X - 14904 | 0.6 | 0.041605025 | 0.019811045 | 0.58 | 0.034326803 | 0.015935871 |
| | X - 15136 | 0.76 | 0.413182986 | 0.137276827 | 0.73 | 0.276318055 | 0.095372564 |
| | X - 15461 | 1.75 | 0.708463724 | 0.205047788 | 1.85 | 0.564767888 | 0.164855884 |
| | X - 15486 | 0.38 | 0.649284678 | 0.192521066 | 0.37 | 0.607184537 | 0.174007477 |
| | X - 15497 | 8.97 | 1E−11 | 3E−10 | 17.23 | 1.00E−15 | 1.63E−13 |
| | X - 15503 | 1.71 | 0.006476538 | 0.004152619 | 1.65 | 0.011044058 | 0.006080709 |
| | X - 15523 | 0.73 | 0.44143589 | 0.143068195 | 0.7 | 0.373669549 | 0.120721322 |
| | X - 15646 | 3.43 | 5.81368E−06 | 1.01754E−05 | 3.39 | 1.07944E−05 | 1.56999E−05 |
| | X - 15666 | 1.92 | 0.652785439 | 0.19303091 | 1.99 | 0.572769074 | 0.166035731 |
| | X - 15853 | 0.35 | 0.010157724 | 0.006050981 | 0.34 | 0.007549641 | 0.004387126 |
| | X - 16071 | 0.69 | 0.41064308 | 0.136724208 | 0.7 | 0.530842246 | 0.159738709 |
| | X - 16083 | 1.27 | 0.923807476 | 0.248752186 | 1.24 | 0.980326415 | 0.254296682 |
| | X - 16343 | 1.09 | 0.589641212 | 0.179692277 | 1.05 | 0.738638805 | 0.201796771 |
| | X - 16580 | 0.59 | 0.269509939 | 0.096693681 | 0.57 | 0.329397934 | 0.108912552 |
| | X - 16654 | 3.02 | 1.71648E−05 | 2.51204E−05 | 2.92 | 3.93605E−05 | 4.64968E−05 |
| | X - 16946 | 0.56 | 0.106013996 | 0.043938345 | 0.55 | 0.11074038 | 0.043537928 |
| | X - 17009 | 0.31 | 0.916075626 | 0.24718414 | 1.52 | 0.202097338 | 0.07369758 |
| | X - 17299 | 1.6 | 0.001361829 | 0.001068979 | 1.67 | 0.000750123 | 0.000606895 |
| | X - 17327 | 0.94 | 0.456463015 | 0.147065246 | 0.91 | 0.501124368 | 0.151917971 |
| | X - 17328 | 2.51 | 0.007006012 | 0.004394215 | 2.64 | 0.004342511 | 0.00278029 |
| | X - 17674 | 3.49 | 4.48865E−05 | 5.75605E−05 | 3.83 | 2.50359E−05 | 3.21402E−05 |
| | X - 17807 - retired for | 2.9 | 0.000478021 | 0.000440658 | 2.91 | 0.000476175 | 0.000407532 |
| | asp-pro | | | | | | |
| | X - 17852 | 0.86 | 0.930384714 | 0.250003471 | 0.88 | 0.95304743 | 0.249633282 |
| | X - 17855 | 0.08 | 8.50277E−05 | 0.000096602 | 0.09 | 0.000182103 | 0.000171612 |
| | X - 17877 | 0.88 | 0.953890723 | 0.255524565 | 0.86 | 0.985197448 | 0.255134788 |
| | X - 17969 | 0.52 | 0.211223049 | 0.079067083 | 0.5 | 0.14779361 | 0.055669961 |
| | X - 17978 | 2.53 | 0.529784198 | 0.165540772 | 2.64 | 0.446938603 | 0.139259649 |
| | X - 17984 | 0.69 | 0.441052379 | 0.143068195 | 0.67 | 0.392199762 | 0.125393295 |
| | X - 18059 | 0.39 | 0.098508299 | 0.041156803 | 0.37 | 0.086066554 | 0.035155674 |
| | X - 18162 | 0.41 | 0.000638564 | 0.000570383 | 0.39 | 0.000240772 | 0.000217923 |
| | X - 18165 | 1.04 | 0.846049983 | 0.233643492 | 1.01 | 0.946162511 | 0.248508902 |
| | X - 18167 | 0.59 | 0.063079757 | 0.02788386 | 0.57 | 0.036903983 | 0.016975698 |
| | X - 18307 | 1.61 | 0.323652551 | 0.112988862 | 1.57 | 0.365490882 | 0.118816652 |
| | X - 18345 | 3.67 | 3.54368E−06 | 6.80057E−06 | 4.09 | 1.26157E−06 | 2.40769E−06 |
| | X - 18889 | 12.45 | 8.60E−12 | 2.8E−10 | 28.32 | 7.00E−15 | 4.35E−13 |
| | X - 19434 | 2.11 | 0.001500214 | 0.001163502 | 2.07 | 0.002166242 | 0.001497433 |
| | X - 19561 | 6.09 | 1.1E−11 | 3.1E−10 | 14.62 | 0.00E+00 | 9.60E−14 |
| | X - 19931 | 0.44 | 0.003856333 | 0.002656729 | 0.43 | 0.003539242 | 0.002313083 |
| | X - 20100 | 0.75 | 0.550527002 | 0.170378062 | 0.73 | 0.498839398 | 0.151590551 |
| | X - 20172 | 1.33 | 0.019298423 | 0.010480064 | 1.31 | 0.022838297 | 0.011356773 |
| | X - 20185 | 0.83 | 0.687390903 | 0.199841915 | 0.81 | 0.728579953 | 0.200353921 |
| | X - 20197 | 0.94 | 0.908638766 | 0.245689309 | 0.94 | 0.86587434 | 0.231038789 |
| | X - 20747 | 1.33 | 0.023898837 | 0.012557129 | 1.39 | 0.016796818 | 0.00871419 |
| | X - 21283 | 1.99 | 4.73777E−05 | 5.98661E−05 | 2.13 | 1.22081E−05 | 1.72571E−05 |
| | X - 21285 | 2.13 | 0.003592313 | 0.002535529 | 2.28 | 0.001830392 | 0.001304701 |
| | X - 21353 | 0.28 | 0.064062509 | 0.028126289 | 0.27 | 0.088090848 | 0.03586609 |
| | X - 21364 | 2.24 | 0.000687354 | 0.000601519 | 2.18 | 0.001238127 | 0.000924437 |
| | X - 21410 | 2.35 | 9.90686E−05 | 0.000109668 | 2.42 | 7.87353E−05 | 8.22042E−05 |
| | X - 21448 | 0.84 | 0.468467145 | 0.150185596 | 0.86 | 0.55638062 | 0.163546009 |
| | X - 21467 | 1.37 | 0.578054716 | 0.176785257 | 1.32 | 0.668178268 | 0.187222367 |
| | X - 21470 | 2.06 | 0.001274042 | 0.001014319 | 2.01 | 0.002123744 | 0.001476166 |
| | X - 21471 | 1.06 | 0.529511809 | 0.165540772 | 1.07 | 0.453109744 | 0.140753792 |
| | X - 21729 | 0.02 | 1.0623E−05 | 1.70916E−05 | 0.02 | 3.75223E−06 | 6.13071E−06 |
| | X - 21785 | 1.77 | 0.000722652 | 0.000626063 | 1.78 | 0.000650713 | 0.000538589 |
| | X - 21796 | 1.16 | 0.377970764 | 0.128319949 | 1.14 | 0.476040792 | 0.146531424 |
| | X - 22035 | 3.28 | 1.4387E−07 | 4.9691E−07 | 3.33 | 1.3615E−07 | 3.6062E−07 |
| | X - 22092 | 0.41 | 0.000505693 | 0.000461242 | 0.4 | 0.000304877 | 0.000270115 |
| | X - 22102 | 6.3 | 3.39188E−05 | 4.48276E−05 | 12.4 | 6.45046E−06 | 9.89666E−06 |
| | X - 22162 | 2.47 | 1.09533E−05 | 1.75142E−05 | 2.37 | 2.73497E−05 | 3.42371E−05 |
| | X - 23109 | 2.27 | 0.006246643 | 0.004037938 | 2.23 | 0.007050442 | 0.004127237 |
| | X - 23157 | 2.53 | 0.008516707 | 0.005215451 | 2.45 | 0.012454453 | 0.00676839 |
| | X - 23159 | 2.63 | 0.001004674 | 0.000836807 | 2.51 | 0.002130886 | 0.001477051 |
| | X - 23160 | 3.27 | 0.012840436 | 0.007375029 | 3.54 | 0.006927405 | 0.004082108 |
| | X - 23188 | 8.03 | 8.08E−09 | 4.549E−08 | 9.97 | 1.52E−09 | 8.5E−09 |
| | X - 23193 | 1.47 | 0.111932115 | 0.04587733 | 1.51 | 0.087869154 | 0.035833811 |
| | X - 23308 | 1.17 | 0.19165158 | 0.07289971 | 1.18 | 0.161558274 | 0.060223661 |
| | X - 23423 | 5.04 | 4.9E−09 | 3.252E−08 | 8.1 | 1.1E−11 | 1.6E−10 |
| | X - 23429 | 0.82 | 0.294053343 | 0.104200368 | 0.79 | 0.382024545 | 0.122823653 |
| | X - 23436 | 15.58 | 3.9E−10 | 4.59E−09 | 25.01 | 3.4E−10 | 2.35E−09 |
| | X - 23451 | 2.8 | 0.864804028 | 0.237304632 | 2.72 | 0.910095001 | 0.24079526 |
| | X - 23453 | 0.45 | 0.000848603 | 0.000720718 | 0.44 | 0.000523127 | 0.00044469 |
| | X - 23482 | 3.58 | 4.68474E−05 | 5.94862E−05 | 3.71 | 3.16785E−05 | 3.83215E−05 |
| | X - 23502 | 17.31 | 3.3E−10 | 4.28E−09 | 18.68 | 4.6E−10 | 3.02E−09 |
| | X - 23507 | 3.56 | 0.000144787 | 0.000152459 | 3.5 | 0.000175537 | 0.000166046 |
| | X - 23581 | 1.96 | 0.158534544 | 0.061939955 | 2.72 | 0.068271741 | 0.028441059 |
| | X - 23637 | 7.38 | 7E−11 | 1.32E−09 | 8.43 | 1E−11 | 1.6E−10 |
| | X - 23639 | 1.33 | 0.009765135 | 0.005882614 | 1.4 | 0.005721984 | 0.003494549 |
| | X - 23641 | 3.09 | 7.10143E−05 | 8.28617E−05 | 3.26 | 4.42498E−05 | 5.17863E−05 |
| | X - 23644 | 1.32 | 0.109720642 | 0.045042189 | 1.33 | 0.094013804 | 0.037970411 |
| | X - 23652 | 1.28 | 0.269161994 | 0.096693681 | 1.24 | 0.281249092 | 0.09659274 |
| | X - 23653 | 0.77 | 0.671576392 | 0.196790288 | 0.75 | 0.585417908 | 0.168923955 |
| | X - 23657 | 2.97 | 9.7662E−07 | 2.45895E−06 | 3.7 | 6.597E−08 | 0.0000002 |
| | X - 23662 | 13.59 | 0.055276245 | 0.02498654 | 13.23 | 0.060817628 | 0.026025188 |
| | X - 23680 | 2 | 0.00132217 | 0.00104737 | 2.05 | 0.00112499 | 0.000855189 |
| | X - 23710 | 6.91 | 6.37E−09 | 3.895E−08 | 20.75 | 4E−11 | 3.7E−10 |
| | X - 23728 | 0.05 | 0.029729605 | 0.015070537 | 0.05 | 0.028630871 | 0.013774461 |
| | X - 23729 | 0.86 | 0.787246297 | 0.222140861 | 0.83 | 0.693683791 | 0.192440281 |
| | X - 23734 | 1.87 | 0.361099389 | 0.123076083 | 1.78 | 0.298898641 | 0.100944195 |
| | X - 23736 | 1.84 | 0.000222576 | 0.000222607 | 1.84 | 0.000336517 | 0.000295031 |
| | X - 23737 | 2.47 | 0.000352091 | 0.000336547 | 3.21 | 3.42774E−05 | 0.000041267 |
| | X - 23738 | 1.87 | 0.047040261 | 0.021759164 | 2.21 | 0.016588491 | 0.008623891 |
| | X - 23739 | 3.84 | 4.71E−09 | 3.214E−08 | 4.17 | 1.77E−09 | 9.69E−09 |
| | X - 23747 | 0.62 | 0.526147035 | 0.164801866 | 0.6 | 0.537430244 | 0.160793256 |
| | X - 23756 | 1.86 | 0.019905907 | 0.010697824 | 1.99 | 0.010241993 | 0.005688896 |
| | X - 23767 | 0.32 | 0.033781283 | 0.016541732 | 0.31 | 0.029372818 | 0.01402531 |
| | X - 23776 | 1.19 | 0.429592808 | 0.141338711 | 1.21 | 0.371808773 | 0.12040384 |
| | X - 23782 | 0.47 | 0.668776262 | 0.19619171 | 0.46 | 0.730989059 | 0.200796956 |
| | X - 23787 | 3.55 | 2.70846E−06 | 5.5057E−06 | 4.49 | 2.2597E−07 | 5.7433E−07 |
| | X - 23906 | 3.27 | 5.56362E−05 | 6.83023E−05 | 3.71 | 1.56201E−05 | 2.1595E−05 |
| | X - 23908 | 10.81 | 1.02965E−06 | 2.49956E−06 | 12.45 | 2.6941E−07 | 6.4954E−07 |
| | X - 23925 | 4.04 | 3.97327E−06 | 7.40447E−06 | 4.84 | 1.28156E−06 | 2.42058E−06 |
| | X - 24077 | 1.54 | 0.016116086 | 0.009075326 | 1.56 | 0.0154611 | 0.008155747 |
| | X - 24216 | 0.27 | 0.001973523 | 0.001503571 | 0.25 | 0.001139775 | 0.000863819 |
| | X - 24220 | 0.86 | 0.161645225 | 0.062881917 | 1.26 | 0.055518601 | 0.02408531 |
| | X - 24229 | 0.98 | 0.686735168 | 0.199841915 | 0.93 | 0.637911408 | 0.181162527 |
| | X - 24231 | 3.74 | 2.60589E−06 | 5.35731E−06 | 3.57 | 7.85765E−06 | 1.17686E−05 |
| | X - 24238 | 1.65 | 0.019203746 | 0.010450559 | 2.23 | 0.00126186 | 0.000939369 |
| | X - 24243 | 2.32 | 0.000680313 | 0.000599407 | 2.51 | 0.000284019 | 0.00025523 |
| | X - 24246 | 10.35 | 0.108409629 | 0.044716594 | 10 | 0.121832787 | 0.04708959 |
| | X - 24329 | 1.74 | 0.006682431 | 0.004232254 | 1.67 | 0.012266831 | 0.006680856 |
| | X - 24348 | 1.47 | 0.171086885 | 0.066047245 | 1.43 | 0.215980019 | 0.078081124 |
| | X - 24352 | 2.48 | 0.008225541 | 0.005073126 | 4 | 0.00173413 | 0.001246681 |
| | X - 24356 | 2.48 | 2.95066E−05 | 0.000039891 | 2.64 | 1.88815E−05 | 2.52709E−05 |
| | X - 24359 | 1.22 | 0.359357988 | 0.122805722 | 1.18 | 0.476527875 | 0.146531424 |
| | X - 24400 | 5.1 | 1.524E−08 | 7.59E−08 | 6.02 | 4.3E−09 | 2.08E−08 |
| | X - 24408 | 2.56 | 0.019478207 | 0.010511586 | 3.09 | 0.007053199 | 0.004127237 |
| | X - 24413 | 0.06 | 0.043480075 | 0.020478027 | 0.05 | 0.041531619 | 0.018734551 |
| | X - 24425 | 3.53 | 4.35756E−06 | 7.94906E−06 | 3.95 | 1.14978E−06 | 2.24267E−06 |
| | X - 24431 | 4.64 | 2.87685E−05 | 3.92215E−05 | 4.64 | 2.99966E−05 | 3.66392E−05 |
| | X - 24435 | 0.99 | 0.789184654 | 0.222445501 | 0.96 | 0.635231389 | 0.180605263 |
| | X - 24452 | 2.1 | 8.31125E−05 | 0.000094842 | 2.24 | 2.66741E−05 | 3.35585E−05 |
| | X - 24455 | 1.34 | 0.444500578 | 0.14356827 | 1.36 | 0.398116848 | 0.126801733 |
| | X - 24456 | 2.17 | 0.010619004 | 0.006287452 | 2.29 | 0.006061402 | 0.003672135 |
| | X - 24464 | 0.52 | 0.169584017 | 0.065662786 | 0.51 | 0.166028171 | 0.061707057 |
| | X - 24474 | 2.9 | 1.2724E−07 | 4.5151E−07 | 3.05 | 5.087E−08 | 1.6545E−07 |
| | X - 24512 | 1.98 | 3.35542E−05 | 4.45731E−05 | 2.06 | 2.8678E−05 | 3.53721E−05 |
| | X - 24513 | 1.75 | 0.011423933 | 0.006679938 | 1.68 | 0.017231679 | 0.008921401 |
| | X - 24542 | 2.37 | 0.00017787 | 0.000183565 | 2.29 | 0.000384875 | 0.000335092 |
| | X - 24544 | 2.26 | 0.000164084 | 0.000170435 | 2.46 | 5.67665E−05 | 6.32012E−05 |
| | X - 24545 | 1.39 | 0.703663633 | 0.204114639 | 1.37 | 0.728004584 | 0.200353921 |
| | X - 24546 | 1.87 | 0.001240261 | 0.000997923 | 1.84 | 0.001854062 | 0.001314128 |
| | X - 24549 | 0.15 | 0.000293695 | 0.000284374 | 0.14 | 0.000190234 | 0.000178606 |
| | X - 24551 | 1.34 | 0.656474036 | 0.19345911 | 1.31 | 0.727142097 | 0.200353921 |
| | X - 24552 | 1.17 | 0.762087938 | 0.217162283 | 1.13 | 0.641681226 | 0.182017915 |
| | X - 24555 | 1.06 | 0.792245634 | 0.223065565 | 1.03 | 0.693585867 | 0.192440281 |
| | X - 24558 | 1.12 | 0.677213303 | 0.198217829 | 1.08 | 0.560817776 | 0.164367594 |
| | X - 24559 | 1.09 | 0.705605862 | 0.204449083 | 1.05 | 0.594136024 | 0.170904957 |
| | X - 24574 | 2.07 | 0.000941768 | 0.000792052 | 2.05 | 0.001194796 | 0.000900096 |
| | X - 24586 | 1 | | | 1 | | |
| | X - 24658 | 3.33 | 0.426438418 | 0.140717196 | 3.67 | 0.313807476 | 0.104860043 |
| | X - 24660 | 0.2 | 0.001276531 | 0.001014319 | 0.19 | 0.000362007 | 0.000316276 |
| | X - 24669 | 0.09 | 6.5844E−05 | 7.75272E−05 | 0.08 | 2.17073E−05 | 2.85413E−05 |
| | X - 24670 | 0.02 | 8.3054E−07 | 2.21794E−06 | 0.02 | 4.0326E−07 | 9.4214E−07 |
| | X - 24682 | 1.75 | 0.00775366 | 0.004816496 | 1.81 | 0.004921189 | 0.003064999 |
| | X - 24683 | 0.22 | 3.54324E−05 | 4.61219E−05 | 0.21 | 3.5457E−05 | 4.24839E−05 |
| | X - 24686 | 2.96 | 1.41E−09 | 1.22E−08 | 3.25 | 1.1E−10 | 8.9E−10 |
| | X - 24693 | 1.69 | 0.107659001 | 0.044548852 | 1.63 | 0.132213828 | 0.050635282 |
| | X - 24697 | 5.29 | 1.993E−08 | 9.561E−08 | 8.14 | 3E−10 | 2.16E−09 |
| | X - 24699 | 1.74 | 0.00014931 | 0.000156586 | 1.86 | 3.57387E−05 | 4.26184E−05 |
| | X - 24728 | 2.09 | 0.000918309 | 0.000774838 | 2.33 | 0.000548694 | 0.000463293 |
| | X - 24729 | 0.81 | 0.316819296 | 0.110966764 | 0.8 | 0.298433165 | 0.100928977 |
| | X - 24738 | 1.07 | 0.60650712 | 0.183751247 | 1.05 | 0.666555643 | 0.186975924 |
| | X - 24762 | 2.28 | 5.14933E−05 | 6.44378E−05 | 2.35 | 3.88692E−05 | 4.61329E−05 |
| | X - 24763 | 4.17 | 1.218E−08 | 6.309E−08 | 5.15 | 3.43E−09 | 1.729E−08 |
| | X - 24766 | 1.44 | 0.01722969 | 0.009577498 | 1.46 | 0.013648951 | 0.007354008 |
| | X - 24809 | 2.8 | 0.045915214 | 0.021314881 | 2.86 | 0.037836032 | 0.017341033 |
| | X - 24813 | 2.12 | 0.004878006 | 0.003231663 | 2.26 | 0.002365751 | 0.001621977 |
| | X - 24829 | 3.29 | 0.006250914 | 0.004037938 | 3.63 | 0.002888665 | 0.001928516 |
| | X - 24831 | 1.93 | 0.128168089 | 0.051553072 | 1.96 | 0.114946379 | 0.044920052 |
| | X - 24832 | 1.44 | 0.296573139 | 0.104949709 | 1.49 | 0.243344954 | 0.086403854 |
| | X - 24849 | 1.18 | 0.352075665 | 0.120795168 | 1.15 | 0.424174486 | 0.133389612 |
| | X - 24931 | 1.9 | 0.088830027 | 0.037673588 | 1.81 | 0.11707243 | 0.045599894 |
| | X - 24932 | 4.64 | 3.42449E−05 | 4.50288E−05 | 5.99 | 8.18365E−06 | 1.21843E−05 |
| | X - 24948 | 1.83 | 0.006571739 | 0.004192903 | 1.98 | 0.002821048 | 0.00190302 |
| | X - 24952 | 1.32 | 0.018360281 | 0.010076224 | 1.33 | 0.015325398 | 0.008101148 |

Table 1 contains a complete list of all metabolites analyzed and represented as a ratio of intervention/control. These data represent relative abundance between the 2 groups. The significant increase or decrease is represented by a p-value less than 0.05. It is also denoted in bold in the table. An intervention/control ratio number greater than 1 indicates that the metabolite is higher in the intervention compared to the control. A number less than 1 indicates that the metabolite is lower in the intervention compared to the control.

### Example 3. Elevating Levels of Conjugated Lactate Metabolites, Tryptophan Precursors and Serotonin in the Gut of a Human Infant

Serotonin is an important neurotransmitter in the body that has important roles in the brain-gut axis and may contribute to improved sleep, cognition, gut motility, and satiety. Tryptophan is a precursor to serotonin. Indolelactate, a bacterial metabolite is a metabolite that can serve as a valuable precursor for tryptophan and serotonin metabolism for the host. Indolelactate demonstrates the symbiotic relationship between bacteria and host. In a broader application, lactate is an important metabolite for brain function and other conjugated lactate metabolites may serve to increase available lactate to the brain. The compositions and methods of this invention provide a continuous source of conjugated lactate derivatives such as, but not limited to, indolelactate, phenyllactate, and 3-(4-hydroxyphenyl)lactate when mammalian milk oligosaccharides are provided as all or part of the fiber component of the diet.

An untargeted metabolomics analysis was completed on fecal samples collected in Example 1 from 20 infants at day 28 who were receiving the standard of care. The same analysis was completed on samples collected in Example 1 from 20 newborn infants receiving a composition of B. infantis and human milk oligosaccharides. The relative abundance of serotonin and indolelactate metabolites were analyzed, and the results are reported in Table 2 below.

TABLE 2
Relative abundances of serotonin and conjugated lactate metabolites.
The bold values are significant. The p-value is noted in
column 3. A value above 1 means it is increased in Intervention
compared to control while a number below 1 means it is decreased
in intervention compared to control.

| | Intervention/ | |
|---|---|---|
| Metabolite | Control | P-value |
| serotonin | 3.6 | 0.00000028 |
| indolelactate | 10.0 | 0.00000002 |
| phenyllactate | 4.2 | 0.00011 |
| 3-(4-hydroxyphenyl)lactate | 16.7 | 0.0000000000012 |

In a set of fecal samples from day 40-50, serotonin in control infants was measured to have an average of 0.1 ng/mg feces compared to infants receiving B. infantis and HMO who had an average of 0.45 ng/mg feces using an ELISA kit.

### Example 4. Elevating Levels of Hippurate in the Gut of a Human Infant

Hippurate is a metabolite that is important in the detoxification of benzoic acid and other polyphenols. The detoxification of the benzoic acid requires a source of glycine. Glycine is a conditionally essential amino acid. In cases where benzoic acid detoxification is required, it can deplete glycine and limits its availability for other important metabolic functions.

An untargeted metabolomics analysis was completed on fecal samples collected in Example 1 from 20 infants at day 28 who were receiving the standard of care. The same analysis was completed on samples collected in Example 1 from 20 newborn infants receiving a composition of B. infantis and human milk oligosaccharides. The relative abundance of hippurate-related metabolites were analyzed, and the results are reported in Table 3 below.

TABLE 3
Significant changes in the Benzoate Metabolic Pathway.
The bold values are significant. The p-value is noted
in column 3. A value above 1 means it is increased in
Intervention compared to control while a number below
1 means it is decreased in intervention compared to control.

| | Intervention/ | |
|---|---|---|
| Metabolite | Control | P value |
| hippurate | 2.2 | 0.0027 |
| 3-hydroxyhippurate | 3.3 | 0.0000649 |
| 4-hydroxyhippurate | 2.3 | 0.0000267 |
| benzoate | 0.3 | 0.00067 |
| 4-hydroxybenzoate | 0.5 | 0.00069 |

Infants treated with a composition comprising B. infantis and human milk oligosaccharides had 2- to 3-fold increases in hippurate, 3-hydroxyhippurate, 4 hydroxyhippurate with a significant reduction in benzoate and 4-hydroxybenzoate. The methods of this invention provide a means of delivering more amino acids (see Table 1) to the intestine, and/or an organism capable of conjugating benzoic acid and glycine to form hippurate. It may also displace hippurate-degrading microbes such as, but not limited to, Group B streptococcus and Camplyobacter jejuni.

### Example 5. Elevate Levels of Metabolites Gamma-Glutamylcysteine and Creatinine in the Gut of a Human Infant

Creatinine and gamma-glutamyl cysteine and other gamma-glutamyl amino acids are important for preventing and/or recovering from oxidative stress. Gamma-glutamyl cysteine is an important precursor for glutathione (GSH). It is an integral part of preventing oxidative stress in a mammal. Creatinine is an important metabolite to reduce the effects of oxidative stress and can be instrumental in preventing oxidation mediated mitochondrial damage in premature and high risk deliveries. Oxidative stress is a condition that occurs during the birthing process. In term infants, GSH is generally sufficient, but it may not be in preterm infants, and it may also be low in people with autism. Autism is a spectrum of disorders and is best treated early in life to minimize the severity. Diagnosis generally occurs after some critical windows have closed. Monitoring levels and recovery from oxidative stress during pregnancy and at birth may be an overall indicator of health and can be a tool to minimize long-term sub-clinical effects of early oxidative stress by administering the compositions in this invention.

An untargeted metabolomics analysis was completed on fecal samples collected in Example 1 from 20 infants at day 28 who were receiving the standard of care. The same analysis was completed on samples collected in Example 1 from 20 newborn infants receiving a composition of B. infantis and human milk oligosaccharides. The relative abundance of glutamyl-dipeptide metabolites were analyzed, and the results are reported in Table 4 below.

TABLE 4
significant changes in the gamma-glutamyl amino acids.
The bold values are significant. The p-value is noted
in column 3. A value above 1 means it is increased in
Intervention compared to control while a number below
1 means it is decreased in intervention compared to control.

| | Active/ | |
|---|---|---|
| Metabolites | Control | P-value |
| gamma-glutamylalanine | 5.9 | 7.532E−08 |
| gamma-glutamylcysteine | 44.3 | 1.17E−12 |
| gamma-glutamylglutamate | 3.1 | 0.004279002 |
| gamma-glutamylglutamine | 1.6 | 0.001325298 |
| gamma-glutamylhistidine | 10.5 | 6.88631E−05 |
| gamma-glutamylisoleucine* | 8.1 | 8.144E−07 |
| gamma-glutamylleucine | 1.6 | 0.025584145 |
| gamma-glutamyl-alpha-lysine | 3.3 | 0.052765195 |
| gamma-glutamyl-epsilon-lysine | 2.4 | 1.66338E−05 |
| gamma-glutamylmethionine | 19.3 | 8.75E−09 |
| gamma-glutamylphenylalanine | 4.2 | 5.67663E−05 |
| gamma-glutamyltyrosine | 2.8 | 0.000714584 |
| gamma-glutamylvaline | 3.3 | 1.11065E−05 |
| creatinine | 1.4 | 0.021567567 |

Creatinine and/or gamma-glutamyl cysteine can be used as metabolic indicators for monitoring levels pre and post-intervention and/or determining the need for an intervention to improve the health of said infant.

### Example 6. Elevation of Pipecolate in the Infant Gut

Colorectal cancer (CRC) is an important public health problem, accounting worldwide for over 694,000 deaths in 2012. In the U.S., CRC was the fourth most common cancer and the second leading cause of cancer-related deaths in 2011. Accumulating evidence suggests that there is a protective role of dry bean intake against colorectal neoplasia occurrence and progression. In a controlled human feeding study (LIFE), a 4-wk high dry bean diet (250 g/d) favorably changed serum markers of inflammation, insulin resistance, and hypercholesterolemia, which are associated positively with CRC. In Perera et al., 2015 [Perera T et al. Mol Nutr Food Res. 2015 April; 59(4):795-806] a controlled human feeding study and a controlled mouse feeding study, serum was identified as a marker of dry bean consumption. In a multi-year intervention study that promoted dry bean consumption in the intervention arm only serum pipecolic acid (PA) separated individuals based on dry bean consumption. PA is a cyclic, non-protein imino acid, which is the most abundant non-protein nitrogen fraction in dry beans whereas most commonly consumed foods have negligible PA contents. Dietary PA may confer chemopreventive benefits for a human host because PA is a precursor of microbial compounds with anti-inflammatory, antitumor, and antibiotic properties. U.S. Patent Application Publication No. 2015/0211035 recites a method of producing PA requiring a recombinant microorganism that has had added genes involved in the biosynthetic pathway of pipecolic acid and a DNA encoding region for a protein that has L-pipecolic acid-cis-5-hydroxylase, and culturing that recombinant organism in a medium where PA can be recovered from that medium. The application further describes isolating the appropriate genes from Flavobacterium lutescens and expressing them in E. coli. Preterm infants had a higher excretion of PA than term neonates in urine. PA excretion of infants decreases with age after birth. PA level in serum and urine remains a valuable tool for the confirmation of the clinical diagnosis of Zellweger syndrome when gestational age and age after birth are taken into consideration. No PA was detected in serum or urine of four children suffering from hyperthyroidism (Govaerts et al. Inherit Metab Dis. (1985) 8(2):87-91). Pipecolic acid can be a precursor for the neurotransmitter piperdine.

An untargeted metabolomics analysis was completed on fecal samples collected from Example 1 from 20 infants at day 28 who were receiving the standard of care. The same analysis was completed on samples collected from Example 1 from 20 newborn infants receiving a composition of B. infantis and human milk oligosaccharides. The relative abundance of pipecolic acid metabolites were analyzed.

The fecal levels of pipecolate were significantly elevated in infants receiving a composition of B. infantis and human milk oligosaccharides. Thus, this invention provides an alternative method for introducing additional pipecolic acid into patients via the colon.

### Example 7. Alteration in Bile Acid Metabolites

Bile acids are important for lipid absorption and an important feature is their ability to be resorbed and recirculated (i.e recycled rather than requiring denovo synthesis). Cholate and chenodeoxycholate are the primary, unconjugated bile acids in humans. These are conjugated with glycine and taurine to form conjugated bile acids that can be reabsorbed. Secondary bile acids are further modified by the microbiome and can decrease the resorption and recycling of bile acids.

An untargeted metabolomics analysis was completed on fecal samples collected in Example 1 from 20 infants at day 28 who were receiving the standard of care. The same analysis was completed on samples collected in Example 1 from 20 newborn infants receiving a composition of B. infantis and human milk oligosaccharides. The relative abundance of bile acid metabolites were analyzed, and the results are reported in Table 5 below.

TABLE 5
metabolites changed in response to intervention for primary
and secondary bile acids compared to the standard group.

| | Intervention:Control | |
|---|---|---|
| Metabolite | Ratio | P-value |
| cholate | 3.2 | 0.002 |
| chenodeoxycholate | 4.9 | 0.003 |
| glycochenodeoxycholate | 0.01 | 0.0000013 |
| cholate sulfate | 5.6 | 0.000003 |
| glycochenodeoxycholate sulfate | 0.02 | 4.37814E−06 |
| glycocholate glucuronide (1) | 0.3 | 0.000488927 |
| glycocholate sulfate | 0.09 | 1.09357E−05 |
| glycolithocholate sulfate* | 0.03 | 4.3209E−07 |
| taurolithocholate 3-sulfate | 0.05 | 5.5168E−07 |
| tauroursodeoxycholate | 0.07 | 2.11966E−05 |
| 7-ketolithocholate | 0.31 | 0.044 |
| glycohyocholate | 0.02 | 0.0000025 |
| glycocholenate sulfate* | 0.06 | 0.000000063 |
| taurocholenate sulfate | 0.1 | 0.00006 |

The bold values are significant. The p-value is noted in column 3. A value above 1 means it is increased in Intervention compared to control while a number below 1 means it is decreased in intervention compared to control.

More quantitative information on the concentration and levels of the different primary and secondary metabolites measured in the fecal samples from this study can be obtained by more detailed analysis on a bile acid panel. Samples are extracted, spiked with a solution of labelled internal standards, evaporated to dryness, and reconstituted and injected onto an Agilent 1290/Sciex QTrap 6500 LC-MS/MS system equipped with a C18 reverse phase HPLC column with acquisition in negative ion mode. The peak area of each bile acid parent (pseudo-MRM mode) or product ion is measured against the peak area of the respective internal standard parent (pseudo-MRM mode) or product ion. In addition, host markers of endocrine and metabolic functions may also be examined.

Using spectrophotometer based assays for individual metabolites, overall bile acid were higher in the treated group (85 mM/mg feces) compared to the control group (12 mM/mg feces). Intervention resulted in a bile acid profile that had increased fecal cholate (190 vs 134 mm/mg feces;) and chenodeoxycholate (1433 vs 896 mM/mg feces). Overall branch chain amino acids (BCAA) were higher on average (435 μM/mg feces after intervention compared to 158 μM/mg feces in control). There was also a significant decrease in secondary bile acids: glycolithocholate sulfate, tauroursodeoxycholate, 7-ketolithocholate, glycohyocholate, glycocholenate sulfate, or taurocholenate sulfate. Thus, this invention provides a method for delivering improving lipid degradation and bile acid recycling into individuals, as well as reduction in bile acid malabsorption conditions or syndrome, plus improved water retention and stool consistency.

### Example 8 Alterations in Long Chain Fatty Acids

Long chain fatty acids are important for gut maturation that may include immune development and contribute to disease risk reduction. Increased abundance of long chain fatty acids following treatment with human milk and B. infantis are described in Table 1 and Table 6.

An untargeted metabolomics analysis was completed on fecal samples collected in Example 1 from 20 infants at day 28 who were receiving the standard of care. The same analysis was completed on samples collected in Example 1 from 20 newborn infants receiving a composition of B. infantis and human milk oligosaccharides. The relative abundance of fatty acid metabolites were analyzed, and the results are reported in Table 6 below.

TABLE 6
Long chain fatty acid elevated following treatment with HMO and B. infantis

| | | | | | | | |
|---|---|---|---|---|---|---|---|
| Long Chain | myristate (14:0) | 1.96 | 0.013220591 | 0.007576575 | 2.12 | 0.00747399 | 0.004353219 |
| Fatty Acid | pentadecanoate (15:0) | 3.1 | 1.47497E−05 | 2.22135E−05 | 3.31 | 6.69585E−06 | 1.02109E−05 |
| | palmitate (16:0) | 2.28 | 5.99834E−06 | 1.04281E−05 | 2.42 | 2.29022E−06 | 3.94698E−06 |
| | margarate (17:0) | 4.42 | 2.16588E−06 | 4.67535E−06 | 5 | 7.3456E−07 | 1.56634E−06 |
| | stearate (18:0) | 3.02 | 9.5102E−07 | 2.43909E−06 | 3.32 | 2.4349E−07 | 6.1268E−07 |
| | nonadecanoate (19:0) | 5.4 | 2.1146E−07 | 6.7626E−07 | 5.99 | 8.071E−08 | 2.3079E−07 |
| | 10-nonadecenoate | 1.51 | 0.047594393 | 0.021976242 | 1.61 | 0.03127373 | 0.014763666 |
| | (19:1n9) | | | | | | |
| | trans-nonadecenoate | 1.59 | 0.001107784 | 0.000908089 | 2.6 | 5.00588E−05 | 5.68612E−05 |
| | (tr 19:1)* | | | | | | |
| | arachidate (20:0) | 4.69 | 2.48696E−06 | 5.1537E−06 | 5.54 | 6.3743E−07 | 1.38266E−06 |
| | eicosenoate (20:1) | 1.57 | 0.01205826 | 0.007003422 | 1.69 | 0.006990048 | 0.004109397 |
| | behenate (22:0)* | 3.82 | 7.81399E−06 | 1.31435E−05 | 4.37 | 2.40999E−06 | 4.12515E−06 |
| | erucate (22:1n9) | 1.58 | 0.012137278 | 0.007017845 | 1.71 | 0.006775234 | 0.004011226 |
| | nervonate (24:1n9)* | 2.17 | 0.00191334 | 0.001462018 | 2.4 | 0.000915545 | 0.000722156 |
| | docosadienoate | 1.65 | 0.019629795 | 0.010571368 | 1.75 | 0.012819211 | 0.006936655 |
| | (22:2n6) | | | | | | |
| | dihomo-linoleate | 1.35 | 0.043791896 | 0.02054707 | 1.45 | 0.025922489 | 0.012665175 |
| | (20:2n6) | | | | | | |
| Fatty Acid | PAHSA (16:0/OH-18:0) | 9.91 | 1.52E−09 | 1.271E−08 | 21.04 | 4E−11 | 3.7E−10 |
| Hydroxyl | LAHSA (18:2/OH- | 4.48 | 5.7188E−07 | 1.59288E−06 | 6.53 | 6.378E−08 | 1.957E−07 |
| Fatty Acid | 18:0)* | | | | | | |
| and | OAHSA (18:1/OH- | 5.11 | 1.7406E−07 | 5.8557E−07 | 6.69 | 3.432E−08 | 1.1831E−07 |
| Branched | 18:0)* | | | | | | |
| fatty acids | 15-methylpalmitate | 2.23 | 0.000186517 | 0.000189469 | 2.36 | 0.000109084 | 0.000108372 |
| | 17-methylstearate | 4.44 | 2.98152E−06 | 5.98699E−06 | 4.94 | 1.14763E−06 | 2.24267E−06 |

1-8. (canceled)

9. A method of changing the amount of one or more metabolites in the intestine of a mammal, comprising:
a) administering a bacterial composition comprising bacteria capable of colonization of the intestine; and
b) administering OS;
wherein the bacteria and the OS are administered in respective amounts sufficient to change the amount of the one or more metabolites in the intestine of said mammal.

10. (canceled)

11. The method of claim 9, wherein the one or more metabolites is selected from the compounds listed in column 2 of Table 1.

12. The method of claim 11, wherein the one or more metabolites comprises a metabolite selected from the group consisting of γ-glutamyl-containing di- or tri-peptides, γ-glutamyl-cysteine, pipecolic acid, hippurate, 3-hydroxyhippurate, 4-hydroxyhippurate, benzoic acid, cholate, cholate sulfate, chenodeoxycholate, choline, creatine, palmitic acid-9-hydroxy-stearic acid (PAHSA 16:0/OH-18:0), and Oleic Acid-Hydroxy Stearic Acid (OAHSA 18:1/OH-18:0), nonadeconate, arachinate, eiocosenoate, stearate, 15-methylplamitate, 17-methylpalmitate, behenate, margarate, palmitate, myristate, citrate, succinate, serotonin, tryptophan, lactate conjugates, 3-4 hydroxyphenyl lactate, indole lactate, and phenyllactate.

13. The method of claim 12, wherein the concentration of γ-glutamyl-containing di- or tri-peptides is at least 10-fold greater than the level in the colon of a mammal which is not colonized by the bacteria, or wherein the concentration of γ-glutamyl-cysteine is at least 20-fold greater than the γ-glutamyl-cysteine abundance in the colon of a mammal which is not colonized by the administered bacteria.

14. (canceled)

15. The method of claim 12 where creatine is greater than the creatine abundance in the colon of a mammal which is not colonized by the administered bacteria.

16. The method of claim 12, wherein the concentration of pipecolic acid or salt thereof is at least 10-fold greater than the pipecolic acid level in the colon of a mammal which is not colonized by the bacteria.

17. The method of claim 12, wherein hippurate level is determined, said level of hippurate indicating detoxification of the intestine of said mammal,
or
further wherein supplemental glycine is added to the diet of an individual in need of benzoic acid or polyphenol detoxification,
or
wherein the amount of bacterial hippurate degraders are reduced in the microbiome.

18-21. (canceled)

22. The method of claim 12, wherein the concentration of cholate, chenodeoxycholate, cholate sulphate is greater than cholate, chenodeoxycholate, cholate sulphate abundance in the colon of a mammal which is not colonized by the administered bacteria.

23. The method of claim 12, wherein long chain fatty acid metabolites are increased compared to levels in a mammal which is not colonized by the bacteria, said fatty acid metabolites optionally including one or more of palmitic acid-9-hydroxy-stearic acid (PAHSA 16:0/OH-18:0), Oleic Acid-Hydroxy Stearic Acid (OAHSA 18:1/OH-18:0), nonadeconate, arachinate, eiocosenoate, stearate, 15-methylplamitate, 17-methylpalmitate, behenate, margarate, palmitate, myristate, or a combination thereof.

24. The method of claim 12, wherein the one or more metabolites comprise a TCA cycle metabolite, said TCA cycle metabolite optionally comprising citrate or succinate.

25. The method of claim 12, wherein tryptophan metabolites are increased compared to levels in a mammal which is not colonized by the bacteria, said tryptophan metabolites optionally including one or more of serotonin, tryptophan, lactate conjugates, 3-4 hydroxyphenyl lactate, indole lactate, and/or phenyllactate.

26-27. (canceled)

28. The method of claim 9 wherein said method reduces the risk of said mammal developing a metabolic disorder, said metabolic disorder being optionally selected from metabolic disorders consisting of Obesity and Type 2 Diabetes and combinations thereof, said risk being reduced as compared to a dysmetabolic mammal.

29-30. (canceled)

31. The method of claim 9, wherein said method reduces the risk of said mammal developing an autoimmune disease, said autoimmune disease optionally selected from the following auto-immune diseases: Juvenile Diabetes (Type I), asthma, atopy, Celiac's Disease, food allergies and combinations thereof, said risk being reduced as compared to a dysmetabolic mammal.

32-36. (canceled)

37. The method of claim 9 wherein said method reduces the risk of said mammal developing a cognitive disorder, said cognitive disorder being optionally selected from the following cognitive disorders: autism, depression, or learning disability, and combinations thereof, said risk being reduced as compared to a dysmetabolic mammal.

38-41. (canceled)

42. The method of claim 9, wherein the OS comprises mammalian milk oligosaccharide (MMO), and wherein the MMO comprises N-acetyl lactosamine, lacto N-tetrose, lacto-N-biose, N-acetyllactosamine, lacto-N-triose, lacto-N-neotriose, lacto-N-neotetrose, fucosyllactose, lacto-N-fucopentose, lactodifucotetrose, sialyllactose, di sialyllactone-N-tetrose, 2′-fucosyllactose, 3′-sialyllactosamine, 3′-fucosyllactose, 3′-sialyl-3-fucosyllactose, 3′-sialyllactose, 6′-sialyllactosamine, 6′-sialyllactose, difucosyllactose, lacto-N-fucosylpentose I, lacto-N-fucosylpentose II, lacto-N-fucosylpentose III, lacto-N-fucosylpentose V, sialyllacto-N-tetraose, their derivatives, or combinations thereof.

43. The method of claim 9, wherein the OS comprises MMO, and wherein the MMO consists of carbohydrate polymers found in mammalian milk which are not metabolized by any combination of digestive enzymes expressed by mammalian genes, or wherein the OS comprises a Type II oligosaccharide core where representative species include LNnT or LNFPIII and/or a Type I oligosaccharide core where representative species include LNT or LNFPI.

44-67. (canceled)

68. The method of claim 9, wherein the bacterial composition comprises bacteria of a genus selected from the group consisting of Bifidobacteria, Lactobacillus, and Pediococcus which may be of a species selected from B. adolescentis, B. animalis, B. animalis subsp. animalis, B. animalis subsp. lactis, B. bifidum, B. breve, B. catenulatum, B. longum, B. longum subsp. infantis, B. longum subsp. longum, B. pseudocatanulatum, B. pseudolongum, L. acidophilus, L. antri, L. brevis, L. casei, L. coleohominis, L. crispatus, L. curvatus, L. equi, L. fermentum, L. gasseri, L. johnsonii, L. mucosae, L. pentosus, L. plantarum, L. reuteri, L. rhamnosus, L. sakei, L. salivarius, P. acidilactici, P. argentinicus, P. claussenii, P. pentosaceus, P. stilesii L. paracasei, L. kisonensis, L. paralimentarius, L. perolens, L. apis, L. ghanensis, L. dextrinicus, L. shenzenensis, L. harbinensis, P. parvulus, or P. lolii.

69-78. (canceled)

79. The method of claim 9, wherein the mammal is a human or non-human mammal.

80-81. (canceled)

82. The method of claim 9, wherein the infant is a pre-term infant or a term infant.

83-87. (canceled)

88. A method of reducing the risk of Juvenile Diabetes (Type I), comprising:
a) administering a bacterial composition comprising at least one species capable of consuming MMO by the internalization of that MMO within the bacterial cell, and;
b) administering OS at a dose sufficient to maintain colonization of the colon of the mammal by bacteria in the bacterial composition of step (a), wherein the risk is reduced as compared to a dysbiotic infant;
wherein the bacteria and the OS are administered in respective amounts sufficient to increase the amount of the one or more metabolites in the intestine of said mammal;
wherein the one or more metabolites is selected from the compounds listed in column 2 of Table 1, or the one or more metabolites comprises a metabolite selected from the group consisting of γ-glutamyl-containing di- or tri-peptides, γ-glutamyl-cysteine, pipecolic acid, hippurate, 3-hydroxyhippurate, 4-hydroxyhippurate, benzoic acid, cholate, cholate sulfate, chenodeoxycholate, choline, creatine, palmitic acid-9-hydroxy-stearic acid (PAHSA 16:0/OH-18:0), and Oleic Acid-Hydroxy Stearic Acid (OAHSA 18:1/OH-18:0), nonadeconate, arachinate, eiocosenoate, stearate, 15-methylplamitate, 17-methylpalmitate, behenate, margarate, palmitate, myristate, citrate, succinate, serotonin, tryptophan, lactate conjugates, 3-4 hydroxyphenyl lactate, indole lactate, and phenyllactate.

89-96. (canceled)

97. The method according to claim 9, further comprising:
c) determining the amount and/or concentration, of one or more metabolites in a fecal sample obtained from the mammal;
d) identifying a dysmetabolic state in the mammal based on the amount and/or concentration of the one or more metabolites in the sample;
e) optionally wherein the bacterial composition and/or the OS is to be administered or re-administered in response to the identified dysmetabolic state;
f) optionally wherein the amount, periodicity, and/or duration of the administration of the bacterial composition and/or the OS is adjusted in response to an identified dysmetabolic state; and
g) optionally wherein the amount and/or type of the bacterial composition and/or the OS to be administered in step (a) is different than the amount and/or type of the bacterial composition and/or the OS to be administered in step (f).
